(19) United States
(12) Patent Application Publication
(10) Pub. No.: US 2014/0330000 A1
(43) Pub. Date: Nov. 6, 2014

(54) Molecular Cartesian-Coordinate Platforms

(71) Applicants: Michael D. Hopkins, Chicago, IL (US); Cameron P. Iverson, Westminster, CA (US); Wing-Yeung (Wayne) Lau, Chicago, IL (US)

(72) Inventors: Michael D. Hopkins, Chicago, IL (US); Cameron P. Iverson, Westminster, CA (US); Wing-Yeung (Wayne) Lau, Chicago, IL (US)

(21) Appl. No.: 14/008,013

(22) PCT Filed: Mar. 30, 2012
(86) PCT No.: PCT/US12/31346

§ 371 Date: Oct. 16, 2013

Related U.S. Application Data
(60) Provisional application No. 61/516,235, filed on Mar. 31, 2011.
(60) Provisional application No. 61/685,948, filed on Mar. 27, 2012.

(51) Int. Cl. C07D 487/22

(52) U.S. Cl. CPC C07D 487/22
USPC 534/701; 540/145; 540/140

(57) ABSTRACT

A compound of formula MOL-M-L, wherein M is a 5 or 6-coordinate metal, L is a bidentate ligand, and MOL is a compound of formula (II): (II) wherein X is C and R\ R2, R3, R4, R5, R6, R7, R8, R9, R10, R11 and R12 are as defined herein. In another embodiment, a system comprises a substrate and a compound of the present disclosure immobilized on the substrate. In still another embodiment, a method comprises introducing a reagent to a substrate presenting the compound of the present disclosure.

This application claims priority to U.S. Provisional Patent Application Ser. No. 61/516,235 to Hopkins et al., filed on Mar. 31, 2011, and entitled “Assembly Of Square-Pyramidal Metalloporphyrins On HOPG”; and U.S. Provisional patent application Ser. No. ______ to Hopkins et al., filed on Mar. 27, 2012 (attorney docket number 7814-342), and entitled “Assembly Of Square-Pyramidal Metalloporphyrins On HOPG”, the entireties of both of which are incorporated herein by reference.

This invention was made with government support under grant number DMR-0820054 awarded by NSF MRSEC Program. The government has certain rights in the invention.

### BACKGROUND

One of the basic tenets of molecular nanotechnology is that organized assemblies of nanometer-sized molecules or clusters may exhibit unique collective properties or functions and could enable the limits of device density to be approached. Whitesides and co-workers (Parviz, B. A.; Ryan, D.; Whitesides, G. M., IEEE Trans. Adv. Packaging 2003, 26 (3), 233-241) proposed that in order to meet the size needs for nanofabrication, there are five key elements that should be considered. “The technology must: i) be able to produce components with nanometer (or better) precision; ii) be able to assemble systems from these components; iii) be parallel in nature—producing many circuits and systems simultaneously; iv) be able to structure in three dimensions; v) be cost-effective.” While criterion (i) can be met intrinsically with molecular synthesis, the other criteria often pose steep challenges. One way to organize molecular components on the nanoscale is to guide them using lithographic patterning of surfaces. For example, dip-pen nanolithography can deliver molecules to substrates with sub-50 nm resolution, and electron beam lithography has achieved sub-20 nm feature size. These length scales are still much larger than those of small molecule functional components. A bottom-up approach to organizing nanoscale structures is to design molecular functional components that self-assemble into prescribed patterns. In particular, self-assembly of molecular overlayers on solid substrates offers a variety of applications in molecular electronics, information storage, catalysis, and chemical sensing. The self-assembly approach has produced examples that meet several of the criteria set aside by Whitesides

In considering design motifs for self-assembled nanoscale molecular systems, the idea of organizing these on surfaces has considerable appeal. This is both because a planar surface provides a two-dimensional (2D) constraint that is useful in limiting uncontrolled assembly in the third dimension (e.g., the conversion of a network to a crystal), and because an electroactive surface provides means to address the assembly and interface it to the micron world. Self-assembled molecular layers on surfaces can be roughly divided into two classes: close-packed monolayers of long chain molecules whose long axis is perpendicular to the surface, such as the archetypal self-assembled monolayers (SAM) of alkanethiols on gold; and open networks of planar molecules deposited coplanar to the surface, such as those formed by complimentary hydrogen bonding (H-bond) donor/acceptor pairs. If one were to use these networks to position molecular components at specific Cartesian x, y, and z coordinates relative to the surface, the alkanethiols could provide control over the z coordinate and the H-bonded network can provide control over the x and y coordinates, but neither readily allows full control over all three coordinates. Such control can be achieved by a new type of monolayer that combined the properties of the two.

### BRIEF SUMMARY

In one embodiment, the present disclosure provides a compound of formula (I)

MOL-M-L (I)

wherein M is a 5 or 6-coordinate metal, L is a bidentate ligand, and MOL is a compound of formula (II)

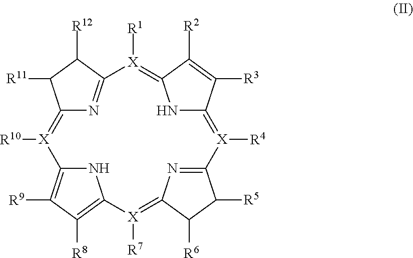

wherein X is C or N; R¹, R², R³, R⁴, R⁵, R⁶, R⁷, R⁸, R⁹, R¹⁰, R¹¹ and R¹² are each independently selected from the group consisting of hydrogen, halogen, substituted or unsubstituted C₁₋₈ alkyl, substituted or unsubstituted C₂₋₈ alkenyl, substituted or unsubstituted C₂₋₈ alkynyl, —N₃, —OCN, —SCN, —NO₃, —OTeF₅, trazolate, tetrazolate, —CN, —OR¹³, —OOR¹³, —CCR¹³, —OC(O)R¹³, —CO₂R¹³, —C(O)R¹³, —C(O)NR¹³R¹⁴, —OC(O)NR¹³R¹⁴, —NR¹⁴C(O)R¹³, —NR¹⁵C(O)NR¹³R¹⁴, —NR¹³R¹⁴, —NR¹⁴CO₂R¹³, —SR¹³, —S(O)R¹³, —S(O)₂R¹³, —S(O)₂NR¹³R¹⁴, —NR¹⁴S(O)₂R¹³, substituted or unsubstituted C₆₋₁₀ aryl, substituted or unsubstituted 5- to 10-membered heteroaryl, substituted or unsubstituted 3- to 10-membered cyclyl, and substituted or unsubstituted 3- to 10-membered heterocyclyl, or one or more pairs of R²/R³, R⁵/R⁶, R⁸/R⁹ and R¹¹/R¹² are each independently, together with the atom(s) to which they are attached, form an substituted or unsubstituted 3- to 10-membered ring,

wherein each occurrence of R¹³, R¹⁴ and R¹⁵ is independently selected from the group consisting of hydrogen, C₁₋₈ alkyl, C₂₋₈ alkenyl, C₂₋₈ alkynyl, aryl, or heteroaryl; or R¹³ and R¹⁴, together with the atom(s) to which they are attached, form an substituted or unsubstituted 3- to 10-membered ring.

In another embodiment, a system comprises a substrate and a first compound of the present disclosure immobilized on the substrate.

In yet another embodiment, a method comprises contacting a solution comprising a first compound of the present disclsoure with a substrate such that some of the first compound is immobilized to the substrate.

In still another embodiment, a method comprises introducing a reagent to a substrate presenting the compound of the present disclsoure.

### BRIEF DESCRIPTION OF THE DRAWINGS

FIG. 1. Depicts two functional modules, A and B, fixed at positions relative to each other and a planar surface by means of planar surface-supported molecules (M, M′ porphyrin or phthalocyanine) with rigid perpendicular ligands (L, L′). Groups R₁ and R₂ are attached to the meso and β positions of the ring. These positions may be unsymmetrically substituted, so that different groups R₁ₐ, R_{1b}, R_{1c}, R_{1d} are at the meso positions, etc.

FIG. 2. Depicts an array of four functional modules held at specific Cartesian coordinates relative to a surface. Intermolecular arrangement in the xy plane is defined by chemically orthogonal supramolecular directing groups; heights (z) are fixed by the rigid linkers to the ligands.

FIG. 3. Shows examples of structure directing groups (SDG) for producing 2D patterns with specific angles and edge lengths.

FIG. 4. Depicts rigid linkers for supporting ligands from 5-coordinate and 6-coordinate porphyrins and phthalocyanines.

FIG. 5. Shows examples of ligands supported above surface-binding porphyrins via rigid linkers.

FIG. 6. Approaches to synthesis of 3D nanomaterials: (A) dative bond approach and (B) covalent bond approach.

FIG. 7. Depicts packing patterns for porphyrin monolayers: (A) close-packed, (B) lamellar, and (C) functional group directed. Molecules and surface are not drawn to scale.

FIG. 8. Examples of five- and six-coordinate porphyrins reported to form monolayers on surfaces: (A) V(O)OEP, (B) Ti(O)Pc, (C) ZnPor, and (D) Rh(Cl)(Por)(py).

FIG. 9. Examples of the compounds of the present disclosure that are used as a structural unit that supports a layer of graphene above a surface or guides the orientation of carbon nanotubes, nano structures, or polymers along the surface.

### DETAILED DESCRIPTION OF THE DRAWINGS AND THE PRESENTLY PREFERRED EMBODIMENTS

The invention is described with reference to the drawings in which like elements are referred to by like numerals. The relationship and functioning of the various elements of this invention are better understood by the following detailed description. However, the embodiments of this invention as described below are by way of example only, and the invention is not limited to the embodiments illustrated in the drawings. While not intending to be bound by theory, in several of the examples below the inventors propose theories by which the invention is believed to operate. Any statements which propose a scientific theory by which an invention is believed to operate are not intended as, and should not be treated as, a limitation on the claimed invention.

When describing the compounds, compositions, methods and processes of this disclosure, the following terms have the following meanings, unless otherwise indicated.

The term “halogen” or “halo” means a chlorine, bromine, iodine, or fluorine atom.

The term “alkyl” means a hydrocarbon group that may be linear, cyclic, or branched or a combination thereof having the number of carbon atoms designated (i.e., C₁₋₈ means one to eight carbon atoms). Examples of alkyl groups include methyl, ethyl, n-propyl, isopropyl, n-butyl, t-butyl, isobutyl, sec-butyl, cyclohexyl, cyclopentyl, (cyclohexyl)methyl, cyclopropylmethyl, bicyclo[2.2.1]heptane, bicyclo[2.2.2]octane, etc. Alkyl groups can be substituted or unsubstituted, unless otherwise indicated. Examples of substituted alkyl groups include haloalkyl, thioalkyl, aminoalkyl, and the like.

The term “cycloalkyl” means an alkyl group that is cyclic.

The term “alkenyl” means a hydrocarbon group that contains at least one carbon-to-carbon double bond. The term “alkynyl” means a hydrocarbon group that contains at least one carbon-to-carbon triple bond. Alkenyl and alkynyl groups can be substituted or unsubstituted, unless otherwise indicated.

The term “aryl” means a polyunsaturated, aromatic hydrocarbon group forming a single ring (monocyclic, preferably with 6 atoms such as phenyl) or multiple rings (bicyclic (preferably with 10 atoms such as naphthyl) or polycyclic), which can be fused together or linked covalently. Examples of aryl groups include phenyl and naphthalene-1-yl, naphthalene-2-yl, biphenyl and the like. Aryl groups can be substituted or unsubstituted, unless otherwise indicated.

The term “heteroaryl” means an aromatic group containing at least one heteroatom (such as S, N, O, Si), where the heteroaryl group may be monocyclic (with preferably 5 or 6 atoms) or bicyclic (with preferably 9 or 10 atoms). Examples include pyridyl, pyridazinyl, pyrazinyl, pyrimidinyl, triazinyl, quinolinyl, quinoxalinyl, quinazolinyl, cinnolinyl, phthalazinyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiazolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, oxadiazolyl, thiadiazolyl, pyrrolyl, thiazolyl, furyl or thienyl.

The term “heterocyclyl” or “heterocyclic”, which are synonymous as used herein, means a saturated or unsaturated non-aromatic ring containing at least one heteroatom selected from nitrogen, oxygen or sulfur. The heterocyclyl ring may be monocyclic (with preferably 5 or 6 atoms) or bicyclic (with preferably 9 or 10 atoms). Examples of heterocycle groups include pyrrolidine, piperidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-dioxide, piperazine, pyran, pyridone, 3-pyrroline, thiopyran, pyrone, tetrahydrofuran, tetrahydrothiophene, quinuclidine and the like.

The term “ring” means a compound whose atoms are arranged in formulas in a cyclic form. The ring compound can be either carbocyclic or heterocyclic.

The term “carbocyclic” means a ring composed exclusively of carbon atoms.

The term “substituent” means an atom or a group that replaces another atom or group in a molecule.

The term “alkoxy” refers to —O-alkyl. Examples of an alkoxy group include methoxy, ethoxy, n-propoxy, etc.

The term “haloalkyl”, as a substituted alkyl group, refers to a monohaloalkyl or polyhaloalkyl group, most typically substituted with from 1-3 halogen atoms. Examples include 1-chloroethyl, 3-bromopropyl, trifluoromethyl and the like.

All of the above terms (e.g., “alkyl,” “aryl,” “heteroaryl” etc.), in some embodiments, include both substituted and unsubstituted forms of the indicated groups. These groups may be substituted multiple times, as chemically allowed.

This invention comprises a molecular Cartesian coordinate platform. These platforms are designed to position a set of functional molecules or molecular modules (catalysts, chromophores, luminophores, molecular magnets, molecular wires or other molecular electronics components, molecular machines) at specific Cartesian coordinates (x,y,z) relative to a planar surface, in order to achieve unique collective properties associated with their specific 3D spatial arrangement. This platform is inspired by the 3D spatial positioning of functional units by protein scaffolds found in natural systems, where the collective function of the units is often critically sensitive to the quantitative proximal relationships among subunits. For example the arrangement of the functional units of a natural system such as the photosynthetic reaction center can be reduced to the Cartesian coordinates of the units. The molecular Cartesian coordinate platform is designed to position functional units in similarly specific ways to produce similarly rich properties.

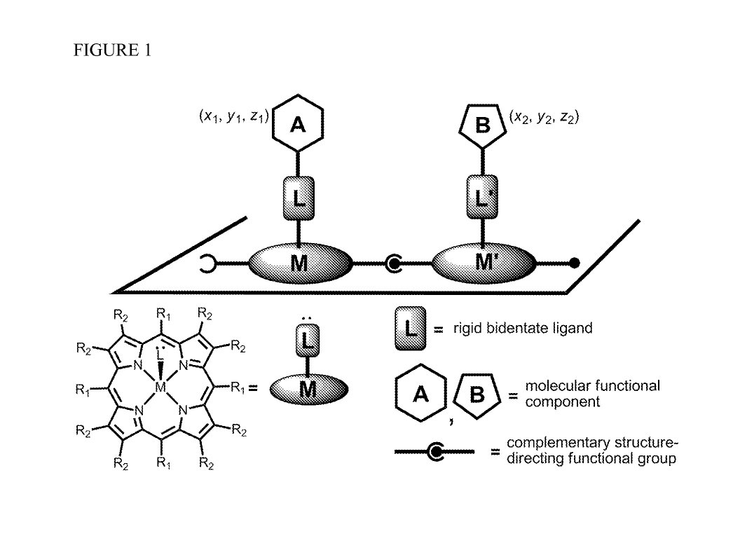

The molecular Cartesian coordinate platforms are formed from monolayers of coordination compounds coated onto planar surfaces (e.g., HOPG) under ambient laboratory conditions using solution deposition techniques. The coordination compounds possess a planar face suitable for adhesion parallel to the surface and a ligand protruding perpendicular to this face that holds the functional molecule or module. FIG. 1 is a representation of the synthetically controllable parameters in a system for supporting two functional units (A and B); this could be generalized to a larger number of units in more complex arrangements. Metalloporphyrin and metallophthalocyanine compounds are known to be capable of adopting the parallel adhesion envisioned for these systems. These molecules can be functionalized with groups at the periphery that direct the self-assembly of the molecules into a specific 2D patterned monolayer on the surface. The geometry and spacings of the pattern are controllable by changing the nature of the structure-directing peripheral groups (length of pendant arm, angular orientation of recognition group, number of groups). This pattern fixes the relative (x,y) coordinates of the modules. The heights of the modules above the surface (z coordinates) are fixed by means of a rigid linker One end of the linker is bonded covalently or datively to the surface-coating molecule. The other end may be terminated with a ligand, if functional unit A/B is a metal center, or with a covalent bond to the unit (e.g., if it is organic).

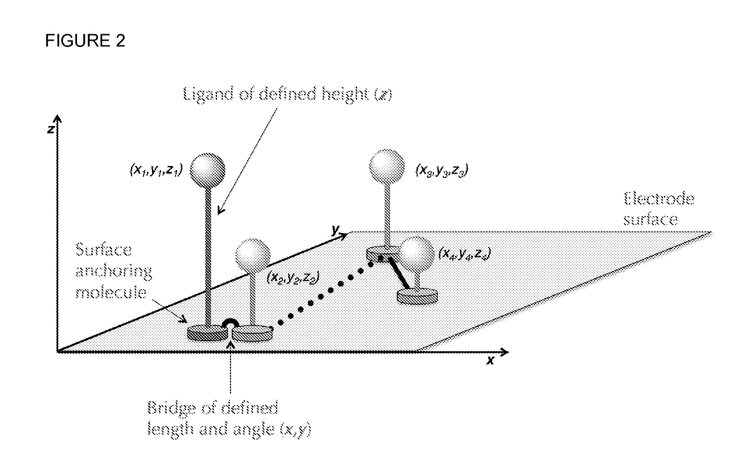

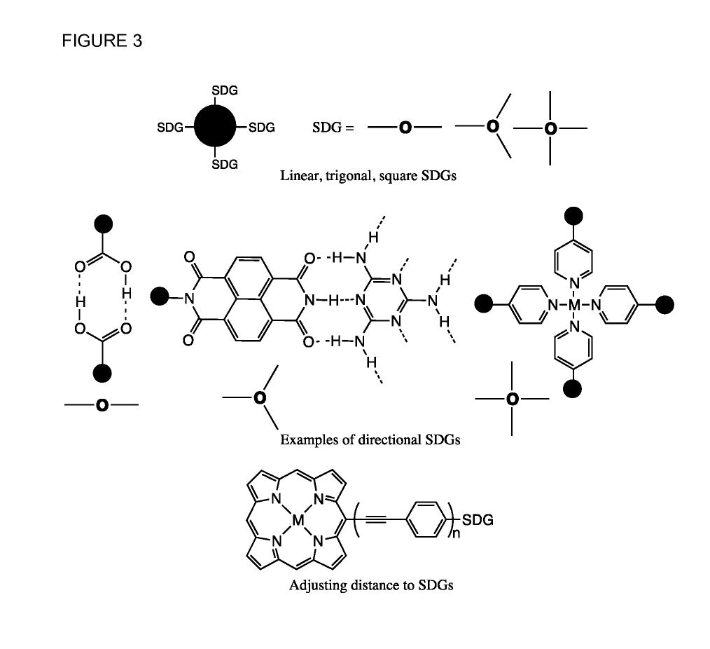

In FIG. 1 two functional units are positioned relative to each other. Additional units (C, D, . . . ) can be added by using chemically orthogonal structure-directing groups at the periphery of the supporting porphyrin/phthalocyanine, e.g., groups that specifically attach the support for C to that for A, but were incompatible with attachment to B, C, or D. In principle, patterns and arrangements of arbitrary complexity can be formed using this approach. This is represented generically in FIG. 2; distance/angle control with structure directing groups is illustrated in FIG. 3.

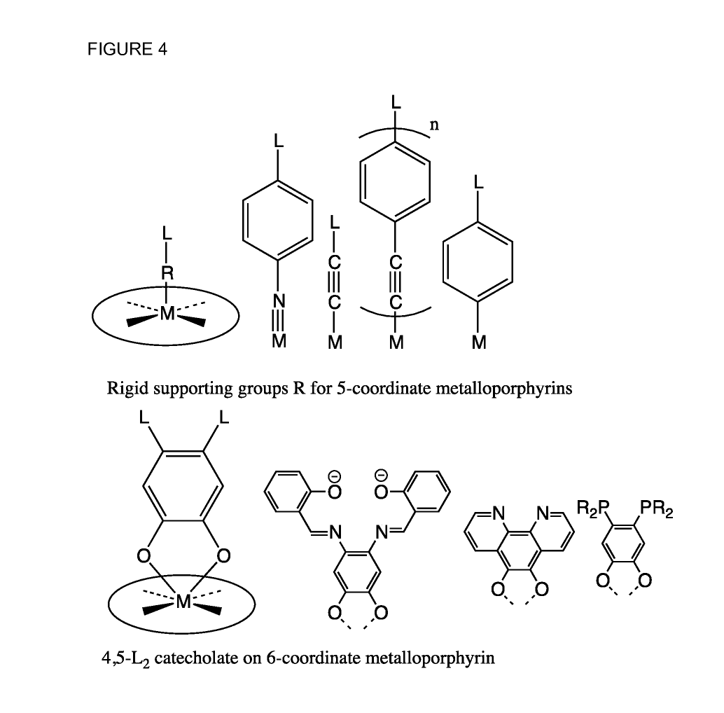

The rigid linkers that support the functional module can be of several types, as shown in FIG. 4. Use of a trivalent 5-coordinate square-pyramidal metal center (e.g., ARM), Ga(III), In(III), Mn(III), Fe(III), Co(III)) in the porphyrin/phthalocyanine can support a rigid monoanionic hydrocarbyl group (phenyl, ethynyl). Tetravalent 5-coordinate square-pyramidal metal centers (Ti(IV), Zr(IV), Hf(IV), Mo(IV)) can support the formally dianionic imido ligand NR. The ligating groups L appended to these rigid R groups can be any neutral or charged moiety compatible with the synthetic chemistry of the R group, including pyridines, amides, phosphines, NHCs, acetylides, isocyanide, cyclopentadienyl and other half-sandwich pi ligands, alkoxide, thiolate, etc. The tetravalent metal centers noted above also form 6-coordinate structures with two monoanionic ligands on the side of the porphyrin/phthalocyanine plane; the catecholate ligand binds to these two sites, and provides a rigid platform for appending chelating ligands. Examples (shown) include salen, phenanthrolene, and diphosphines.

The ligating group L can be any neutral or charged moiety compatible with the synthetic chemistry of the R group, including pyridines, amides, phosphines, NHCs, acetylides, isocyanide, cyclopentadienyl and other half-sandwich pi ligands, alkoxide, thiolate, etc.

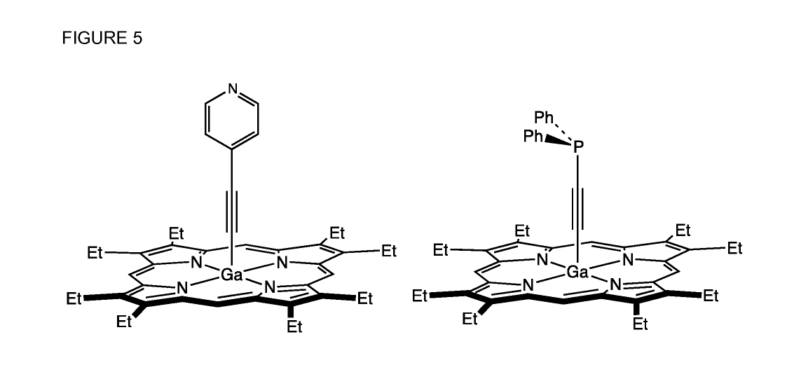

Two examples of 5-coordinate porphyrins with ligands at the end of a rigid arm have been prepared, of form Ga(OEP)(CCL) (OEP=octaethylporphyrin; L=4-pyridyl (py), PPh₂). These are shown in FIG. 5; synthetic procedures are attached. STM imaging of the pyridyl compound shows it forms an ordered 2D array with the pyridyl ligands extended perpendicular to the surface.

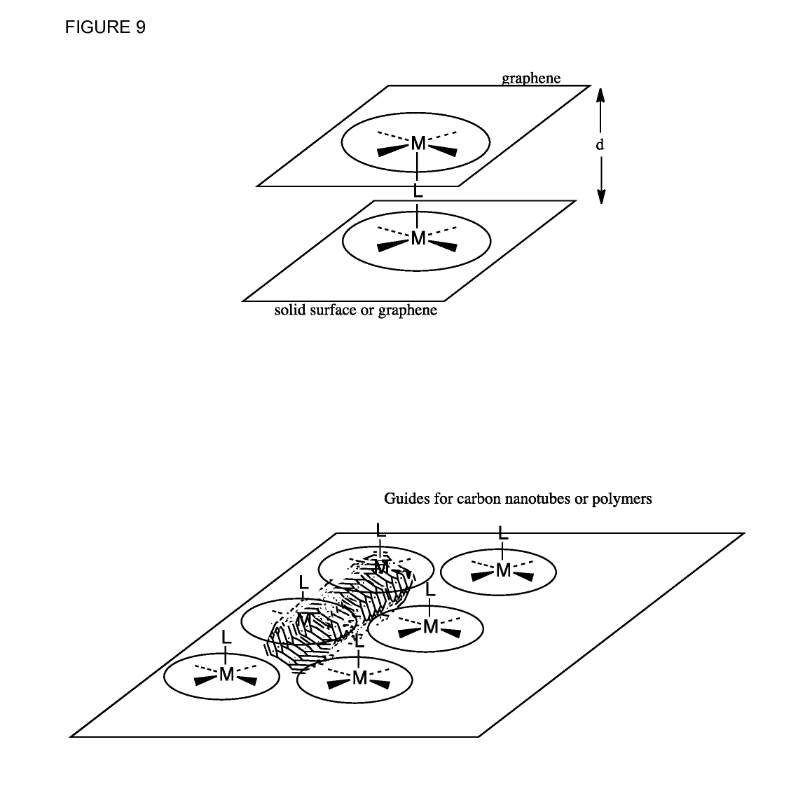

Referring to FIG. 9, the compounds of the present disclosure can also be used as a structural unit that supports a layer of graphene above a surface or guides the orientation of carbon nanotubes, nano structures, or polymers along the surface.

Exemplary Applications

Supports for multiple catalysts that carry out simultaneous, parallel, or sequential processes.

Allen and MacMillan (Chem Sci 2012, 3, 633) concisely summarize classes of dual-catalyst systems based on their transformations. As described therein, embodiments of the present invention may be used to carry out, for example, bifunctional catalysis, double activation catalysis, cascade catalysis and synergistic catalysis. The molecular Cartesian coordinate platform is useful for these types of catalysts in several ways: (a) it can isolate incompatible catalysts from each other, e.g., those that degrade via in bimolecular processes (dimerization, ligand exchange); (b) it can serve as an “assembly lines” where the product of one catalyst is passed off to an adjacent catalysts, or where two catalysts carry out simultaneous transformations on two portions of a long substrate.

Molecular Magnet Arrays

A number of groups are attempting to fabricate arrays of single-molecule magnets (SMM) or paramagnetic compounds for applications in magnetic storage, spintronics, quantum computing, and sensing. A paper by Cavallini (Phys Chem Chem Phys 2008, 10, 757) describes the details of patterning. The methods described by others are low resolution compared with that provided by the Cartesian platform. An example of SMMs tethered to a metal surface (flexible tether, no x,y spatial control) was recently published in Nature (Mannini, et al., 2010, 468, 417). Embodiments of the present invention can comprise similar elements.

“Breadboards” for molecular electronics circuits and for optical light paths.

There are numerous applications envisioned for molecular-length-scale circuitry that transmits electrons or photons. The molecular Cartesian coordinate platform provides a way to organize the components of these circuits. A recent example of a “photonic wire” (JACS 2011, 133, 4193) uses a series of Førster Resonance Energy Transfer (FRET) luminophores as the photochemical relays, and “DNA origami” as the support. In the Cartesian platform, FRET groups can be organized at more precisely controlled distances and angles. Excitation of the short-wavelength-emitting luminophore initiates a series of energy transfer events that can move photon energy along a designed path (the photon wire). Similarly, there are not good ways of organizing electron-tunneling molecular wires to achieve the function of circuits. Most experiments study the properties of single wires that span a gap in a metallic wire (break junction). Attempts to organize wires into more complex circuitry has typically made the wires conform to the shape of the support, e.g., a virus coated with gold nanoparticles (Small 2005, 1, 702). In the Cartesian platform, the ligands can be used to attach metal centers or small nanoclusters with an affinity for the terminal groups of the molecular wires.

Square-pyramidal molecules are building blocks for the assembly of three dimensional (3D) self-assembled nanomaterials on solid supports. The compounds used to develop this concept are metalloporphyrins, which have been shown to self-assemble into ordered 2D monolayers on a variety of substrates with the molecular plane lying parallel to the surface. Nearly all prior examples of these overlayers have been constructed from four-coordinate porphyrins. A monolayer comprised of metalloporphyrins with five-coordinate metal centers presents axial ligands that point up from the surface. If the axial ligands are bidentate, they can act as binding posts for functional components, which can then be patterned according to the structure encoded into the self-assembled porphyrin layer. This concept is shown in FIG. 1, where two functional components are positioned in the z direction by ligands of a given height, and in the x,y space by porphyrin-edge functional groups of a given length.

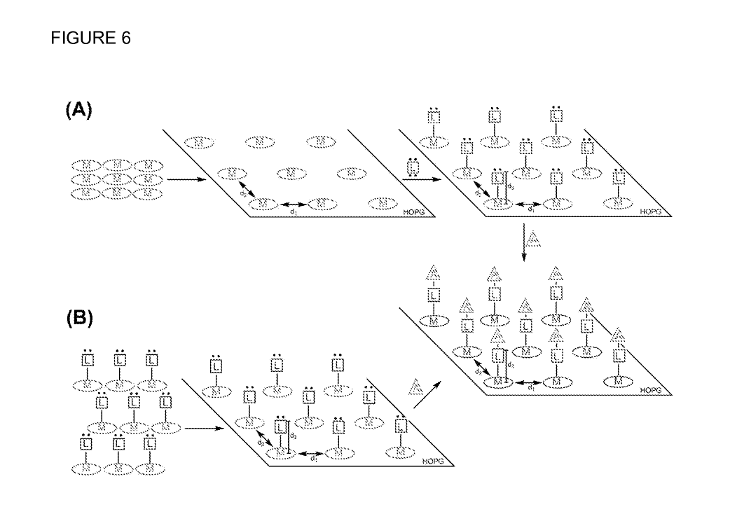

One aspect of the invention is the solution-phase synthesis of close-packed 2D arrays of porphyrin molecules with orthogonal ligands to enable access to 3D nanomaterials. Literature examples of monolayers composed of simple four-coordinate porphyrin molecules were used as a starting point for development of five-coordinate metalloporphyrin monolayers. Two approaches that were used in attempts to synthesize five-coordinate metalloporphyrin monolayers are presented in FIG. 6; a dative bond approach (FIG. 6(A)) where a four-coordinate metalloporphyrin monolayer is formed on the substrate and a ligand is dosed onto the monolayer where it can bind to the metal; and a covalent bond approach where the five-coordinate porphyrin is synthesized prior to deposition (FIG. 6(B)). The variables d₁ and d₂ are governed by the substituents at the periphery of the porphyrin heterocycle and d₃ is governed by the height of the ligand. For both approaches presented in FIG. 6, the examples of bidentate ligand are electron pair donors, which can bind functional component that are electron pair acceptors.

To realize assemblies of the type shown in FIG. 6, decisions must be made regarding the environmental conditions for depositing the monolayer, the surface to be functionalized, the structure of the porphyrins to be deposited, the choice of metal and ligand, and the experimental probes to be used to deduce the composition and structure of the monolayer.

The environmental conditions under which monolayers are deposited and characterized primarily fall into two categories: ultra high vacuum (UHV) and ambient environments. UHV conditions allow detailed characterization of monolayers over a wide range of temperatures with a wide variety of sophisticated instrumentation. However, deposition is performed by sublimation, which requires volatile components, and the substrate preparation and deposition process is slow. Under ambient conditions, monolayers are primarily deposited onto substrates from solution, which allows faster screening, but characterization in air or liquid limits the spatial resolution of imaging tools, choice of substrates, and the types of spectroscopic probes that can be used. Ambient conditions were chosen for the deposition and characterization experiments because it is more compatible with high-throughput testing of concepts for FIG. 6.

The choice of surface to be functionalized and the conditions for deposition are closely linked. Commonly used surfaces for constructing porphyrin monolayers fall into two main categories: surfaces that are used in ambient conditions, such as highly oriented pyrolytic graphite (HOPG) and Au(111), and those that are used in UHV, such as crystals of other noble metals. HOPG is preferable over Au(111) as the substrate for deposition of porphyrin monolayers for certain embodiments because it is more easily cleaned and reproducibly planar, which allows for rapid synthesis.

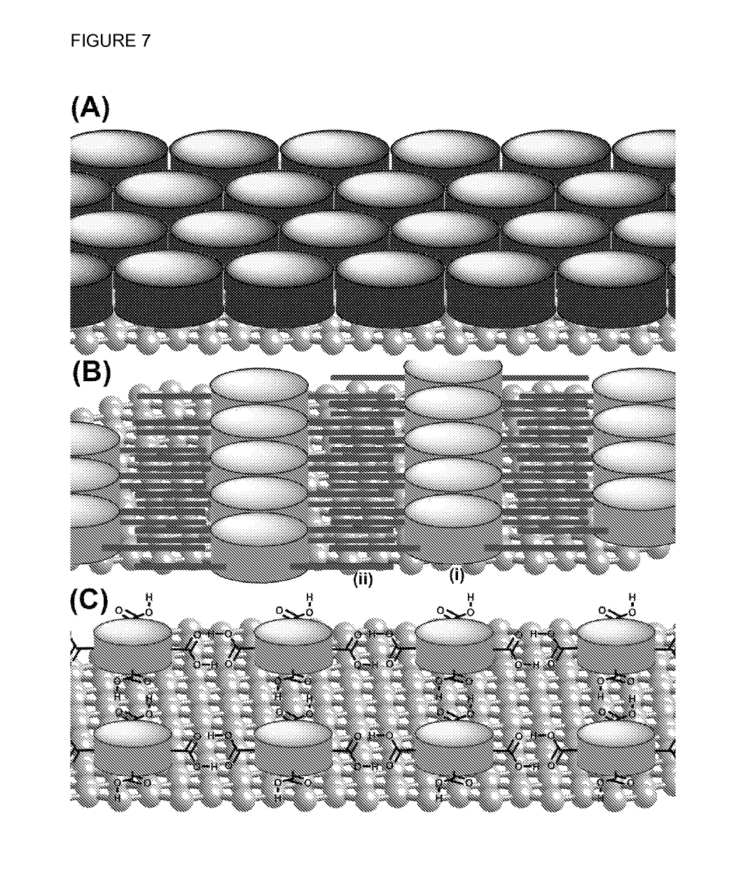

The choice of the porphyrin to be deposited determines the 2D ordering of the monolayer, d₁ and d₂ (FIG. 6). The forces governing 2D assemblies on solid substrates can be divided into two categories: molecule-substrate interactions and molecule-molecule interactions. Although molecule-substrate interactions play an important role in the adsorption process, for HOPG, which is considered an inert surface, these forces are primarily limited dispersion forces between the adsorbate and the substrate, and minimally perturb the molecular properties of the adsorbate. The molecule-molecule interactions that govern the self-assembly of organic molecules on surfaces fall in to a variety of classes, including van der Waals (vdW) forces, hydrogen bonds, and metal-ligand bonds. All three of these types of forces can direct the assembly of molecules in 2D, and metal-ligand bonding has the added advantage that it can be used to direct 3D assemblies. Examples of each of these interactions governing the 2D assemblies of porphyrin and phthalocyanine monolayers have been reported, for example in Barth, J. V., Surf Sci. 2009, 603 (10-12), 1533-1541; Mohnani, S; Bonifazi, D., Coord. Chem. Rev. 2010, 254 (19-20), 2342-2362; De Feyter, S; De Schryver, F. C., Chem. Soc. Rev. 2003, 32 (3), 139-150; Rosei, F; Schunack, M; Naitoh, Y; Jiang, P; Gourdon, A; Laegsgaard, E; Stensgaard, I; Joachim, C; Besenbacher, F., Prog. Surf Sci. 2003, 71 (5-8), 95-146; Yoshimoto, S; Itaya, K., J. Porphyrins Phthalocyanines 2007, 11 (5-6), 313-333; Otsuki, J., Coord. Chem. Rev. 2010, 254 (19-20), 2311-2341; and Gottfried, J. M; Marbach, H., Z. Phys. Chem. 2009, 223 (1-2), 53-74, all of which are incorporated by reference in their entirety. Monolayers of porphyrins with small alkyl substituents such as octaethylporphyrins and phthalocyanines, which have a disk-like shape, tend to pack tightly with little space between heterocyclic cores (FIG. 7(A)). As the alkyl chains are lengthened, the spacing between the heterocycles becomes larger. With sufficiently long alkyl chains, the packing geometry changes to a lamellar structure (FIG. 7(B)). These monolayers have two distinctly different phases: (i) a phase where porphyrin rings are tightly packed into rows with a large space between the rows and (ii) a lamellar phase where the long alkyl chains from porphyrins that are directly opposite each other in adjacent rows interdigitate to maximize the vdW forces along the alkyl chains, which directs the ordering. A third type of ordering is observed for monolayers of porphyrins with directing functionalities incorporated into the periphery of the heterocycle, such as hydrogen bonding moieties like carboxyl groups and pyridyl groups (FIG. 7(C)). Monolayers with such functionalities tend to be less tightly packed and possess geometries that are strongly governed by the interactions between adjacent functional groups. These monolayers are good examples of using functional groups to precisely control distances and geometries of adsorbates.

Exemplary heterocyclic systems that can be adapted for use in the present invention are shown in Tables 1-9.

TABLE 1
Exemplary porphyrin derivatives that can be modified for use with the present invention

| | Coordination | Bond | | | |
|---|---|---|---|---|---|
| Metal | Number | order | Axial R | Note | Source |
| Al(III) | 5 | 1 | O | Bridging dimer | Porphyrin handbook, Vol 12, Chapter 53, Ref. 266 |
| Co(III) | 5 | 1 | —CH2CH3 | | Porphyrin Handbook, Vol. 6, Chapter 29, Table 8 |
| Co(III) | 5 (6) | 1 | —CH2CH3 | Dative bond with Py | Porphyrin Handbook, Vol. 6, Chapter 29, Table 8 |
| Co(III) | 5 | 1 | methyl, | | Porphyrin Handbook, Vol. 6, Chapter 29 Ref. 948 |
| | | | ethyl, | | |
| | | | propyl, | | |
| | | | butyl | | |
| Co(III) | 5 | 1 | H− | Transient species in | Porphyrin Handbook, Vol. 6, Chapter 29 Ref. 951 |
| | | | | radical reaction | |
| Co(III) | 5 | 1 | —CH(OAc)CH3, | | Porphyrin Handbook, Vol. 6, Chapter 29 Ref. 951 |
| | | | —CH(OAc)C(CH3)2CN | | |
| Co(III) | 5 | 1 | Cl | | Porphyrin handbook, Vol 12, Chapter 53, Ref. 289-290 |
| Cr(III) | 5 | 1 | Cl− | | Porphyrin Handbook, Vol. 6, Chapter 29, Ref. 94 |
| Cr(III) | 5 | 1 | —OPh | | Porphyrin Handbook, Vol. 6, Chapter 29 Ref. 165 |
| Dy(III) | 5 | 1 | OH | | Porphyrin handbook, Vol 12, Chapter 53, Ref. 258 |
| Dy(III) | “6” | 1 + | acac | | Porphyrin handbook, Vol 12, Chapter 53, Ref. 267 |
| | | dative | | | |
| Er(III) | 5 | 1 | OH | | Porphyrin handbook, Vol 12, Chapter 53, Ref. 258 |
| Er(III) | “6” | 1 + | acac | | Porphyrin handbook, Vol 12, Chapter 53, Ref. 269 |
| | | dative | | | |
| Fe(II) | 5 | 1 | —CH2CH3, | Negatively charged | Porphyrin Handbook, Vol. 6, Chapter 29, Ref. 243, 244 |
| | | | —CH2CH2CH3 | complexes | |
| Fe(III) | 5 (6) | 1 | Cl− | Additional dative | Porphyrin Handbook, Vol. 1, Chapter 4, Ref. 283 |
| | | | | interaction with | |
| | | | | additional nitrogenous | |
| | | | | base | |
| Fe(III) | 5 | 1 | ClO₄⁻ | Weakly coordinating | Porphyrin Handbook, Vol. 6, Chapter 29, Ref. 15, 393-400 |
| Fe(III) | 5 | 1 | OO²⁻ | Negatively charged | Porphyrin Handbook, Vol. 6, Chapter 29 Pg. 108 |
| | | | | complex | |
| Fe(III) | 5 | 1 | OH− | | Porphyrin Handbook, Vol. 6, Chapter 29 Pg. 108 |
| Fe(III) | 5 | 1 | OR− | | Porphyrin Handbook, Vol. 6, Chapter 29 Pg. 108 |
| Fe(III) | 5 | 1 | ROO− | | Porphyrin Handbook, Vol. 6, Chapter 29 Pg. 108 |
| Fe(III) | 5 | 1 | N₃⁻ | | Porphyrin Handbook, Vol. 6, Chapter 29 Pg. 108 |
| Fe(III) | 5 | 1 | RC₂⁻ | Acetylide compound | Porphyrin Handbook, Vol. 6, Chapter 29 Pg. 108 |
| Fe(III) | 5 | 1 | NCS− | | Porphyrin Handbook, Vol. 6, Chapter 29 Pg. 108 |
| Fe(III) | 5 | 1 | RCOO− | | Porphyrin Handbook, Vol. 6, Chapter 29 Pg. 108 |
| Fe(III) | “6” | ? | NO₃⁻ | Bidentate | Porphyrin Handbook, Vol. 6, Chapter 29 Pg. 108 |
| Fe(III) | 5 | 1 | OTeF₅⁻ | | Porphyrin Handbook, Vol. 6, Chapter 29 Pg. 108 |
| Fe(III) | 5 | 1 | SO₄²⁻ | Dimer | Porphyrin Handbook, Vol. 6, Chapter 29 Pg. 108 |
| Fe(III) | 5 | 1 | HSO₄⁻ | | Porphyrin Handbook, Vol. 6, Chapter 29 Pg. 108 |
| Fe(III) | 5 | 1 | RSO₃⁻ | | Porphyrin Handbook, Vol. 6, Chapter 29 Pg. 108 |
| Fe(III) | 5 | 1 | WS₄²⁻ | | Porphyrin Handbook, Vol. 6, Chapter 29 Pg. 108 |
| Fe(III) | 5 | 1 | trazolate | | Porphyrin Handbook, Vol. 6, Chapter 29 Pg. 108 |
| Fe(III) | 5 | 1 | tetrazolate | | Porphyrin Handbook, Vol. 6, Chapter 29 Pg. 108 |
| Fe(III) | 5 | 1 | —OOtBu | | Porphyrin Handbook, Vol. 6, Chapter 29 Pg. 110 |
| Fe(III) | 5 | 1 | CF₃SO₃⁻ | Weakly coordinating | Porphyrin Handbook, Vol. 6, Chapter 29 Ref. 374 |
| Fe(III) | 5 | 1 | SbF₆⁻ | Weakly coordinating | Porphyrin Handbook, Vol. 6, Chapter 29 Ref. 400-402 |
| Fe(III) | 5 | 1 | C(CN)₃⁻ | Weakly coordinating | Porphyrin Handbook, Vol. 6, Chapter 29 Ref. 374, 403 |
| Fe(III) | 5 | 1 | CF₃CO₂⁻ | Weakly coordinating | Porphyrin Handbook, Vol. 6, Chapter 29 Ref. 374, 400 |
| Fe(III) | 6 | 1,1 | CN⁻ | Anionic | Porphyrin Handbook, Vol. 6, Chapter 29 Ref. 440, 414, 214 |
| Fe(III) | 5 (6) | 1 | CN⁻ | Dative bond with Py | Porphyrin Handbook, Vol. 6, Chapter 29 Ref. 440 |
| Fe(III) | 6 | 1,1 | Im⁻, CN⁻ | Anionic | Porphyrin Handbook, Vol. 6, Chapter 29 Ref. 489 |
| Fe(III) | 6 | 1,1 | Im⁻ | Anionic | Porphyrin Handbook, Vol. 6, Chapter 29 Ref. 490 |
| Fe(III) | 5 (6) | 1 | ⁻CCR | Dative bond with Py | Porphyrin Handbook, Vol. 6, Chapter 29 Ref. 330 |
| Fe(III) | 5 (6) | 1 | ⁻OR | Dative bond with ImH | Porphyrin Handbook, Vol. 6, Chapter 29 Ref. 331 |
| Fe(III) | 5 (6) | 1 | p- | Dative bond with ImH | Porphyrin Handbook, Vol. 6, Chapter 29 Ref. 331 |
| | | | NO₂C₆H₄O⁻ | | |
| Fe(III) | 5 (6) | 1 | aryl, | Dative bond with ImH | Porphyrin Handbook, Vol. 6, Chapter 29 Ref. 516 |
| | | | alkyl, | or Py | |
| | | | carbanion | | |
| Fe(III) | 5 (6) | 1 | alkyl, | Some with dative | Porphyrin Handbook, Vol. 6, Chapter 29 Table 13 |
| | | | aryl, silyl | bond with Py, NMeIm | |
| Fe(III) | 5 | 1 | Cl | | Porphyrin handbook, Vol 7, Chapter 32, Ref. 32-35, 36, |
| | | | | | 137-139, 157-158, 180-181 |
| Fe(III) | 5 | 1 | OCH3 | | Porphyrin handbook, Vol 7, Chapter 32, Ref. 32-35, 137-139 |
| Fe(III) | 5 | 1 | ClO4 | | Porphyrin handbook, Vol 7, Chapter 32, Ref. 36, 140-147, |
| | | | | | 158-159, 180-181 |
| Fe(III) | 5 | 1 | C(CN)3 | | Porphyrin handbook, Vol 7, Chapter 32, Ref. 36, 148-149, |
| | | | | | 158-159 |
| Fe(III) | 5 | 1 | SbF6 | | Porphyrin handbook, Vol 7, Chapter 32, Ref. 36, 149, 158-159 |
| Fe(III) | 5 | 1 | Br | | Porphyrin handbook, Vol 7, Chapter 32, Ref. 36, 149, 158-159, |
| | | | | | 180-181 |
| Fe(III) | 5 | 1 | I | | Porphyrin handbook, Vol 7, Chapter 32, Ref. 36, 158-159, |
| | | | | | 180-181 |
| Fe(III) | 5 | 1 | OTeF3 | | Porphyrin handbook, Vol 7, Chapter 32, Ref. 36, 158-159 |
| Fe(III) | 5 | 1 | ReO4 | | Porphyrin handbook, Vol 7, Chapter 32, Ref. 36, 158-159 |
| Fe(III) | 5 | 1 | ONC(CN)2 | | Porphyrin handbook, Vol 7, Chapter 32, Ref. 36, 158-159 |
| Fe(III) | 5 | 1 | BF4 | | Porphyrin handbook, Vol 7, Chapter 32, Ref. 36, 158-159 |
| Fe(III) | 5 | 1 | CF3SO3 | | Porphyrin handbook, Vol 7, Chapter 32, Ref. 36, 158-159 |
| Fe(III) | 5 | 1 | AsF6 | | Porphyrin handbook, Vol 7, Chapter 32, Ref. 36, 158-159 |
| Fe(III) | 5 | 1 | Co(C2B9H11)2 | | Porphyrin handbook, Vol 7, Chapter 32, Ref. 36, 158-159 |
| Fe(III) | 5 | 1 | CB11H12 | | Porphyrin handbook, Vol 7, Chapter 32, Ref. 36, 158-159 |
| Fe(III) | 5 | 1 | Ag(Br6CB11H6)2 | | Porphyrin handbook, Vol 7, Chapter 32, Ref. 36, 158-159 |
| Fe(III) | 5 | 1 | F | | Porphyrin handbook, Vol 7, Chapter 32, Ref. 165-166, |
| | | | | | 180-181 |
| Fe(III) | 5 | 1 | H2O | | Porphyrin handbook, Vol 7, Chapter 32, Ref. 101 |
| Fe(III) | 5 | 1 | N3 | | Porphyrin handbook, Vol 7, Chapter 32, Ref. 154-155 |
| Fe(III) | 6 | 1,1 | F | | Porphyrin handbook, Vol 7, Chapter 32, Ref. 165-166 |
| Fe(III) | 5 | 1 | O | Bridging dimer | Porphyrin handbook, Vol 12, Chapter 53, Ref. 224, 265 |
| Fe(IV) | 5 | 2 | O | | Porphyrin Handbook, Vol. 6, Chapter 29 Pg. 110 |
| Fe(IV) | 6 | 1,1 | CH₃O⁻ | | Porphyrin Handbook, Vol. 6, Chapter 29 Ref. 832 |
| Fe(IV) | 6 | 1,1 | Ph⁻, Br⁻ | | Porphyrin Handbook, Vol. 6, Chapter 29 Ref. 157 |
| Fe(IV) | 5 | 2 | O | | Porphyrin handbook, Vol 7, Chapter 32, Ref. 231, 253, |
| | | | | | 258-272 |
| Fe(IV) | 6 | 1,1 | OMe | | Porphyrin handbook, Vol 7, Chapter 32, Ref. 253 |
| Fe(IV) | 6 | 2,1 | O, | pi-cation radical | Porphyrin handbook, Vol 11, Chapter 44, Ref. 97 |
| | | | NCCH3 | | |
| Fe(IV) | 6 | 2,1 | O, Cl | pi-cation radical | Porphyrin handbook, Vol 11, Chapter 44, Ref. 97 |
| Fe(V) | 6 | 2,1 | O, F | | Porphyrin handbook, Vol 7, Chapter 32, Ref. 282 |
| Ga(III) | 5 | 1 | alkyl, | | Balch, A. L., et al. Inorganica Chimica Acta, 205 (1993) |
| | | | alkoxy, | | 137-143 |
| | | | alkylperoxy | | |
| Ga(III) | 5 | 1 | Cl−, F−, I− | | Coutsolelos, A., Guilard, R. Polyhedron Vol. 5, No. 6, pp. |
| | | | | | 1157-1 164.1986 |
| Ga(III) | 5 | 1 | alkyl, | | Kadish, K. M., et al. Inorg. Chem. 1985, 24, 4521-4528 |
| | | | alkenyl, | | |
| | | | CCPh | | |
| Ga(III) | 5 | 1 | alkyl, | | Kadish, K. M., et al. Inorg. Chem. 1989, 28, 2518-2523 |
| | | | alkenyl, | | |
| | | | CCPh | | |
| Ga(III) | 5 | 1 | Cl−, OAc−, | | Kadish, K. M., et al. Inorg. Chem. 1987, 26, 4167-4173 |
| | | | F− | | |
| Ga(III) | 5 (6) | 1 | OH− | Dative bond with H2O | Kadish, K. M., et al. Inorg. Chem. 1987, 26, 4167-4173 |
| Ga(III) | 5 | 1 | Br− | | Parzuchowski, P. G., et al. Inorganica Chimica Acta |
| | | | | | (2003), 355, 302-313. |
| Gd(III) | “6” | 1 + | acac | | Porphyrin handbook, Vol 12, Chapter 53, Ref. 265 |
| | | dative | | | |
| Ge(IV) | 6 | 1,1 | ferrocenyl | | Porphyrin Handbook, Vol. 3, Chapter 14, Ref. 32-34 |
| Ho(III) | “6” | 1 + | acac | | Porphyrin handbook, Vol 12, Chapter 53, Ref. 268 |
| | | dative | | | |
| In(III) | 5 | 1 | alkyl, | | Kadish, K. M., et al. Inorg. Chem. 1989, 28, 2518-2523 |
| | | | alkenyl, | | |
| | | | CCPh | | |
| Ln(III) | “6” | 1 + | acac | | Porphyrin handbook, Vol 12, Chapter 53, Ref. 264 |
| | | dative | | | |
| Mg(II) | 5, 6 | Dative | OR | | Porphyrin Handbook, Vol. 1, Chapter 3 |
| Mn(III) | 5 | 1 | Cl− | | Porphyrin Handbook, Vol. 1, Chapter 1, Ref. 317 |
| Mn(III) | 6 | 1,1 | CN− | Anionic | Porphyrin Handbook, Vol. 6, Chapter 29 Pg. 280 |
| Mn(III) | 5 | 1 | Cl | | Porphyrin handbook, Vol 11, Chapter 47, Ref. 130-131 |
| Mn(IV) | 6 | 1,1 | Cl−, —OCH3 | | Porphyrin Handbook, Vol. 6, Chapter 29 Ref. 376, 377, 985 |
| Mn(IV) | 5 | 2 | O | | Porphyrin Handbook, Vol. 6, Chapter 29 Ref. 229 |
| Mn(IV) | 6 | 1,1 | NCR | | Porphyrin handbook, Vol 12, Chapter 53, Ref. 227 |
| Rh(III) | 5 (6) | 1 | Cl− | Additional dative | Porphyrin Handbook, Vol. 1, Chapter 1, Ref. 325 |
| | | | | interaction with | |
| | | | | additional nitrogenous | |
| | | | | base | |
| Rh(III) | 5 | 1 | I− | | Porphyrin Handbook, Vol. 6, Chapter 29, Table 8 |
| Rh(III) | 5 | 1 | Me− | | Porphyrin Handbook, Vol. 6, Chapter 29, Table 8 |
| Rh(III) | 5 | 1 | H− | | Porphyrin Handbook, Vol. 6, Chapter 29, Table 8 |
| Rh(III) | 5 | 1 | I | | Porphyrin handbook, Vol 11, Chapter 49, Ref. 160 |
| Ru(II) | 6 | Dative | CO, NR | | Porphyrin Handbook, Vol. 1, Chapter 4, Ref. 135-137 |
| Ru(II) | 5 | 1 | H− | Negatively charged | Porphyrin Handbook, Vol. 6, Chapter 29, Table 8 |
| | | | | complex | |
| Ru(II) | 6 | 1,1 | NO+, Cl− | | Porphyrin Handbook, Vol. 6, Chapter 29, Table 8 |
| Ru(IV) | 6 | 1,1 | Me− | | Porphyrin Handbook, Vol. 6, Chapter 29, Table 8 |
| Ru(IV) | 5 | 2 | —CHCH3 | | Porphyrin Handbook, Vol. 6, Chapter 29, Table 8 |
| Sb(V) | 5 | 2 | O | | Porphyrin handbook, Vol 11, Chapter 50, Ref. 22 |
| Sn(III) | 5 | 1 | Cl | | Porphyrin handbook, Vol 11, Chapter 49, Ref. 160 |
| Sn(IV) | 6 | 1,1 | —OR | Distorted Octahedral | Porphyrin Handbook, Vol. 1, Chapter 4, Ref. 168 |
| Sn(IV) | 6 | 1,1 | 2 OH | | Porphyrin handbook, Vol 11, Chapter 50, Ref. 19-21, 26 |
| Sn(IV) | 6 | 1,1 | 2 Cl | | Porphyrin handbook, Vol 11, Chapter 50, Ref. 24 |
| Tb(III) | 5 | 1 | OH | | Porphyrin handbook, Vol 12, Chapter 53, Ref. 258 |
| Tb(III) | “6” | 1 + | acac | | Porphyrin handbook, Vol 12, Chapter 53, Ref. 266 |
| | | dative | | | |
| Yb(III) | 5 | 1 | OH | | Porphyrin handbook, Vol 12, Chapter 53, Ref. 258 |
| Zn(II) | 5 | Dative | NR, OR | | Numerous |
| Zr(IV) | 6 | 1,1 | Cl | | Brand and Arnold, 1993 |
| Zr(IV) | 6 | 1,1 | OTf | | Brand and Arnold, 1993 |
| Zr(IV) | 6 | 1,1 | OAc | | Brand and Arnold, 1993, J. W. Buehler et al., and others |
| Zr(IV) | 6 | 1,1 | OR | | Brand and Arnold, 1993 |
| Zr(IV) | 6 | 1,1 | R | | Brand and Arnold, 1993 |
| Zr(IV) | 6 | 1,1 | Aryl | | Brand and Arnold, 1993 |
| Zr(IV) | 6 | 1,1 | Cycloocta | Bidentate | Brand and Arnold, 1993 |
| | | | tetraenyl | | |
| Mo(IV) | 5 | 2 | NR | | Berraeu, L. M., et al. Inorg. Chem. 2005, 44, 7304-7306 |
| Ti(IV) | 5 | 2 | NR | Intermetal imido | Berraeu, L. M., et al. Inorg. Chem. 2005, 44, 7304-7306 |
| | | | | transfer from Mo to Ti | |
| Te(IV) | 6 | 1,1 | Cl− | cis Cl−; unstable in the | Grubisha, D. S., et al. Angew. Chem. Int. Ed. 2001, 40, No. |
| | | | | presence of light and | 24 |
| | | | | in air | |
| Ti(IV) | 6 | 1,1 | catacholate | reaction of Ti(II) | Du, G., et al. Organometallics 2004, 23, 4230-4235 |
| | | | | complex with | |
| | | | | aldehydes | |
| Ti(IV) | 5 | 2 | NNR2, | highly reactive with | Thorman, J. L., et al. Inorg. Chem. 2000, 39, 1301-1304 |
| | | | NR | oxygenated species | |
| Zr(IV) | 5 | 2 | NR | | Thorman, J. L., et al. Inorg. Chem. 1999, 38, 3814-3824 |
| Hf(IV) | 5 | 2 | NR | | Thorman, J. L., et al. Inorg. Chem. 1999, 38, 3814-3824 |
| Zr(IV) | 6 | 1,1 | N, O | Bidentate | Thorman, J. L., et al. Inorg. Chem. 1999, 38, 3814-3824 |
| Zr(IV) | 6 | 1,1 | N, O | Bidentate | Thorman, J. L., et al. Inorg. Chem. 1999, 38, 3814-3824 |
| Mo(IV) | 5 | 2 | S, Se | | Berraeu, L. M.,et al. Inorg. Chem. 1995, 34, 3485-3490 |
| Os(II) | 5 | 2 | CRR′ | | Woo, L. K., et al. Organometallics 1992, 11, 2344-2346 |
| Os(II) | 5 | 2 | SiRR′ | | Woo, L. K., et al. Organometallics 1991, 10, 3977-3982 |
| Mn(V) | 5 | 3 | N | | Woo, L. K., et al. J. Am. Chem. SOC. 1991, 113, 8478-8484 |
| Ru(IV) | 5 | 2 | CRR′ | | Che, C-M, et al. J. Am. Chem. Soc. 2001, 123, 4119-4129 |
| Fe(II) | 5 | 2 | CRR′, | air and light sensitive | Ziegler, C. J., et al. Journal of Organometallic Chemistry |
| | | | CCRR′ | | 528 (1997) 83-90 |
| Mn(V) | 5 | 2 | CRR′ | | Jones, T. K., et al. Inorganica Chimica Acfa, 206 (1993) 5-7 |
| Ru(II) | 5 | 2 | CRR′ | air-stable | Wada, S., et al. Acta Cryst. (2003). C59, m369-m370 |
| Os(III) | 6 | 2,1 | CRR′, O | oxo-bridged Os- | K. Sawano et al. Inorganica Chimica Acta 358 (2005) |
| | | | | carbene porphyrin | 1830-1834 |
| | | | | dimer | |
| Cr(V) | 5 | 3 | N | | Campochiaro, C., et al. Inorg. Chem. 1985, 24, 449-450 |
| | | | | | 449 |
| Ru(II) | 6 | 1,1 | NO, | nitrido-bridged | Yeung, W-H, et al. Inorgan ca Chimica Acta 290 (1999) |
| | | | NOs(VIII) | bimetallic complexes | 28-35 |
| | | | or | | |
| | | | NOs(VI) | | |
| Re(V) | 5 | 3 | N:Cr(III)PorCl | | Tong, C., et al. Inorg. Chem. 1996, 35, 5108-5109 |
| Nb(V) | 7 | 1,1,1 | O | NbPor dimer with 3 | Watanabe Y., et al. Structure and Bonding (Berlin) (2000), |
| | | | | bridging oxos | 97 (Metal-Oxo and Metal-Peroxo Species in Catalytic |
| | | | | | Oxidations), 61-89. |
| Fe(III) | 5 | 1 | O | mu-oxo dimer | Suzuki, M., et al. Chemistry Letters Vol. 32, No. 7 (2003) |
| Fe(III) | 5 | 1 | O, OH | mu-oxo and mu- | Nanthakumar, A., et al. J. Am. Chem. SOC. 1993, 115, |
| | | | | hydroxy bridged with | 8513-8514 8513 |
| | | | | Cu(II); cationic | |
| Zr(IV) | 7 | ? | OH | ZrPor dimer bridged | Kim, H-J, et al. Chemistry Letters Vol. 22 (1993), No. 5 |
| | | | | by 3 OH−; cationic | p. 807 |
| Zr(IV) | 7 | 1,1,dative | O, OH | ZrPor dimer bridged | Kim, H-J, et al. Chemistry Letters Vol. 22 (1993), No. 5 |
| | | | | by 2 OH− and O²⁻ | p. 807 |
| Ru(IV) | 6 | 1,1 | O, X | oxo-bridged; Cl, Br, | Sugimoto, H., et al. Bulletin of the Chemical Society of |
| | | | | OAc as other axial | Japan (1982), 55(3), 822-8. |
| | | | | ligand | |
| Os(IV) | 6 | 1,1 | O, X | oxo-bridged; Cl, Br, | Sugimoto, H., et al. Bulletin of the Chemical Society of |
| | | | | OAc as other axial | Japan (1982), 55(3), 822-8. |
| | | | | ligand | |
| Sc(III) | 5 | 1 | O | oxo-bridged dimer | Leenstra, W. R., et al. Journal of Chemical Physics (1979), |
| | | | | | 71(8), 3535-6. |
| Ru(VI) | 6 | 3,1 | N, OR | | Li, Z-Y, et al. Wuhan University Journal of Natural |
| | | | | | Sciences Vol. 1 No. 1 1996, 89~94 |
| Os(VI) | 6 | 3,1 | N, OR | | Li, Z-Y, et al. Wuhan University Journal of Natural |
| | | | | | Sciences Vol. 1 No. 1 1996, 89~94 |
| Os(VI) | 6 | 2,2 | O, O | | Li, Z-Y, et al. Wuhan University Journal of Natural |
| | | | | | Sciences Vol. 1 No. 1 1996, 89~94 |
| Co(III) | 5, 6 | 1; 1,1 | alkyl, | | Krattinger, B., et al. Bulletin de la Societe Chimique de |
| | | | aryl, | | France, Volume 133, Issue7-8, Pages 721-724 |
| | | | ethynyl | | |

TABLE 2
Exemplary phthalocyanine derivatives that can be modified for use with the present invention

| | Coordination | Bond | Axial | | |
|---|---|---|---|---|---|
| Metal | Number | Order | ligand R | Note | Source |
| Al(III) | 5 | 1 | Cl− | | Porphyrin Handbook, Vol. 3, Chapter 11, Ref. 23, |
| | | | | | 38, 138, 312, 318, 280, 312, 428, 770, 790, 759 |
| Al(III) | 5 | 1 | F−, Br− | | Porphyrin Handbook, Vol. 9, Chapter 42, Ref. 339 |
| Al(III) | 5 | 1 | —OH, —OR | | Porphyrin Handbook, Vol. 3, Chapter 11, Ref. 317, 519, 535, 455, 499; |
| | | | | | Vol. 9, Chapter 42, Ref. 522 |
| Al(III) | 5 | 1 | —OSiEt3 | | Porphyrin Handbook, Vol. 3, Chapter 11, Ref. 680 |
| Al(III) | 5 | 1 | CN− | | Porphyrin Handbook, Vol. 9, Chapter 42, Ref. 316 |
| Al(III) | 5 | 1 | OH | | Porphyrin handbook, Vol 4, Chapter 17, Ref. 42, Vol 7, Chapter 34, Ref. |
| | | | | | 35, 77, 111 |
| Al(III) | 5 | 1 | Cl | | Porphyrin handbook, Vol 4, Chapter 17, Ref. 26, Vol 7, Chapter 34, Ref. |
| | | | | | 18, 33, 35, 53-53, 74, 84-85, 93-96, 104, 108-110, 114 |
| Bi(III) | 5 | 1 | Cl−, Br−, | | Porphyrin Handbook, Vol. 9, Chapter 42, Ref. 398 |
| | | | I−, NO3− | | |
| Co(II) | 5 | 1 | Py | m- | Porphyrin handbook, Vol 2, Chapter 8 Ref. 202, 204 |
| | | | | benziphthalocyanine | |
| Co(III) | 5 | 1 | I−, CN− | | Porphyrin Handbook, Vol. 3, Chapter 11, Ref. 64; Vol. 9, Chapter 42, |
| | | | | | 1225 |
| Co(III) | 6 | 1,1 | CN− | Anionic | Porphyrin Handbook, Vol. 3, Chapter 11, Ref. 118 |
| Co(III) | 6 | 1,1 | 2 Py | m- | Porphyrin handbook, Vol 2, Chapter 8 Ref. 202, 204 |
| | | | | benziphthalocyanine | |
| Cr(III) | 5 | 1 | Cl− | | Porphyrin Handbook, Vol. 3, Chapter 11, Ref. 475, 536, 819 |
| Dy(III) | 5 | 1 | Cl−, Br−, | | Porphyrin Handbook, Vol. 9, Chapter 42, Ref. 508, 681, 600 |
| | | | —OAc, —OCH3 | | |
| Er(III) | 5 | 1 | Cl−, —OAc | | Porphyrin Handbook, Vol. 3, Chapter 11, Ref. 84; Vol. 9, Chapter 42, |
| | | | | | Ref. 513, 1061 |
| Eu(III) | “6” | 1 + | modified | | Porphyrin Handbook, Vol. 9, Chapter 42, Ref. 421 |
| | | dative | acac | | |
| Eu(III) | 5 | 1 | —OAc | | Porphyrin Handbook, Vol. 9, Chapter 42, Ref. 513 |
| Fe(III) | 5 | 1 | SbF6−, | | Porphyrin Handbook, Vol. 9, Chapter 42, Ref. 947 |
| | | | PF6−, | | |
| | | | OCOCF3−, | | |
| | | | ClO4− | | |
| Ga(III) | 5 | 1 | —OH | | Porphyrin Handbook, Vol. 3, Chapter 11, Ref. 151, 475, 536 |
| Ga(III) | 5 | 1 | Cl− | | Porphyrin Handbook, Vol. 3, Chapter 11, Ref. 280, 441, 508 |
| Ga(III) | 5 | 1 | —OSiEt3 | | Porphyrin Handbook, Vol. 3, Chapter 11, Ref. 680 |
| Ga(III) | 5 | 1 | PhCF3, | | Porphyrin Handbook, Vol. 9, Chapter 42, Ref. 329, 328, 326 |
| | | | OPH(4- | | |
| | | | Cl), | | |
| | | | Ph(4- | | |
| | | | CF3) | | |
| Ga(III) | 5 | 1 | Cl | | Porphyrin handbook, Vol 7, Chapter 34, Ref. 33, 67-68, 108-109, 179, |
| | | | | | 184, 188 |
| Ga(III) | 5 | 1 | OAc | | Porphyrin handbook, Vol 7, Chapter 34, Ref. 114 |
| Ga(III) | 5 | 1 | ORO | Bridging | Porphyrin handbook, Vol 7, Chapter 34, Ref. 183 |
| Ga(III) | 5 | 1 | NCRCN | Bridging | Porphyrin handbook, Vol 7, Chapter 34, Ref. 183 |
| Ga(III) | 5 | 1 | CF3R | | Porphyrin handbook, Vol 7, Chapter 34, Ref. 182 |
| Ga(III) | 5 | 1 | O | Bridging | Porphyrin handbook, Vol 7, Chapter 34, Ref. 183 |
| Gd(III) | 5 | 1 | Cl−, Br−, | | Porphyrin Handbook, Vol. 9, Chapter 42, Ref. 508, 1061 |
| | | | —OAc | | |
| Gd(III) | “6” | 1 + | modified | | Porphyrin Handbook, Vol. 9, Chapter 42, Ref. 421 |
| | | dative | acac | | |
| Ge(IV) | 6 | 1,1 | —CCtBu | | Porphyrin Handbook, Vol. 3, Chapter 11, Ref. 62 |
| Ge(IV) | 6 | 1,1 | HCC− | | Porphyrin Handbook, Vol. 3, Chapter 11, Ref. 62 |
| Ge(IV) | 6 | 1,1 | Cl− | | Porphyrin Handbook, Vol. 3, Chapter 11, Ref. 62, 251, 312, 340, 685 |
| Ge(IV) | 6 | 1,1 | —OH | | Porphyrin Handbook, Vol. 3, Chapter 11, Ref. 542, 859 |
| Ge(IV) | 6 | 1,1 | —CC− | 1D | Porphyrin Handbook, Vol. 3, Chapter 11, Ref. 62 |
| | | | | GePor | |
| | | | | wire | |
| | | | | bridged | |
| | | | | by —CC− | |
| Ge(IV) | 6 | 1,1 | —OSiEt3 | | Porphyrin Handbook, Vol. 3, Chapter 11, Ref. 680 |
| Ge(IV) | 6 | 1,1 | 2 OH | | Porphyrin handbook, Vol 7, Chapter 34, Ref. 52, 63, 77, 93 |
| Ge(IV) | 6 | 1,1 | 2 Cl | | Porphyrin handbook, Vol 7, Chapter 34, Ref. 45 |
| Hf(IV) | 6 | 1,1 | Cl−, —OR, | cis Cl−, | Porphyrin Handbook, Vol. 3, Chapter 11, Ref. 280; Vol. 9, Chapter 42, |
| | | | catacholate, | cis —OR | 124, 488, 534 |
| | | | —OH | | |
| Hg(III) | 5 | 1 | Me | | Porphyrin Handbook, Vol. 3, Chapter 11, Ref. 673 |
| Ho(III) | 5 | 1 | Cl−, —OAc | | Porphyrin Handbook, Vol. 3, Chapter 11, Ref. 394, 1061 |
| In(III) | 5 | 1 | Cl− | | Porphyrin Handbook, Vol. 3, Chapter 11, Ref. |
| | | | | | 312, 280, 508, 637, 110, 637, 796, 819 |
| In(III) | 5 | 1 | Br−, I−, | | Porphyrin Handbook, Vol. 9, Chapter 42, Ref. |
| | | | Ph, Bu−, | | 332, 343, 540, 329, 339, 333, 336, 1061 |
| | | | PhR, —OR, | | |
| | | | Me, F−, | | |
| | | | NO2− | | |
| In(III) | 5 | 1 | Cl | | Porphyrin handbook, Vol 7, Chapter 34, Ref. 68-69, 179, 183, 188-189, |
| | | | | | 191 |
| In(III) | 5 | 1 | O | Bridging | Porphyrin handbook, Vol 7, Chapter 34, Ref. 183 |
| In(III) | 5 | 1 | Ph(F4) | Bridging | Porphyrin handbook, Vol 7, Chapter 34, Ref. 183 |
| In(III) | 5 | 1 | NCRCN | Bridging | Porphyrin handbook, Vol 7, Chapter 34, Ref. 183 |
| Ir(III) | 5 | 1 | Cl− | | Porphyrin Handbook, Vol. 3, Chapter 11, Ref. 144 |
| Lu(III) | 5 | 1 | Cl−, —OAc | | Porphyrin Handbook, Vol. 3, Chapter 11, Ref. 394; Vol. 9, Chapter 42, |
| | | | | | Ref. 506, 509, 510, 600 |
| Lu(III) | “6” | 1 + | modified | | Porphyrin Handbook, Vol. 9, Chapter 42, Ref. 421 |
| | | dative | acac | | |
| Mn(III) | 5 | 1 | —OAc | | Porphyrin Handbook, Vol. 3, Chapter 11, Ref. 148, 757 |
| Mn(III) | 5 | 1 | Cl− | | Porphyrin Handbook, Vol. 3, Chapter 11, Ref. 396, 80 |
| Mn(III) | 5 | 1 | —OH | | Porphyrin Handbook, Vol. 3, Chapter 11, Ref. 562, 764, 773, 764 |
| Mo(IV) | 5 | 2 | O | | Porphyrin Handbook, Vol. 3, Chapter 11, Ref. 442 |
| Mo(V) | 5 | 3 | N | | Porphyrin Handbook, Vol. 3, Chapter 11, Ref. 141 |
| Mo(V) | 6 | 1,2 | —OH, O | | Porphyrin Handbook, Vol. 9, Chapter 42, Ref. 154 |
| Nb(IV) | 6 | 1,1 | Cl− | | Porphyrin Handbook, Vol. 9, Chapter 42, Ref. 135 |
| Nb(IV) | 6 | 1,1 | catacholato | | Porphyrin Handbook, Vol. 9, Chapter 42, Ref. 135 |
| Nb(IV) | 6 | 1,1 | SO42− | | Porphyrin Handbook, Vol. 9, Chapter 42, Ref. 135 |
| Nb(IV) | 6 | 1,1 | oxalato | | Porphyrin Handbook, Vol. 9, Chapter 42, Ref. 135 |
| Nb(V) | 7 | 1,1,1 | catacholato, | | Porphyrin Handbook, Vol. 9, Chapter 42, Ref. 135 |
| | | | I− | | |
| Nb(V) | 7 | 1,1,1 | Cl− | | Porphyrin Handbook, Vol. 9, Chapter 42, Ref. 135 |
| Nd(III) | 5 | 1 | —OAc, | | Porphyrin Handbook, Vol. 9, Chapter 42, Ref. 506, 1061 |
| | | | Cl− | | |
| P(V) | 6 | 1,1 | —OH | Cationic | Porphyrin Handbook, Vol. 9, Chapter 42, Ref. 384 |
| Pd(III) | 5 | 1 | Cl− | | Porphyrin Handbook, Vol. 9, Chapter 42, Ref. 246 |
| Pr(III) | 5 | 1 | —OAc | | Porphyrin Handbook, Vol. 9, Chapter 42, Ref. 1061 |
| Re(V) | 5 | 3 | N, | | Porphyrin Handbook, Vol. 3, Chapter 11, Ref. 142, 141; Vol. 9, Chapter |
| | | | NBR3 | | 42, Ref. 157 |
| Re(V) | 6 | 2,1 | O, OEt | | Porphyrin Handbook, Vol. 9, Chapter 42, Ref. 181 |
| Rh(III) | 5 | 1 | Cl− | Can | Porphyrin Handbook, Vol. 3, Chapter 11, Ref. 862, 903 |
| | | | | form | |
| | | | | dative | |
| | | | | bonds | |
| | | | | with | |
| | | | | py, | |
| | | | | DMSO . . . | |
| | | | | etc. | |
| Rh(III) | 5 | 1 | I− | | Porphyrin Handbook, Vol. 9, Chapter 42, Ref. 246 |
| Rh(III) | 6 | 1,1 | CN− | Anionic | Porphyrin Handbook, Vol. 9, Chapter 42, Ref. 505 |
| Ru(III) | 5 | 1 | Cl− | | Porphyrin Handbook, Vol. 9, Chapter 42, Ref. 493 |
| Sb(III) | 5 | 1 | F− | | Porphyrin Handbook, Vol. 9, Chapter 42, Ref. 394 |
| Sb(V) | 6 | 1,1 | —OH, Cl−, | Cationic | Porphyrin Handbook, Vol. 9, Chapter 42, Ref. 396 |
| | | | Br−, I− | | |
| Sb(V) | 6 | 1,1 | 2 Cl | | Porphyrin handbook, Vol 7, Chapter 34, Ref. 198 |
| Sb(V) | 6 | 1,1 | 2 Br | | Porphyrin handbook, Vol 7, Chapter 34, Ref. 198 |
| Sc(III) | 5 | 1 | —OAc | | Porphyrin Handbook, Vol. 9, Chapter 42, Ref. 89 |
| Si(IV) | 6 | 1,1 | —OR | | Porphyrin Handbook, Vol. 1, Chapter 4, Ref. 172-173 |
| Si(IV) | 6 | 1,1 | Cl− | | Porphyrin Handbook, Vol. 3, Chapter 11, Ref. |
| | | | | | 62, 251, 682, 685, 690, 697, 436, 721 |
| Si(IV) | 6 | 1,1 | —OH | | Porphyrin Handbook, Vol. 3, Chapter 11, Ref. 62, 544, 697 |
| Si(IV) | 6 | 1,1 | “R” | | Porphyrin Handbook, Vol. 3, Chapter 11, Ref. 294 |
| Si(IV) | 6 | 1,1 | —CCH, —CCMe, | | Porphyrin Handbook, Vol. 3, Chapter 11, Ref. 62 |
| | | | —CCtBu, —CCPh | | |
| Si(IV) | 6 | 1,1 | OSiR, | | Porphyrin handbook, Vol 4, Chapter 16 Ref. 462 |
| | | | CH3 | | |
| Si(IV) | 6 | 1,1 | OSiR, | | Porphyrin handbook, Vol 4, Chapter 16 Ref. 462, Vol 7, Chapter 34, Ref. |
| | | | OH | | 145 |
| Si(IV) | 6 | 1,1 | 2 OSiR | | Porphyrin handbook, Vol 4, Chapter 16 Ref. 462, Chapter 16 Ref. 462, |
| | | | | | Vol 7, Chapter 34, Ref. 145, 192-193, 195 |
| Si(IV) | 6 | 1,1 | 2 OEt | | Porphyrin handbook, Vol 4, Chapter 16 Ref. 470 |
| Si(IV) | 6 | 1,1 | 2 OPr | | Porphyrin handbook, Vol 4, Chapter 16 Ref. 470 |
| Si(IV) | 6 | 1,1 | 2 | | Porphyrin handbook, Vol 4, Chapter 16 Ref. 470 |
| | | | O(CH2)11CH3 | | |
| Si(IV) | 6 | 1,1 | 2 OR | Various | Porphyrin handbook, Vol 4, Chapter 16 Ref. 472-476, 489, 491, Chapter |
| | | | | | 17, Ref. 13-15, 32-33, Vol 7, Chapter 34, Ref. 44, 127, 192-194 |
| Si(IV) | 6 | 1,1 | 2 Cl | | Porphyrin handbook, Vol 4, Chapter 17, Ref. 32-33, Vol 7, Chapter 34, |
| | | | | | Ref. 44, 68, 127 |
| Si(IV) | 6 | 1,1 | 2 OH | | Porphyrin handbook, Vol 7, Chapter 34, Ref. 44, 52, 63, 77, 93, 127 |
| Si(IV) | 6 | 1,1 | 2 | | Porphyrin handbook, Vol 7, Chapter 34, Ref. 127 |
| | | | O3SCH3 | | |
| Sm(III) | 5 | 1 | —OAc | | Porphyrin Handbook, Vol. 9, Chapter 42, Ref. 600, 1061 |
| Sn(IV) | 6 | 1,1 | Cl−, I− | | Porphyrin Handbook, Vol. 3, Chapter 11, Ref. |
| | | | | | 62, 244, 251, 314, 312, 340, 791, 768 |
| Sn(IV) | 6 | 1,1 | —OH, —OR | | Porphyrin Handbook, Vol. 3, Chapter 11, Ref. 318; Vol. 9, Chapter 42, |
| | | | | | Ref. 437, 359 |
| Sn(IV) | 6 | 1,1 | Cl− | | Porphyrin Handbook, Vol. 3, Chapter 11, Ref. 685, 768, 340 |
| Sn(IV) | 6 | 1,1 | —OSiEt3 | | Porphyrin Handbook, Vol. 3, Chapter 11, Ref. 680 |
| Sn(IV) | 6 | 1,1 | 2 OH | | Porphyrin handbook, Vol 7, Chapter 34, Ref. 52, 63 |
| Sn(IV) | 6 | 1,1 | 2 OR | | Porphyrin handbook, Vol 7, Chapter 34, Ref. 45 |
| Sn(IV) | 6 | 1,1 | 2 Cl | | Porphyrin handbook, Vol 7, Chapter 34, Ref. 45, 154, 186 |
| Ta(IV) | 7 | 1,1,1 | F− | All F- | Porphyrin Handbook, Vol. 9, Chapter 42, Ref. 143 |
| | | | | cis, | |
| | | | | anionic | |
| Ta(V) | 7 | 1,1,1 | Cl− | All Cl- | Porphyrin Handbook, Vol. 9, Chapter 42, Ref. 140, 141 |
| | | | | cis | |
| Ta(V) | 8 | 1,1,1,1 | F− | All F- | Porphyrin Handbook, Vol. 9, Chapter 42, Ref. 143 |
| | | | | cis, | |
| | | | | anionic | |
| Tb(III) | 5 | 1 | —OAc | | Porphyrin Handbook, Vol. 9, Chapter 42, Ref. 600 |
| Tc(V) | 5 | 3 | N | | Porphyrin Handbook, Vol. 9, Chapter 42, Ref. 178 |
| Ti(IV) | “8” | 2 + | modified | all cis | Porphyrin Handbook, Vol. 9, Chapter 42, Ref. 107 |
| | | dative | acac | | |
| Ti(IV) | 6 | 1,1 | catacholato | | Porphyrin Handbook, Vol. 1, Chapter 2, Ref. 234-240; Vol. 9, Chapter 9, |
| | | | | | Ref. 120 |
| Ti(IV) | 5 | 2 | O | | Porphyrin Handbook, Vol. 3, Chapter 11, Ref. |
| | | | | | 140, 341, 386, 390, 395, 562, 769, 774, 736, 757804 |
| Ti(IV) | 6 | 1,1 | Cl− | cis Cl− | Porphyrin Handbook, Vol. 3, Chapter 11, Ref. 759 |
| Ti(IV) | 5 | 2 | O | | Porphyrin handbook, Vol 7, Chapter 34, Ref. 113, 138-139 |
| Tm(III) | 5 | 1 | —OAc | | Porphyrin Handbook, Vol. 9, Chapter 42, Ref. 1061 |
| U(IV) | “8” | 2 + | modified | all cis | Porphyrin Handbook, Vol. 9, Chapter 42, Ref. 107 |
| | | dative | acac | | |
| V(IV) | 5 | 2 | O | | Porphyrin Handbook, Vol. 3, Chapter 11, Ref. |
| | | | | | 79, 38, 43, 70, 39, 138, 140, 129, 151, 152, 293, 99, 346, 384, 380, 429, |
| | | | | | 436, 422, 462, 484, 567, 575, 775, 756 |
| W(IV) | 5 | 2 | O | | Porphyrin Handbook, Vol. 9, Chapter 42, Ref. 156 |
| W(V) | 5 | 3 | N | | Porphyrin Handbook, Vol. 3, Chapter 11, Ref. 141 |
| W(V) | 7 | 1,1,1 | Cl− | | Porphyrin Handbook, Vol. 9, Chapter 42, Ref. 159 |
| W(V) | 6 | 1,2 | —OH, O | | Porphyrin Handbook, Vol. 9, Chapter 42, Ref. 161 |
| W(V) | 5 | 3 | NGeR3, | Cationic | Porphyrin Handbook, Vol. 9, Chapter 42, Ref. 157 |
| | | | NSiR3 | | |
| W(V) | 5 | 3 | NGaX3, | | Porphyrin Handbook, Vol. 9, Chapter 42, Ref. 157 |
| | | | NInX3, | | |
| | | | BX3, | | |
| | | | BR3 | | |
| Y(III) | 5 | 1 | Cl− | | Porphyrin Handbook, Vol. 3, Chapter 11, Ref. 84 |
| Yb(III) | 5 | 1 | —OAc | | Porphyrin Handbook, Vol. 9, Chapter 42, Ref. 600 |
| Zn(III) | 5 | 1 | see page | | Porphyrin Handbook, Vol. 9, Chapter 42, Ref. 487 |
| | | | 122 | | |
| Zr(III) | “8” | 2 + | modified | | Porphyrin Handbook, Vol. 9, Chapter 42, Ref. 126 |
| | | dative | acac | | |
| Zr(IV) | 6 | 1,1 | C7H15COO− | | Porphyrin Handbook, Vol. 9, Chapter 42, Ref. 124 |
| Zr(IV) | 6 | 1,1 | cis Cl−, | | Porphyrin Handbook, Vol. 9, Chapter 42, Ref. 124, 125, 489 |
| | | | cis | | |
| | | | alkoxy, | | |
| | | | catacholato | | |
| Zr(IV) | “8” | 2 + | modified | | Porphyrin Handbook, Vol. 9, Chapter 42, Ref. 491, 492 |
| | | dative | acac | | |
| Zr(IV) | 6 | 1,1 | Cl− | | Porphyrin Handbook, Vol. 3, Chapter 11, Ref. 283, 280, 508 |
| Zr(IV) | 6 | 1,1 | 2 Cl | | Porphyrin handbook, Vol 7, Chapter 34, Ref. 141 |
| Zr(IV) | 6 | 1,1 | 2 OR | Various | Porphyrin handbook, Vol 7, Chapter 34, Ref. 141 |
| | | | | long | |
| | | | | chain | |
| | | | | ligands | |

TABLE 3
Exemplary systems with a metal center bound to an electron donor
(unpaired electrons), capped by an electron acceptor
that can be modified for use with the present invention.

| Het- | | Coor- | | Axial | |
|---|---|---|---|---|---|
| ero- | | dination | Bond | Ligand | |
| cycle | Metal | Number | Order | R | Source |
| Pc | Re(V) | 5 | 3 | N, | Porphyrin Handbook, Vol. 3, |
| | | | | NBR₃ | Chapter 11, Ref. 142, 141; |
| | | | | | Vol. 9, Chapter 42, Ref. 157 |
| Pc | W(V) | 5 | 3 | BX3, | Porphyrin Handbook, Vol. 9, |
| | | | | BR3 | Chapter 42, Ref. 157 |

TABLE 4
Exemplary systems with a metal center bound to an electron donor, capped
by a metal moiety that can be modified for use with the present invention.

| | | Coordination | Bond | | | |
|---|---|---|---|---|---|---|
| Heterocycle | Metal | Number | Order | Axial Ligand R | Note | Source |
| Por | Re(V) | 5 | 3 | N:Cr(III)PorCl | | Tong, C., et al. Inorg. Chem. 1996, 35, 5108-5109 |
| Pc | W(V) | 5 | 3 | NGeR3 | Cationic | Porphyrin Handbook, Vol. 9, Chapter 42, Ref. |
| | | | | | | 157 |
| Pc | W(V) | 5 | 3 | NGaX3, | | Porphyrin Handbook, Vol. 9, Chapter 42, Ref. |
| | | | | NInX3 | | 157 |

TABLE 5
Exemplary systems with a metal center bound to an additional moiety
(metal or non-metal), bridged by one or more atoms (O, S, F . . . etc.) that can be
modified for use with the present invention.

| | | Coordination | Bond | Axial | | |
|---|---|---|---|---|---|---|
| Heterocycle | Metal | Number | Order | Ligand R | Note | Source |
| Por | Al(III) | 5 | 1 | O | Bridging | Porphyrin handbook, Vol 12, Chapter 53, Ref. 266 |
| | | | | | dimer | |
| Por | Fe(III) | 5 | 1 | OTeF₅⁻ | | Porphyrin Handbook, Vol. 6, Chapter 29 Pg. 108 |
| Por | Fe(III) | 5 | 1 | SO₄²⁻ | Dimer | Porphyrin Handbook, Vol. 6, Chapter 29 Pg. 108 |
| Por | Fe(III) | 5 | 1 | HSO₄⁻ | | Porphyrin Handbook, Vol. 6, Chapter 29 Pg. 108 |
| Por | Fe(III) | 5 | 1 | RSO₃⁻ | | Porphyrin Handbook, Vol. 6, Chapter 29 Pg. 108 |
| Por | Fe(III) | 5 | 1 | WS₄²⁻ | | Porphyrin Handbook, Vol. 6, Chapter 29 Pg. 108 |
| Por | Fe(III) | 5 | 1 | SbF₆⁻ | Weakly | Porphyrin Handbook, Vol. 6, Chapter 29 Ref. 400-402 |
| | | | | | coordinating | |
| Por | Fe(III) | 5 | 1 | CF₃CO₂⁻ | Weakly | Porphyrin Handbook, Vol. 6, Chapter 29 Ref. 374, 400 |
| | | | | | coordinating | |
| Por | Fe(III) | 5 | 1 | OTeF3 | | Porphyrin handbook, Vol 7, Chapter 32, Ref. 36, 158-159 |
| Por | Fe(III) | 5 | 1 | ReO4 | | Porphyrin handbook, Vol 7, Chapter 32, Ref. 36, 158-159 |
| Por | Fe(III) | 5 | 1 | BF4 | | Porphyrin handbook, Vol 7, Chapter 32, Ref. 36, 158-159 |
| Por | Fe(III) | 5 | 1 | AsF6 | | Porphyrin handbook, Vol 7, Chapter 32, Ref. 36, 158-159 |
| Por | Fe(III) | 5 | 1 | O | Bridging | Porphyrin handbook, Vol 12, Chapter 53, Ref. 224, 265 |
| | | | | | dimer | |
| Por | Nb(V) | 7 | 1,1,1 | O | NbPor | Watanabe Y., et al. Structure and Bonding (Berlin) (2000), |
| | | | | | dimer with 3 | 97 (Metal-Oxo and Metal-Peroxo Species in Catalytic |
| | | | | | bridging | Oxidations), 61-89. |
| | | | | | oxos | |
| Por | Fe(III) | 5 | 1 | O | mu-oxo | Suzuki, M., et al. Chemistry Letters Vol. 32, No. 7 (2003) |
| | | | | | dimer | |
| Por | Fe(III) | 5 | 1 | O, OH | mu-oxo and | Nanthakumar, A., et al. J. Am. Chem. SOC. 1993, 115, |
| | | | | | mu-hydroxy | 8513-8514 8513 |
| | | | | | bridged with | |
| | | | | | Cu(II); | |
| | | | | | cationic | |
| Por | Zr(IV) | 7 | ? | OH | ZrPor dimer | Kim, H-J, et al. Chemistry Letters Vol. 22 (1993), No. 5 |
| | | | | | bridged by 3 | p. 807 |
| | | | | | OH−; | |
| | | | | | cationic | |
| Por | Zr(IV) | 7 | 1,1,dative | O, OH | ZrPor dimer | Kim, H-J, et al. Chemistry Letters Vol. 22 (1993), No. 5 |
| | | | | | bridged by 2 | p. 807 |
| | | | | | OH− and | |
| | | | | | O²⁻ | |
| Por | Sc(III) | 5 | 1 | O | oxo-bridged | Leenstra, W. R., et al. Journal of Chemical Physics (1979), |
| | | | | | dimer | 71(8), 3535-6. |
| Pc | Fe(III) | 5 | 1 | SbF₆⁻, | | Porphyrin |
| | | | | PF₆⁻, OCOCF₃⁻, ClO₄⁻ | | Handbook, Vol. 9, |
| | | | | | | Chapter 42, Ref. |
| | | | | | | 947 |
| Pc | Ga(III) | 5 | 1 | ORO | Bridging | Porphyrin handbook, Vol 7, Chapter 34, Ref. 183 |
| Pc | Ga(III) | 5 | 1 | NCRCN | Bridging | Porphyrin handbook, Vol 7, Chapter 34, Ref. 183 |
| Pc | Ga(III) | 5 | 1 | O | Bridging | Porphyrin handbook, Vol 7, Chapter 34, Ref. 183 |
| Pc | In(III) | 5 | 1 | Ph(F4) | Bridging | Porphyrin handbook, Vol 7, Chapter 34, Ref. 183 |
| Pc | In(III) | 5 | 1 | NCRCN | Bridging | Porphyrin handbook, Vol 7, Chapter 34, Ref. 183 |
| Pc | Nb(IV) | 6 | 1,1 | SO₄²⁻ | | Porphyrin Handbook, Vol. 9, Chapter 42, Ref. 135 |

TABLE 6
Exemplary systems with a metal bound to organic fragments (R), which are
used to bind to an additional organic structure that can be modified for use with the
present invention.

| | | Coordination | Bond | | | |
|---|---|---|---|---|---|---|
| Heterocycle | Metal | Number | Order | Ligand R | Note | Source |
| Por | Dy(III) | “6” | 1 + dative | acac | | Porphyrin handbook, Vol 12, Chapter 53, Ref. 267 |
| Por | Er(III) | “6” | 1 + | acac | | Porphyrin handbook, Vol 12, Chapter 53, Ref. 269 |
| | | | dative | | | |
| Por | Fe(III) | 5 | 1 | RC₂⁻ | Acetylide | Porphyrin Handbook, Vol. 6, Chapter 29 Pg. 108 |
| | | | | | compound | |
| Por | Ga(III) | 5 | 1 | alkyl, | | Kadish, K. M., et al. Inorg. Chem. 1985, 24, 4521-4528 |
| | | | | alkenyl, | | |
| | | | | CCPh | | |
| Por | Ga(III) | 5 | 1 | alkyl, | | Kadish, K. M., et al. Inorg. Chem. 1989, 28, 2518-2523 |
| | | | | alkenyl, | | |
| | | | | CCPh | | |
| Por | Gd(III) | “6” | 1 + | acac | | Porphyrin handbook, Vol 12, Chapter 53, Ref. 265 |
| | | | dative | | | |
| Por | Ho(III) | “6” | 1 + | acac | | Porphyrin handbook, Vol 12, Chapter 53, Ref. 268 |
| | | | dative | | | |
| Por | In(III) | 5 | 1 | alkyl, | | Kadish, K. M., et al. Inorg. Chem. 1989, 28, 2518-2523 |
| | | | | alkenyl, | | |
| | | | | CCPh | | |
| Por | In(III) | “6” | 1 + | acac | | Porphyrin handbook, Vol 12, Chapter 53, Ref. 264 |
| | | | dative | | | |
| Por | Tb(III) | “6” | 1 + | acac | | Porphyrin handbook, Vol 12, Chapter 53, Ref. 266 |
| | | | dative | | | |
| Por | Mo(IV) | 5 | 2 | NR | | Berraeu, L. M., et al. Inorg. Chem. 2005, 44, 7304-7306 |
| Por | Ti(IV) | 5 | 2 | NR | Intermetal | Berraeu, L. M., et al. Inorg. Chem. 2005, 44, 7304-7306 |
| | | | | | imido | |
| | | | | | transfer | |
| | | | | | from Mo | |
| | | | | | to Ti | |
| Por | Ti(IV) | 6 | 1,1 | catacholate | reaction | Du, G., et al. Organometallics 2004, 23, 4230-4235 |
| | | | | | of Ti(II) | |
| | | | | | complex | |
| | | | | | with | |
| | | | | | aldehydes | |
| Por | Ti(IV) | 5 | 2 | NNR2, NR | highly | Thorman, J. L., et al. Inorg. Chem. 2000, 39, 1301-1304 |
| | | | | | reactive | |
| | | | | | with | |
| | | | | | oxygenated | |
| | | | | | species | |
| Por | Zr(IV) | 5 | 2 | NR | | Thorman, J. L., et al. Inorg. Chem. 1999, 38, 3814-3824 |
| Por | Hf(IV) | 5 | 2 | NR | | Thorman, J. L., et al. Inorg. Chem. 1999, 38, 3814-3824 |
| Por | Zr(IV) | 6 | 1,1 | N,O | Bidentate | Thorman, J. L., et al. Inorg. Chem. 1999, 38, 3814-3824 |
| Por | Zr(IV) | 6 | 1,1 | N,O | Bidentate | Thorman, J. L., et al. Inorg. Chem. 1999, 38, 3814-3824 |
| Por | Os(II) | 5 | 2 | CRR′ | | Woo, L. K., et al. Organometallics 1992, 11, 2344-2346 |
| Por | Os(II) | 5 | 2 | SiRR′ | | Woo, L. K., et al. Organometallics 1991, 10, 3977-3982 |
| Por | Ru(IV) | 5 | 2 | CRR′ | air and | Che, C-M, et al. J. Am. Chem. Soc. 2001, 123, 4119-4129 |
| Por | Fe(II) | 5 | 2 | CRR′, | light | Ziegler, C. J., et al. Journal of Organometallic |
| | | | | CCRR′ | sensitive | Chemistry 528 (1997) 83-90 |
| Por | Mn(V) | 5 | 2 | CRR′ | | Jones, T. K., et al. Inorganica Chimica Acfa, 206 |
| | | | | | | (1993) 5-7 |
| Por | Ru(II) | 5 | 2 | CRR′ | air-stable | Wada, S., et al. Acta Cryst. (2003). C59, m369-m370 |
| Por | Co(III) | 5, 6 | 1; 1,1 | alkyl, aryl, | | Krattinger, B., et al. Bulletin de la Societe Chimique |
| | | | | ethynyl | | de France, Volume 133, Issue7-8, Pages 721-724 |
| Pc | Eu(III) | “6” | 1 + | modified | | Porphyrin Handbook, Vol. 9, Chapter 42, Ref. 421 |
| | | | dative | acac | | |
| Pc | Ga(III) | 5 | 1 | PhCF3, | | Porphyrin Handbook, Vol. 9, Chapter 42, Ref. |
| | | | | OPH(4-Cl), | | 329, 328, 326 |
| | | | | Ph(4-CF3) | | |
| Pc | Hf(IV) | 6 | 1,1 | Cl−, —OR, | cis Cl−, cis | Porphyrin Handbook, Vol. 3, Chapter 11, Ref. 280; |
| | | | | catacholate, | —OR | Vol. 9, Chapter 42, 124, 488, 534 |
| | | | | —OH | | |
| Pc | In(III) | 5 | 1 | Br−, I−, Ph, | | Porphyrin Handbook, Vol. 9, Chapter 42, Ref. |
| | | | | Bu−, PhR, | | 332, 343, 540, 329, 339, 333, 336, 1061 |
| | | | | —OR, Me, | | |
| | | | | F−, NO₂ | | |
| Pc | Lu(III) | “6” | 1 + | modified | | Porphyrin Handbook, Vol. 9, Chapter 42, Ref. 421 |
| | | | dative | acac | | |
| Pc | Nb(IV) | 6 | 1,1 | catacholato | | Porphyrin Handbook, Vol. 9, Chapter 42, Ref. 135 |
| Pc | Nb(IV) | 6 | 1,1 | oxalato | | Porphyrin Handbook, Vol. 9, Chapter 42, Ref. 135 |
| | | | | catacholato, | | |
| Pc | Nb(V) | 7 | 1,1,1 | I- | | Porphyrin Handbook, Vol. 9, Chapter 42, Ref. 135 |
| Pc | Ti(IV) | “8” | 2 + | modified | all cis | Porphyrin Handbook, Vol. 9, Chapter 42, Ref. 107 |
| | | | dative | acac | | |
| Pc | Ti(IV) | 6 | 1,1 | catacholato | | Porphyrin Handbook, Vol. 1, Chapter 2, Ref. 234-240; |
| | | | | | | Vol. 9, Chapter 9, Ref. 120 |
| Pc | U(IV) | “8” | 2 + | modified | all cis | Porphyrin Handbook, Vol. 9, Chapter 42, Ref. 107 |
| | | | dative | acac | | |
| Pc | W(V) | 5 | 3 | NSiR3 | Cationic | Porphyrin Handbook, Vol. 9, Chapter 42, Ref. 157 |
| | | | | see page | | |
| Pc | Zn(III) | 5 | 1 | 122 | | Porphyrin Handbook, Vol. 9, Chapter 42, Ref. 487 |
| Pc | Zr(III) | “8” | 2 + | modified | | Porphyrin Handbook, Vol. 9, Chapter 42, Ref. 126 |
| | | | dative | acac | | |
| Pc | Zr(IV) | 6 | 1,1 | C₇H₁₅COO⁻ | | Porphyrin Handbook, Vol. 9, Chapter 42, Ref. 124 |
| | | | | cis Cl−, cis | | |
| Pc | Zr(IV) | 6 | 1,1 | alkoxy, | | Porphyrin Handbook, Vol. 9, Chapter 42, Ref. |
| | | | | catacholato | | 124, 125, 489 |
| Pc | Zr(IV) | “8” | 2 + | modified | | Porphyrin Handbook, Vol. 9, Chapter 42, Ref. 491, 492 |
| | | | dative | acac | | |
| Pc | Gd(III) | “6” | 1 + | modified | | Porphyrin Handbook, Vol. 9, Chapter 42, Ref. 421 |
| | | | dative | acac | | |

TABLE 7
Exemplary systems with a metal center bound to an electron donor,
uncapped, that can be modified for use with the present invention.

| Hetero | | Coordination | Bond | | |
|---|---|---|---|---|---|
| cycle | Metal | Number | Order | Axial R | Source |
| Por | Fe(IV) | 5 | 2 | O | Porphyrin Handbook, Vol. 6, Chapter 29 Pg. 110 |
| Por | Fe(IV) | 5 | 2 | O | Porphynn handbook, Vol 7, Chapter 32, Ref. 231, 253, 258-272 |
| Por | Mn(IV) | 5 | 2 | O | Porphyrin Handbook, Vol. 6, Chapter 29 Ref. 229 |
| Por | Sb(V) | 5 | 2 | O | Porphyrin handbook, Vol 11, Chapter 50, Ref. 22 |
| Por | Mo(IV) | 5 | 2 | S, | Berraeu, L. M.,et al. Inorg. Chem. 1995, 34, 3485-3490 |
| | | | | Se | |
| Por | Mn(V) | 5 | 3 | N | Woo, L. K., et al. J. Am. Chem. SOC. 1991, 113, 8478-8484 |
| Por | Cr(V) | 5 | 3 | N | Campochiaro, C., et al. Inorg. Chem. 1985, 24, 449-450 449 |
| Pc | Tc(V) | 5 | 3 | N | Porphyrin Handbook, Vol. 9, Chapter 42, Ref. 178 |
| Pc | Ti(IV) | 5 | 2 | O | Porphyrin Handbook, Vol. 3, Chapter 11, Ref. |
| | | | | | 140, 341, 386, 390, 395, 562, 769, 774, 736, 757804 |
| Pc | Ti(IV) | 5 | 2 | O | Porphyrin handbook, Vol. 7, Chapter 34, Ref. 113, 138-139 |
| Pc | V(IV) | 5 | 2 | O | Porphyrin Handbook, Vol. 3, Chapter 11, Ref. |
| | | | | | 79, 38, 43, 70, 39, 138, 140, 129, 151, 152, 293, 99, 346, 384, 380, 429, |
| | | | | | 436, 422, 462, 484, 567, 575, 775, 756 |
| Pc | W(IV) | 5 | 2 | O | Porphyrin Handbook, Vol. 9, Chapter 42, Ref. 156 |
| Pc | W(V) | 5 | 3 | N | Porphyrin Handbook, Vol. 3, Chapter 11, Ref. 141 |

TABLE 8
Exemplary ligands for use with the invention that can be modified for use with the present invention.

| Ligand Type | Ligand | Note | Ligand Preparation/Coordination | Modification/R Group Incorporation | Useful References |
|---|---|---|---|---|---|
| Carbene | CRR′ | Discher, | Preparation from ketenes or diazo (from | 1) Fischer-type metal- | 1) Carbene Chemistry: From |
| | | Shrock, | tosylhydrazone) derivatives (removal of | carbene complexes (see | Fleeting Intermediates to Powerful |
| | | Carbenoid | CO/N2 with light and/or heating); | Chapter 8 of Carbene | Reagents, edited by Guy Bertrand; |
| Carbene | CNR | | tosylhydrazone salts react with metals to | Chemistry, edited by Guy | 2) The Chemistry of diazonium and |
| Carbene | CCRR′ | vinylidene | form metal carbenes (used in | Bertrand); 2) Diazo | diazo groups, Part 2, by Saul Patai |
| Carbene | heterocyclic | | cyclopropanation and epoxidation | derivatives of desired R | |
| | carbenes | | reactions). | Groups (preparation | |
| | | | | procedures in The | |
| | | | | Chemistry of Diazonium | |
| | | | | and Diazo Groups, Part 2) | |
| Hydrocarbyl | —CR3 | | Preparation through reaction with | Numerous. | |
| Hydrocarbyl | —Ph | | organolithium reagent/halide | | |
| Hydrocarbyl | —CHCR2 | | derivative/triflate derivative. | | |
| Hydrocarbyl | —CCR | | | | |
| Metal-Anionic | —OCR3 | | 1) metathesis between metal halides and | Modified acac: soft | |
| Oxygen | | | alikal metal salts; 2) direct reaction of | enolate formation and | |
| Metal-Anionic | —OSiR3 | | the alcohols with metal halides in the | acylation on treatment | |
| Oxygen | | | presence of a base; 3) protonolysis of | with MgBr2.OEt2, i- | |
| | | | metal alkyls, amides or alkoxides with | Pr2NEt, and various | |
| | | | more acidic alcohols | acylating agents, C- | |
| Metal-Anionic | —OR | | | acylation of 1,3- | |
| Oxygen | | | | dicarbonyl compounds | |
| Metal-Anionic | acac | | | and malononitrile with | |
| Oxygen | | | | acid chlorides . . . etc.; | |
| Metal-Anionic | oxalate | | | Modified catecholates: | |
| Oxygen | | | | aromatic substitution | |
| Metal-Anionic | catecholate | | | reactions, conjugate | |
| Oxygen | | | | addition of vinyl-, aryl-, | |
| Metal-Anionic | hydroxamate | | | and heteroarylcopper | |
| Oxygen | | | | reagents to | |
| Metal-Anionic | RCOO- | | | cyclobutenediones | |
| Oxygen | | | | followed by thermal | |
| | | | | rearrangement . . . etc. | |
| | | | | (Many catachol | |
| | | | | derivatives exist) | |
| Metal-Boryl | BR3 | | BR3: Hydroboration with alkenes or | See Ligand Preparation. | 1) The Organic Chemistry of Boron, |
| Metal-Boryl | —BR2 (boryl) | | alkynes, protonolysis (under mild | | by William Gerrard; |
| Metal-Boryl | BR (borylene) | BO = 2 | conditions with carboxylic acids), | | 2) Preparation of Organoboranes: |
| | | | transmetallation, haloboronation. | | Reagents for Organic Synthesis, |
| | | | Friedel-Crafts type haloboronation, | | by K. Smith (Review) |
| | | | reaction of boric acid with an alcohol to | | |
| | | | form boric acid esters . . . etc.; BR2: | | |
| | | | haloboryl complex; BR: highly reactive | | |
| | | | (introduced as dihaloboryl complex) | | |
| Metal-Nitrogen | —NR2 | | Imido (NR): Reaction with metal oxo | Numerous. | 1) Nitrido and imido transition metal |
| Covalent | | | complexes with primary amines, | | complexes of Groups 6-8, |
| Metal-Nitrogen | NR | BO = 2 | decomposition of azides, deprotonation | | by Rebecca A. Eikey, et al. (Review); |
| Covalent | | | of coordinated amines, alkylation or | | 2) Methods of synthesis of Group 4-9 |
| Metal-Nitrogen | azolyl | Multiple binding modes | acylation of nitrido ligand . . . etc.; Amido | | transition metal imido complexes, |
| Covalent | | | (NR2): treating a halide complex with an | | by D. N. Zarubin and N. A. Ustynyuk |
| | | | alkali amide, treating an alkoxide | | (Review). |
| Metal-Nitrogen | bis-sulfonamido | | complex with an amine, deprotonation of | | |
| Covalent | | | a coordinated amine, oxidative addition | | |
| Metal-Nitrogen | pyrazolylborate | | of an amine | | |
| Covalent | | | | | |
| Metal-Nitrogen | diketinminate | | | | |
| Covalent | | | | | |
| Metal-Phosphido | PR2 | BO = 1 or 2 | Phosphenium (PR2): halogen abstraction | | The chemistry of transition metal |
| | | | by AlCl3 or PF6 from precursor | | complexes containing a phosphenium |
| | | | halophosphine complex (PR2X), hydride | | ligand, by Hiroshi Nakazawa (Review). |
| | | | abstraction by BR3 from precursor | | |
| | | | phosphine complex (PR2H), protic | | |
| | | | attack toward a phosphite complex | | |
| Metal-thiolate | —SR | | Refer to Metal-Anionic Oxygen section. | Numerous. | |
| Neutral | OR2 | | Dative. | Numerous. | |
| Chalcogen Donor | | | | | |
| Neutral | SR2, SR2O | | | | |
| Chalcogen Donor | | | | | |
| Nitrogenous Base | NR3 | | | | |
| Nitrogenous Base | py, bipy, terpy, | | Dative. | Numerous. | |
| | bisiminylpyridine . . . | | | | |
| | etc. | | | | |
| Nitrogenous Base | diamines, | | | | |
| | polyamines | | | | |
| Nitrogenous Base | imines | | | | |
| Nitrogenous Base | oxazolines | | | | |
| Nitrogenous Base | bisimines | | | | |
| Nitrogenous Base | bisoxazolines | | | | |
| Nitrogenous Base | N2 | bridging ligand | | | |
| Nitrogenous Base | en, to | | | | |
| Phosphine | PR3 | | Dative. | Numerous. | |
| Phosphine | bisphosphines | Examples on Pg. 34 (Hartwig) | | | |
| Pi Ligands | Cp | | Deprotonation of corresponding | Numerous. | |
| Pi Ligands | Allyl | | hydrocarbon with a base. | | |
| Pi Ligands | Benzyl | | | | |
| Pi Ligands | trimethylenemethane | | | | |

TABLE 9
Exemplary heterocycle peripheral substituents for use with the invention
that can be modified for use with the present invention.

| | | Number | | | | |
|---|---|---|---|---|---|---|
| | | of sub- | | | | |
| Substituent | Complex | stituents | Position | Synthesis | Notes | Reference |
| CCH | Ni(OEP) | 1 | meso | Eglinton | | Porphyrin Handbook, Vol. 1, Chapter 1, Ref. 53 |
| | | | | reaction: | | |
| | | | | ethylene | | |
| | | | | bromide | | |
| | | | | substituted | | |
| | | | | porphyrin | | |
| | | | | treated with | | |
| | | | | NaH to | | |
| | | | | give | | |
| | | | | alkyne. | | |
| | | | | Dimer | | |
| | | | | formed in | | |
| | | | | py solution | | |
| | | | | with | | |
| | | | | Cu(Oac)2 | | |
| CC(por) | Zn(Por) | 1 or 2 | meso | | | Porphyrin Handbook, Vol. 1, Chapter 1, Ref. 54 |
| CCX, | Zn(Por) | 1 to 4 | meso | Protecting | | Porphyrin Handbook, Vol. 1, Chapter 1, Ref. 86-92 |
| X = various | derivatives | | | group such | | |
| Ar | | | | as TIPS and | | |
| | | | | TMS | | |
| | | | | replaced | | |
| Br | various | various | meso, | Free base | | Porphyrin Handbook, Vol. 1, Chapter 1, Ref. 57-58, |
| | | | beta | porphyrin | | Vol. 2, Chapter 9, Ref. 55-63 |
| | | | | treated with | | |
| | | | | NBS. | | |
| | | | | Selective | | |
| | | | | bromination | | |
| | | | | of free | | |
| | | | | meso | | |
| | | | | positions in | | |
| | | | | presence of | | |
| | | | | variety of | | |
| | | | | substituents | | |
| | | | | possible | | |
| CCAr | Zn and | 1 to 4 | meso | | | Porphyrin Handbook, Vol. 1, Chapter 1, Ref. 78-79 |
| bridges | Au por | | | | | |
| (varying | derivatives | | | | | |
| lengths) | | | | | | |
| ArCCAr | Zn(por) | 1 to 4 | meso | Copper-free | | Porphyrin Handbook, Vol. 1, Chapter 1, Ref. 84-85 |
| linkers | | | | Sonogashir | | |
| | | | | a coupling | | |
| Ar | various | 1 to 4 | meso | | Various: | Porphyrin Handbook, Vol. 1, Chapter 1, Ref. 100-105, |
| derivatives | | | | | Mes, | 165, 187, 189, 191, 229, Vol. 3, Chapter 15, |
| | | | | | pentafluorophenyl, | Ref. 57 |
| | | | | | 3,5-di t- | |
| | | | | | Bu Ph, | |
| | | | | | pTol etc. | |
| OTf | Zn(10,15, | 2 | beta | Free base | | Porphyrin Handbook, Vol. 1, Chapter 1, Ref. 111-115 |
| | 20-Ar3, | | | biborylporphrin | | |
| | 5-OTf | | | treated | | |
| | por) | | | with oxone, | | |
| | | | | PhNTf2, | | |
| | | | | Zn(OAc)2 | | |
| CCTMS | Zn(10,15, | 2 | beta | OTf | | Porphyrin Handbook, Vol. 1, Chapter 1, Ref. 111-115 |
| | 20-Ar3, | | | complex | | |
| | 5- | | | treated with | | |
| | CCTMS | | | TMS-CCH. | | |
| | por) | | | Bridged | | |
| | | | | dimer | | |
| | | | | formed | | |
| | | | | upon | | |
| | | | | treatment | | |
| | | | | with | | |
| | | | | K2CO3 and | | |
| | | | | Cu(OAc)2 | | |
| 4-hydroxy | Zn or | 1 | meso | | | Porphyrin Handbook, Vol. 1, Chapter 1, Ref. 165 |
| phenyl | Pt(10,15, | | | | | |
| | 20-Ar3, | | | | | |
| | 5- | | | | | |
| | hydroxy | | | | | |
| | phenyl | | | | | |
| | por) | | | | | |
| pyrimidine | Zn, Pt, | 1 to 2 | meso | | | Porphyrin Handbook, Vol. 1, Chapter 1, Ref. 165, |
| derivatives | or free | | | | | 329-330 |
| | base(10, | | | | | |
| | 15,20- | | | | | |
| | Ar3,5- | | | | | |
| | pyrimidine | | | | | |
| | por) | | | | | |
| OBn | Zn(10,15, | 1 | meso | | | Porphyrin Handbook, Vol. 1, Chapter 1, Ref. 165 |
| | 20-Ar3, | | | | | |
| | 5-OBn | | | | | |
| | por) | | | | | |
| 4-alkoxyphenyl | Zn or | 1 | meso | Williamson | | Porphyrin Handbook, Vol. 1, Chapter 1, Ref. 167-168 |
| | free | | | ether | | |
| | base(10, | | | synthesis | | |
| | 15,20- | | | | | |
| | Ar3,5- | | | | | |
| | alkoxyphenyl | | | | | |
| | por) | | | | | |
| 3,5- | Zn(10,15, | 1 | meso | | | Porphyrin Handbook, Vol. 1, Chapter 1, Ref. 185 |
| dihydroxy- | 20-Ph3, | | | | | |
| phenyl | 5- | | | | | |
| | dihydroxy- | | | | | |
| | phenyl | | | | | |
| | por) | | | | | |
| 3,5- | Zn(10,15, | 1 | meso | dihydroxy | | Porphyrin Handbook, Vol. 1, Chapter 1, Ref. 185 |
| dialkoxyphenyl | 20-Ph3, | | | phenyl used | | |
| | 5- | | | as | | |
| | dialkoxy | | | precursor | | |
| | phenyl | | | | | |
| | por) | | | | | |
| NO2 | free base | 1 | beta | | | Porphyrin Handbook, Vol. 1, Chapter 1, Ref. 187, |
| | 5,10,15, | | | | | 205, 370 |
| | 20-Ar4 | | | | | |
| | por, | | | | | |
| | Cu(TPP), | | | | | |
| | Ni(5,10, | | | | | |
| | 15,20- | | | | | |
| | Ar4, | | | | | |
| | NO2 | | | | | |
| | por) | | | | | |
| amido | Ni(TPP) | 1 | beta | | | Porphyrin Handbook, Vol. 1, Chapter 1, Ref. 188 |
| CHO | Zn(10,20- | 2 | 5,15 | | | Porphyrin Handbook, Vol. 1, Chapter 3, Ref. 155-156 |
| | R2, | | meso | | | |
| | 5,15- | | | | | |
| | CHO | | | | | |
| | por) | | | | | |
| CHO | free base | 1 | meso | | | Porphyrin Handbook, Vol. 1, Chapter 1, Ref. 228-229 |
| | (10,20- | | | | | |
| | Ar2,5- | | | | | |
| | CHO | | | | | |
| | por), | | | | | |
| | Ni(10,15, | | | | | |
| | 20-Ar3, | | | | | |
| | 5-CHO | | | | | |
| | por) | | | | | |
| CH(pyrrole)2 | Ni(Ar)3(por) | 1 | meso | | | Porphyrin Handbook, Vol. 1, Chapter 1, Ref. 229 |
| OH | free | 2 | 3,7 beta | | | Porphyrin Handbook, Vol. 1, Chapter 1, Ref. 260 |
| | base(10, | | | | | |
| | 15,20- | | | | | |
| | Ar3,5- | | | | | |
| | OH por) | | | | | |
| C(CN)2 | Zn(10,20- | 2 | 5,15 | | | Porphyrin Handbook, Vol. 1, Chapter 1, Ref. 266-267 |
| | Ar2, | | meso | | | |
| | 5,15- | | | | | |
| | (C(CN)2)2 | | | | | |
| | por) | | | | | |
| imidazole | Zn(10, | 1 | meso | | various R′ | Porphyrin Handbook, Vol. 1, Chapter 1, Ref. 288-302 |
| | 20- | | | | used as | |
| | R2)(15- | | | | linkers | |
| | R′)(por) | | | | | |
| pyridyl | Co(5,10- | 2 | 5,15 | | | Porphyrin Handbook, Vol. 1, Chapter 1, Ref. 306-307 |
| | pyridal2 | | meso | | | |
| | por) | | | | | |
| 4-py | Zn(5,15- | 2 | 5,15 | | py on zinc | Porphyrin Handbook, Vol. 1, Chapter 1, Ref. 315-316 |
| | py2 por), | | meso | | complex | |
| | Mn(5,15- | | | | coordinated | |
| | py2 | | | | to | |
| | por)Cl | | | | tricarbonylrhenium(I) | |
| | | | | | to form | |
| | | | | | porphyrin | |
| | | | | | squares | |
| 4-py | free base | 2 | 5,10 | | 2 + 2 | Porphyrin Handbook, Vol. 1, Chapter 1, Ref. 324 |
| | 5,10-py2 | | meso | | metallacycles | |
| | por | | | | formed by | |
| | | | | | coordinating | |
| | | | | | to | |
| | | | | | RuCl2(DMSO)2(CO) | |
| 4-py | free base | 1 | meso | | | Porphyrin Handbook, Vol. 1, Chapter 1, Ref. 355-356 |
| | 10,15,20- | | | | | |
| | Ar3 5- | | | | | |
| | py | | | | | |
| pyrazole | Zn(10- | 1 | meso | | self- | Porphyrin Handbook, Vol. 1, Chapter 1, Ref. 331 |
| derivatives | Ar, 15- | | | | assembled | |
| | Ar′,5- | | | | trimer via | |
| | pyrazole | | | | H-bond | |
| | por) | | | | interactions | |
| | | | | | between | |
| | | | | | ester | |
| | | | | | carbonyl | |
| | | | | | on 15-aryl | |
| | | | | | group and | |
| | | | | | pyrazole | |
| | | | | | nitrogen | |
| | | | | | atom | |
| 4-pyridal | Zn(15- | 1 | meso | | | Porphyrin Handbook, Vol. 1, Chapter 1, Ref. 350 |
| phenyl | Ar,5- | | | | | |
| derivatives | pyridal | | | | | |
| | phenyl | | | | | |
| | por) | | | | | |
| imide | Zn(10,20- | 1 | meso | | | Porphyrin Handbook, Vol. 1, Chapter 1, Ref. 351 |
| derivatives | Ar2,5- | | | | | |
| | imide | | | | | |
| | por) | | | | | |
| Pt(R2 R′), | Ni(10,20- | 1 | meso | | R = PPh3, | Porphyrin Handbook, Vol. 1, Chapter 1, Ref. 353-354 |
| Pd(R2 R′) | Ar2,5- | | | | R′ = Br | |
| | M por) | | | | | |
| Pt(R2 R′) | Ni(10,20- | 1 | meso | | R = PPh3, | Porphyrin Handbook, Vol. 1, Chapter 1, Ref. 355-356 |
| | Ar2,15- | | | | R′ = OTf | |
| | Ph,5-Pt | | | | | |
| | por) | | | | | |
| NH2 | Ni(10,15, | 1 | meso | | | Porphyrin Handbook, Vol. 1, Chapter 1, Ref. 357 |
| | 20-Ph3, | | | | | |
| | 5-NH2 | | | | | |
| | por) | | | | | |
| NH2 | Ni(5,10, | 1 | beta | | | Porphyrin Handbook, Vol. 1, Chapter 1, Ref. 370 |
| | 15,20 | | | | | |
| | Ar4,2- | | | | | |
| | NO2,3- | | | | | |
| | NH2) | | | | | |
| Pt acetylide | Ni(10,15, | 1 to 4 | meso | | | Porphyrin Handbook, Vol. 1, Chapter 1, Ref. 360-361 |
| bridges | 20-Ar3, | | | | | |
| | 5-Pt | | | | | |
| | bridge | | | | | |
| | por). | | | | | |
| | Zn(5,10, | | | | | |
| | 15,20-Pt | | | | | |
| | bridge | | | | | |
| | por) | | | | | |
| 4-py | various | 1 | meso | | various | Porphyrin Handbook, Vol. 1, Chapter 1, Ref. 365-366 |
| derivatives | | | | | | |
| MeOH | Zn(OEP) | 1 | meso | | | Porphyrin Handbook, Vol. 1, Chapter 3, Ref. 134-136 |
| | dervivatives | | | | | |
| MeOH | Zn(OEP) | 1 | beta | | | Porphyrin Handbook, Vol. 1, Chapter 3, Ref. 169 |
| | dervivatives | | | | | |
| ROH | various | various | meso, | | | Porphyrin Handbook, Vol. 1, Chapter 3, Ref. 143, |
| | | | beta | | | 145, 169 |
| CN | Zn(10,20- | 2 | 5,15 | | | Porphyrin Handbook, Vol. 1, Chapter 3, Ref. 155-156 |
| | R2, | | meso | | | |
| | 5,15-CN | | | | | |
| | por) | | | | | |
| COCH3 | various | 1 or 2 | beta, 2,8 | | | Porphyrin Handbook, Vol. 1, Chapter 3, Ref. 155-156, |
| | | | and 2,12 | | | 169 |
| | | | beta | | | |
| vinyl | OEP | 1 | beta | | | Porphyrin Handbook, Vol. 1, Chapter 3, Ref. 169 |
| | derivatives | | | | | |
| OR | TPP | 1 | beta | | | Porphyrin Handbook, Vol. 1, Chapter 9, Ref. 65 |
| SR | TPP | 1 | beta | | | Porphyrin Handbook, Vol. 1, Chapter 9, Ref. 65 |
| NHR | TPP | 1 | beta | | | Porphyrin Handbook, Vol. 1, Chapter 9, Ref. 188 |
| NRR′ | TPP | 1 | beta | | | Porphyrin Handbook, Vol. 1, Chapter 9, Ref. 65 |
| 1,2,3- | various | 1 or 4 | meso | Click | | Porphyrin Handbook, Vol. 1, Chapter 9, Ref. 300-301 |
| triazole | meso | | | reaction | | |
| | substituted | | | | | |
| | tri- | | | | | |
| | aryl por | | | | | |
| 4-azide Ar | various | various | meso | | | Porphyrin Handbook, Vol. 1, Chapter 9, Ref. 297-302 |
| derivatives | | | | | | |
| POPh2 | various | 1 or 2 | meso, | | | Porphyrin Handbook, Vol. 3, Chapter 13, Ref. 76 |
| | | | 5,15- | | | |
| | | | meso | | | |
| I | Zn(10,15, | 1 | meso | | | Porphyrin Handbook, Vol. 3, Chapter 13, Ref. 183 |
| | 20-Ar3, | | | | | |
| | 5-I por) | | | | | |
| 4- | 5,15- | 2 | meso | | | Porphyrin Handbook, Vol. 3, Chapter 15, Ref. 106 |
| carboxyphenyl | hydroxy | | | | | |
| | phenyl, | | | | | |
| | 10,20- | | | | | |
| | carboxy- | | | | | |
| | phenyl | | | | | |
| | por | | | | | |
| RCOOH | various | 2 | | | | Otsuki, J. Coordination Chemistry Reviews, 2010, |
| | | | | | | 254, 2311-2341 |
| amides | various | 4 | meso | | | Otsuki, J. Coordination Chemistry Reviews, 2010, |
| | | | | | | 254, 2311-2341 |
| R | various | various | meso, | | long chain | Otsuki, J. Coordination Chemistry Reviews, 2010, |
| | | | beta | | alkyl | 254, 2311-2341 |
| | | | | | groups | |

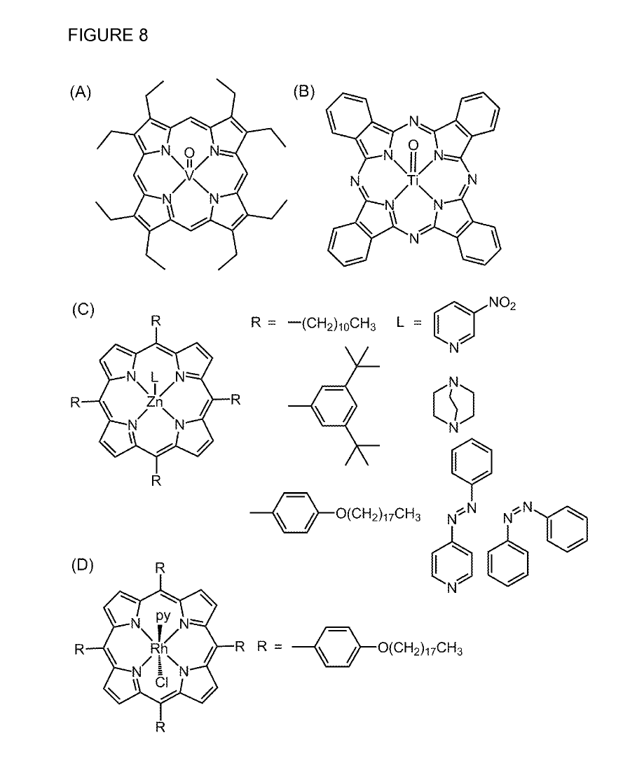

Vanadyl octaethylporphyrin (V(O)OEP) (FIG. 8(A)), titanyl phthalocyanine (Ti(O)Pc) (FIG. 8(B)), titanyl catechol phthalocyanine (Ti(cat)Pc), and aluminum chloride phthalocyanine (Al(Cl)Pc) form monolayers on HOPG with the oxo-ligand pointing up; however, because the oxo-ligand is not bidentate, its use as a binding post is limited. A variety of zinc porphyrins (ZnPor) have been shown to bind axial ligands in situ (FIG. 8(C)); however, the experimental conditions in each of the examples were designed to account for the lability of the Zn-ligand bond, which was the main challenge in forming robust complexes. Similar to the examples of square-pyramidal porphyrin monolayers with axial ligands is a report of a six-coordinate Rh-porphyrin (Rh(Cl)(Por)(py)), which also has an axial pyridine ligand pointing up (FIG. 8(D)). These challenges led to the choice of gallium octaethylporphyrin (Ga(OEP)L) as a square-pyramidal porphyrin because gallium can form covalent bonds with ligands that can act as binding posts.

The experimental probes used to deduce the composition and structure of the monolayer are largely determined by the choices for environmental conditions, substrate, and molecules. The primary means for analysis of monolayers is scanning tunneling microscopy (STM), which can be used in both ambient conditions and UHV.

Scanning tunneling microscopy imaging of octaethylporphyrin monolayers on highly oriented pyrolytic graphite

The rational design of new materials, which are of interest due to their applicability for molecular electronic devices and heterogeneous catalysis, is aided by an understanding of the intermolecular interactions that govern self-assembly of molecules on solid substrates. Molecule-molecule interactions including van der Waals forces, hydrogen bonding, and metal-ligand coordinative bonds, direct the ordering of open two-dimensional (2D) networks on a variety of substrates, which have been studied by scanning tunneling microscopy (STM). These systems of molecules on solid substrates form frameworks that are largely limited to 2D templating; extending the framework off of the surface to make three-dimensional (3D) structures may lead to new classes of materials. At present, there are few examples of 3D assemblies in the literature.

In order to design new and interesting 3D assemblies, one preferably first understands the ordering of molecules in 2D. Planar molecules such as metalloporphyrins and metallophthalocyanines are of particular interest for 3D assemblies on surfaces for a variety of reasons: they have been shown to form ordered 2D monolayers on solid substrates; they lay down with the plane of the molecule laying parallel to the surface, which allows access to coordination sites where a ligand can bind perpendicular to the plane of the porphyrin and the surface; and the 2D structures of the overlayer are tunable by varying the functional groups at the periphery of the heterocycle. The assembly of metalloporphyrins and metallophthalocyanines has been studied by scanning tunneling microscopy (STM) on a variety of substrates including HOPG, Au(111), Ag(111), and Cu(100) at low and room temperatures, and in a variety of environments, including at the solid/vacuum interface, the solid/liquid interface and the solid/air interface.

One of the simplest systems that has been studied is the 2D assembly of octaethylporphyrins on highly oriented pyrolytic graphite (HOPG) at the HOPG-liquid and HOPG-air interfaces. The majority of MOEP monolayers on HOPG studied have been imaged at the solid-liquid interface, but there are two examples of imaging at the solid-air interface.

The common theme among literature examples of MOEP monolayers on HOPG is that closely packed, ordered monolayers are formed; the molecules lay flat, with the plane of the porphyrin heterocycle oriented parallel to the substrate. Features within an STM image range in contrast from bright to dark, which corresponds to apparent heights that are tall to short. Individual MOEP molecules imaged within a domain were found to have three main features corresponding to different apparent heights within the molecule: a bright area in the region of the conjugation of the porphyrin ring; a dark area surrounding the conjugated portion of each molecule in the region of the ethyl groups; and the region at the center of the porphyrin, which ranges in appearance from darker than the conjugated ring to brighter, depending on the bias at which the monolayer is scanned.

The 2D ordering of porphyrin monolayers on HOPG are typically described using the lattice parameters. The two sides of the unit cell describing the ordering of the monolayer are x and y and Γ is the angle between them. Lattice parameters are measured from the center of one molecule to the center of an adjacent molecule.

Two basic types of 2D packing were reported for the MOEP monolayers. The majority of monolayers are reported as having distorted-hexagonal or hexagonal packing geometries. Monolayers with distorted-hexagonal packing geometry typically have lattice distances (a and b) that range from 1.2 to 1.73 nm and lattice angles (Γ) from 60° to 73°. A tetragonal packing geometry (including square and rectangular) has also been reported for NiOEP, H₂OEP, and CuOEP. Monolayers with square or distorted-square packing geometry have reported lattice distances between 1.3 and 1.4 nm and lattice angles between 90° and 93°. Oncel and Bernasek (Oncel, N.; Bernasek, S. L., Langmuir 2009, 25 (16), 9290-9295) reported that lattice parameters are dependent on the concentration of MOEP in the dosing solution; higher concentration dosing solutions produce monolayers with smaller (more tightly packed) lattice parameters.

Zou, et al. (Zou, Z. Q.; Wei, L. Y.; Chen, F.; Liu, Z. M.; Thamyongkit, P.; Loewe, R. S.; Lindsey, J. S.; Mohideen, U.; Bocian, D. F., J. Porphyrins Phthalocyanines 2005, 9 (6), 387-392.) used two-component dosing solutions to form mixed overlayers of two different MOEPs on HOPG substrates. Dosing solutions for mixed overlayers were 1:1 solutions of ZnOEP:CuOEP, ZnOEP:H₂OEP, and CuOEP:H₂OEP. They found that mixed dosing solutions led to multilayers with an alternating light and dark pattern, which they interpret as formation of a bilayer; an adlayer of one porphyrin forming on top of a monolayer of the other, with molecules of the second layer bridging the gaps between the conjugated rings of the first layer.

One-component, two-component, and three-component MOEP (M=H₂, Ni, Zn, Co) monolayers liquid dosed onto HOPG have been formed. These monolayers were dosed from benzene solutions and imaged in air. The size of arrays, the appearance of arrays at large and small scan sizes, and the features that are ascribed to individual molecules within an array, the 2D ordering of monolayers, the alignment of domains with respect to neighboring domains, the bias dependence of images, and the mobility of molecules will be discussed.

Nickel Octaethylporphyrin Monolayers on Highly Oriented Pyrolytic Graphite.

Nickel octaethylporphyrin (NiOEP) has been reported to form ordered monolayers on highly oriented pyrolytic graphite (HOPG) with the plane of the macrocycle oriented parallel to the substrate. In these studies, STM images acquired from samples prepared by dosing NiOEP from benzene solution onto HOPG show long-range ordering with domains that span tens to hundreds of nanometers. The monolayers formed using both immersion dosing and drop dosing were similar in structure. Dosing experiments produce samples that yield monolayers of sufficient quality for imaging at a better than 90% success rate, and of those samples, approximately 50% of the images were clear enough to extract lattice-parameter data. The vast majority of domains had distorted-hexagonal packing geometry, but in two experiments, rectangular packed domains were found. Images scanned at a sample bias of −450 mV for NiOEP monolayers produced images with the best resolution of the 2D structure of the overlayer. For the sake of consistency, all results discussed here are for samples formed by immersion dosing.

An STM image of the edge of a domain of NiOEP on an HOPG substrate spanning hundreds of nanometers showed that the NiOEP monolayer covered approximately 80% of the image, while a corner was bare HOPG. The contrast difference between the monolayer and the bare HOPG was due both to the height of the porphyrin as compared to the substrate, and facilitated tunneling through the conjugated heterocycle of the porphyrin. In the corner, there were some bright streaks scattered over the bare HOPG. These were attributed to either isolated single rows of porphyrin molecules or single molecules that form streaks as they move across the substrate because they are mobile at room temperature or dragged by an interaction with the tip. They were attributed as such because their height is roughly the same as the monolayer. The cross sectional profile shows that the monolayer has an apparent height of about 2.5 Å, which is similar to that observed by Ogunrinde and Hipps (2.5 Å). An undulation in the middle of the image was attributed to the substrate and was observed running through the sparsely covered HOPG in the corner. Looking closely at the porphyrin monolayer at this scan size, one could observe lines running horizontally across the domain that correspond to rows of NiOEP molecules. This is an example of Moiré fringe lines, where rows appear because of interference between the STM scan lines and the NiOEP sample. The resolution of the instrument, however, is not sufficient to observe individual molecules at this scan size. The software is only able to collect images measuring 512 pixels×512 pixels. Thus, each pixel covers 0.30 nm×0.30 nm at this scan size, which is not sufficiently small for molecular resolution. Octaethylporphyrins measure approximately 1.5 nm in diameter, but the space between adjacent molecules in the monolayer is only 0.10-0.30 nm.

The common quantitative structural characterization measurements used to describe the structures of overlayers of planar molecules on surfaces are the lattice parameters or primitive unit cell. The primitive unit cell of a NiOEP overlayer is described by two lengths, a and b, corresponding to the distances between the centers of two adjacent porphyrin molecules, and the <ab angle Γ.

STM images of NiOEP are bias dependent. A line scan of a sample imaged at a bias of −450 mV shows features that are an average of 1.6 Å tall, with the highest points on each porphyrin corresponding to the conjugated ring, and a dip at the center of each porphyrin molecule, where the Ni center is located. The cross sectional profile of the same monolayer imaged with a sample bias of +500 mV shows shorter features averaging 0.7 Å in height with a tall peak in the middle where one finds the Ni center.

Distorted-rectangular packed domains of NiOEP on HOPG were observed twice, and were well isolated from the distorted-hexagonal packed domains in a given sample. No STM images where distorted-square and distorted-hexagonal packed domains appear together could be obtained; such images were reported by Ogunrinde, et al. Images of distorted-rectangular packed domains show individual NiOEP molecules that look identical to those in the hexagonally packed arrays, but the domains have very different lattice parameters.

Free-base octaethylporphyrin monolayers on highly oriented pyrolytic graphite.

When dosed onto HOPG, free-base octaethylporphyrin (H₂OEP) forms ordered 2D monolayers with the plane of the molecule oriented parallel to the underlying substrate. STM images acquired at room temperature at a variety of sample biases show long range ordering where large arrays spanning tens to hundreds of nanometers are formed, often with multiple domains featuring different alignment relative to each other. Immersion dosing and drop dosing experiments yielded monolayers of sufficient quality for imaging at a better than 90% success rate, and approximately 50% of the images were of sufficient quality to measure the lattice parameters of the monolayers. The vast majority of the images acquired for domains of H₂OEP on HOPG show distorted-hexagonal packing geometry, but some exhibit distorted-rectangular packed domains.

Zinc Octaethylporphyrin Monolayer on Highly Oriented Pyrolytic Graphite.

Zinc octaethylporphyrin (ZnOEP) forms ordered monolayers when dosed onto HOPG. The molecular plane of adsorbed ZnOEP molecules is oriented parallel to the substrate. STM images that were acquired from samples prepared by liquid dosing from a solution of ZnOEP in benzene show long-range patterning where ZnOEP molecules aggregate to form domains ranging in size from tens to hundreds of nanometers across. In approximately 70% of experiments, samples of ZnOEP on HOPG prepared by both immersion dosing and drop dosing yielded monolayers of sufficient quality for imaging. All of the acquired images showed distorted-hexagonal-packed domains of ZnOEP. Rectangular-packed domains were not seen in any samples. Images described in the following discussion were acquired at sample biases ranging from −600 mV to −900 mV, unless otherwise noted. We chose this range of biases because they yielded the best images for the following descriptions and discussion.

Cobalt Octaethylporphyrin Monolayer on Highly Oriented Pyrolytic Graphite.

Cobalt octaethylporphyrin (CoOEP) forms ordered monolayers on HOPG with the plane of the molecule oriented parallel to the substrate. STM images acquired from the few samples that yielded monolayers with sufficient quality for imaging show long-range ordering where CoOEP molecules aggregate into domains that range in size from tens to hundreds of nanometers across. Monolayers were formed using both immersion dosing and drop dosing techniques. All samples discussed here were formed by immersion dosing. Images of CoOEP monolayers were acquired from approximately 20% of dosing experiments. For the relatively few experiments that produced visible and clearly defined arrays of CoOEP on HOPG, the domains that formed exhibited distorted-hexagonal packing geometry. Images were scanned at the sample biases that produced the best images for the purpose of the discussion.

An edge of an array of CoOEP on HOPG was observed to have a number of defects, but one could still determine the two-dimensional ordering of the monolayer.

Two-Component Mixed Monolayers on HOPG.

Mixed monolayers of H₂OEP:ZnOEP and H₂OEP:NiOEP on HOPG were studied in air at room temperature. Mixtures with varying proportions of H₂OEP:ZnOEP were dosed from benzene solutions, and formed monolayers with the molecular planes of the porphyrins oriented parallel to the substrate. For all compositions, the arrays that were formed ranged in size from tens to hundreds of nanometers across and exhibited distorted-hexagonal packing. Robust monolayers were formed using both immersion dosing and drop dosing techniques. For the sake of consistency, all of the images that will be discussed in this section were formed by immersion dosing. In approximately 80% of experiments, samples of two-component MOEP monolayers prepared on HOPG were of sufficient quality for imaging.

80% H₂OEP:20% ZnOEP.

An image (100 nm×100 nm) of a two-component monolayer dosed from an 80% H₂OEP:20% ZnOEP solution was obtained, and served as an example of long-range ordering of mixed monolayers. It shows two large, well-ordered domains and a noisy section between them. Similar to one-component monolayers, there is a ˜10° difference in the direction of the alignment of the domains with respect to each other. At this scan size, each domain is characterized by rows of MOEP molecules comprised of very bright and less bright spots. The very bright spots are attributed to the ZnOEP molecules and the rest are H₂OEP molecules, on the basis of feature height, counting statistics (vide infra), and from laser desorption/ionization mass spectrometry experiments.

The cross sectional profile shows that the ZnOEP molecules appear taller than the H₂OEP molecules by approximately 0.4 Å; however, the value for this height difference varies with scan size and scan bias (vide infra), and typically ranges from ˜0.4 Å to ˜1.0 Å. The shapes and apparent heights of the porphyrins in the cross sectional profile of the mixed monolayer have shapes and apparent heights that are comparable to those of one-component monolayers. The presence and lack of a metal in each type of porphyrin is the likely cause of this difference.

The concentrations of the two types of porphyrin were determined by counting all of the bright porphyrins and all of the less bright porphyrins in images of a number of samples at smaller scan size (40 nm×40 nm), and using those numbers to determine the percentage of each on the surface. For this sample, 19.4% of the porphyrin molecules were brighter than the rest. This number agrees well with the concentration of ZnOEP dosed onto the monolayer, suggesting that the bright spots were ZnOEP molecules and the less bright spots were H₂OEP molecules. This was also confirmed by laser desorption/ionization mass spectrometry. Hereafter, the bright porphyrins will be referred to as ZnOEP and the rest as H₂OEP. There is no apparent patterning of the ZnOEP molecules within the mixed monolayer (e.g., there is no checkerboard, which would be caused by alternating H₂OEP and ZnOEP molecules, or clustering of ZnOEP), but rather, they are randomly dispersed within the HOPG.

A 10 nm×10 nm image of the 80% H₂OEP:20% ZnOEP two-component mixed monolayer was obtained. The conjugated cores of the ZnOEP molecules appear brighter than the conjugated cores of the H₂OEP molecules while the region of the ethyl groups is approximately the same height throughout the image, suggesting that the difference in the apparent heights of the two different porphyrins is due to the presence or lack of a metal at the center. The primitive unit cell describing the 2D ordering of the two-component mixed monolayer is determined over all of the MOEP molecules in the images.

50% H₂OEP:50% ZnOEP.

An STM image of a two-component mixed monolayer dosed from a benzene solution of 50% H₂OEP and 50% ZnOEP shows a highly ordered structure with bright spots, assigned to ZnOEP molecules (vide supra), and less bright spots, assigned to H₂OEP. The average composition of ZnOEP determined by counting was 47.8% (Table 2.5). The two-dimensional distorted-hexagonal packing of the monolayer is clear, and it is clear from the cross sectional profile which molecules are ZnOEP and which are H₂OEP. The cross sectional profile shows ZnOEP molecules that are taller than the H₂OEP molecules by approximately 0.4 Å, which is similar to the relative heights found. There does not appear to be any specific order to the distribution of ZnOEP and H₂OEP molecules within the monolayer, which is consistent for all imaging experiments of 50% H₂OEP:50% ZnOEP monolayers.

In a 10 nm×10 nm scan size image of a two-component mixed monolayer of 50% H₂OEP:50% ZnOEP where the ZnOEP molecules clearly appear brighter than the H₂OEP molecules, the cross sectional profile shows ZnOEP molecules that are ˜1 Å taller than the H₂OEP molecules, which is a larger height difference than in the image recorded at a larger scan size. The heterocyclic portion of both molecules is brighter than the region at the periphery of the porphyrin ring where the ethyl groups are located, which is similar to that observed for one-component monolayers of MOEP on HOPG. The primitive unit cell is determined using all of the MOEP molecules.

20% H₂OEP:80% ZnOEP.

An image of a two-component mixed monolayer (40 nm×40 nm) (FIG. 2.28) dosed onto HOPG from a benzene solution of 20% H₂OEP and 80% ZnOEP consists of highly ordered bright features with distorted-hexagonal packing geometry. The bright spots are assigned to the conjugated core of individual porphyrin molecules, and the dark space between them is assigned to the region of the ethyl substituents at the periphery of the heterocyclic ring. It is difficult to distinguish which porphyrin molecules are ZnOEP and which are H₂OEP in this image due to the relatively large scan size (40 nm×40 nm), but there are some areas in the image that are clearer than others. For example, the cross sectional profile through the row of porphyrins at the top of the image shows two types of features that differ in height by approximately 0.6 Å. The tall peaks are assigned to ZnOEP and the short peaks are assigned to H₂OEP. The average composition of ZnOEP determined by counting was 74.9%.

A smaller scan size image (15 nm×15 nm) of the 20% H₂OEP:80% ZnOEP two-component mixed monolayer better illustrates the height differences in the molecules. The ZnOEP molecules are brighter than the H₂OEP molecules. A cross sectional profile shows that the ZnOEP molecules are taller than the H₂OEP molecules by 1.4 Å-2.4 Å. The variation of apparent height from one sample to another is similar to that seen for images of the mixed monolayers composed of 50% H₂OEP and 50% ZnOEP. The primitive unit cell is determined for all of the MOEP molecules.

50% H₂OEP:50% NiOEP.

The STM image of a two-component mixed monolayer immersion dosed from a benzene solution of 50% H₂OEP and 50% NiOEP shows a highly ordered structure of porphyrinic features, assigned to both H₂OEP and NiOEP. The two-dimensional distorted-hexagonal packing of the monolayer is clear; however, it is unclear from the either visual analysis of the image or the cross sectional profile which molecules are H₂OEP and which are NiOEP. The overall appearance of the overlayer is very similar to both H₂OEP and NiOEP one-component monolayers, and the lattice parameters are also similar.

Three-component mixed monolayers of H₂OEP, ZnOEP and NiOEP on HOPG.

A three-component mixed monolayer on HOPG, dosed from a solution of 20% H₂OEP, 40% ZnOEP, and 40% NiOEP, was studied in air at room temperature. Robust monolayers were formed using both immersion dosing and drop dosing techniques. The arrays that were formed ranged in size from tens to hundreds of nanometers across with the molecular planes of the porphyrins oriented parallel to the substrate. For the sake of consistency, all of the images that will be discussed in this section were formed by immersion dosing. In approximately 90% of experiments, samples of three-component MOEP monolayers prepared on HOPG yielded monolayers of sufficient quality for imaging. All of the domains had distorted-hexagonal packing geometry.

The STM image of a three-component mixed monolayer dosed onto HOPG from a 20% H₂OEP, 40% ZnOEP, and 40% NiOEP benzene solution showed the two-dimensional ordering of two adjacent domains spanning hundreds of nanometers.

A (30 nm×30 nm) image of a three-component mixed monolayer shows two main types of porphyrins. One type of porphyrin is bright and has a noisy, ill-defined border as indicated by the porphyrin in the squares. This type of porphyrin covers 35.6% of the surface, and because it looks very similar to ZnOEP molecules in two-component mixed monolayers of ZnOEP and H₂OEP it has been assigned as such. There does not appear to be any secondary ordering of these molecules within the monolayer; rather, there seems to be a random distribution of ZnOEP molecules. The remaining 60% of the monolayer is composed of porphyrins like those in the circles, which have a toroidal shape with a dark center surrounded by a bright ring. The appearance of these porphyrins is similar to porphyrins in two-component mixed monolayers of H₂OEP and NiOEP, and the two types of porphyrin are not distinguishable from one another.

The height difference between ZnOEP and H₂OEP or NiOEP is illustrated in a 10 nm×10 nm scan size image of the three-component mixed monolayer. The cross sectional profile shows porphyrins of two different heights differing by approximately 0.6 Å, which is within the 0.4 Å to 1 Å range of the height difference between ZnOEP and H₂OEP in the majority of samples of two-component mixed monolayers.

In one of the imaging experiments of a three-component mixed monolayer formed by dosing HOPG with a benzene solution composed of 20% H₂OEP, 40% ZnOEP, and 40% NiOEP, a particularly vivid instance of the mobility of MOEP molecules on HOPG at room temperature was observed. Twelve consecutively recorded images showing the same portion of the edge of a monolayer where two domains come together were obtained. In each successive image, molecules are added to one domain or the other until the space between the domains is filled in. This example will be referenced when discussing the driving forces for the mobility of porphyrin molecules on HOPG at room temperature in the discussion section.

Typical STM images of one-, two- and three-component monolayers composed of H₂OEP, NiOEP, CoOEP and ZnOEP consist of an organized structure of closely packed disc-shaped molecules in arrays between tens and hundreds of nanometers across. Two types of packing geometry were found: distorted-hexagonal packing was found for all porphyrins; and distorted-rectangular packing was found for only NiOEP and H₂OEP. Even for these two porphyrins, distorted-rectangular-packed arrays are rarely found. Hexagonal packed arrays of all of the MOEPs have lattice spacings that are the same within experimental error.

Like images acquired by Ogunrinde, et al. for NiOEP on HOPG under similar conditions, our images show two types of domains; a distorted-hexagonal-packed structure, and a distorted-rectangular-packed structure. The lattice parameters found for distorted-hexagonal-packed domains of monolayers of NiOEP on HOPG imaged in air agree well with those for NiOEP at the 1-phenyloctane/HOPG interface found by Oncel, et al.(Oncel, N.; Bernasek, S. L., Langmuir 2009, 25 (16), 9290-9295) and are smaller than those found by Ogunrinde, et al. Oncel, et al. attributed differences in lattice parameters to the concentration of the dosing solutions, where higher concentrations lead to more densely packed monolayers. The dosing concentration and packing density for the NiOEP monolayers that we found (2.5±10⁻⁴ M, 5.57×10¹³ molecules/cm²) is between those reported by Oncel, et al. (5±10⁻⁴ M, 5.62×10¹³ molecules/cm²) and Ogunrinde, et al. (5±10⁻⁵ M, 4.5×10¹³ molecules/cm²), which is in agreement with the explanation presented by Oncel, et al. The lattice angle that we found for the rectangular-packed arrays of NiOEP is similar to Ogunrinde, et al.; however, they do not present lattice distances, so we lack a point of comparison.

For monolayers of H₂OEP on HOPG imaged in air, we found a distorted-hexagonal-packed structure, which differs from that found by Zou, et al. at the o-dichlorobenzene/HOPG interface. Zou, et al. reported that they observed only a rectangular-packed structure for H₂OEP. Although the lattice parameters they report for the rectangular-packed monolayers agree well with those we found for the same packing geometry, we only found one example of the rectangular structure. The vast majority of our arrays were composed of monolayers of H₂OEP with distorted-hexagonal packing geometry.

For monolayers of ZnOEP on HOPG imaged in air, a distorted-hexagonal-packed structure was found with lattice spacings that differ from those found by Zou, et al. at the o-dichlorobenzene/HOPG interface. In two separate reports, Zou, et al. reported smaller unit cells for ZnOEP. It is also interesting to note that the two lattice parameters that they report differ significantly from each other.

Monolayers of CoOEP yielded quality monolayers less often than ZnOEP, NiOEP and H₂OEP. The lattice parameters for monolayers of CoOEP were found to be smaller than those found for any of the other three porphyrins we imaged.

For STM images of two-component monolayers of H₂OEP and ZnOEP, we have only found distorted-hexagonal packed domains. For monolayers formed from dosing solutions composed of 80% H₂OEP:20% ZnOEP, 50% H₂OEP:50% ZnOEP, and 20% H₂OEP:80% ZnOEP the lattice distance found for a is closer to b, than for one-component monolayers. In two-component monolayers of H₂OEP and ZnOEP, ZnOEP molecules appear brighter than H₂OEP molecules. The ZnOEP molecules are randomly interspersed among the H₂OEP. The compositions of the monolayers as determined by counting brighter and darker molecules agree well with the composition of dosing solution, which leads us to conclude that both ZnOEP and H₂OEP have similar affinities for adsorption on HOPG.

A three-component monolayer composed of 20% H₂OEP, 40% ZnOEP, and 40% NiOEP was also dosed onto HOPG. Similar to the two-component overlayers of ZnOEP and H₂OEP, the ZnOEP appears brighter than the other porphyrins. However, the H₂OEP and NiOEP have nearly identical heights. By counting, the ZnOEP composition of the mixed monolayer is generally similar to the composition of the dosing solution.

The ease of preparing and imaging one-component monolayers of MOEP decreases in the order: NiOEP>H₂OEP>ZnOEP>CoOEP. This may due to the strength of molecule-substrate interactions, but is not likely due to molecule-molecule interactions. We come to this conclusion because in two- and three-component monolayers, there does not appear to be any secondary ordering. Rather, there is a random distribution of molecules throughout the monolayer.

The rows of neighboring domains of MOEP are often rotated with respect to each other. This type of rotational alignment with respect to neighboring domains is similar to that reported for nickel tetraphenylporphyrin and NiOEP on Au(111). The difference between the directional alignments of the rows of neighboring domains is typically on the order of 10°.

Consecutively acquired images of porphyrin monolayers on HOPG that change from one image to the next illustrate that the molecules are mobile on HOPG at room temperature, but it is unclear whether the mobility is STM tip-induced dragging/pushing of the molecules, or if the mobility is due to thermal motion. A similar phenomenon is seen by Scudiero, et al. (Scudiero, L.; Hipps, K. W., Journal of Physical Chemistry C 2007, 111 (47), 17516-17520.), where they show that tip-induced mobility is possible. They use the tip to selectively remove molecules from the monolayer, which illustrates that at certain biases, there is sufficient attraction between the tip and the molecules to move molecules around the surface. We also see streaks of MOEP in areas of bare HOPG, which is consistent with Scudiero, et al. They assign the streaks to be individual molecules that possess the thermal energy to be mobile on HOPG at room temperature.

Conventional LDI-TOF MS and MALDI-TOF MS Measurements of Octaethylporphyrins.

The mass spectra of octaethylporphyrins prior to deposition on HOPG were acquired using a standard 100 well stainless steel sample plate with matrix (MALDI-TOF MS) and without matrix (LDI-TOF MS). For LDI-TOF MS samples, 0.5 μL of a porphyrin dosing solution (2.5×10⁻⁴ M in benzene) was deposited directly onto the sample plate, the solvent was allowed to evaporate in air, and the sample plate was immediately loaded into the instrument to record spectra at a variety of laser powers. For MALDI-TOF MS samples, 0.5 mL of a solution composed of 10 μg of matrix (2-hydroxy-1-naphthoic acid) dissolved in 1 mL of either acetonitrile or methanol was deposited onto the sample plate and the solvent was allowed to dry in air. Then 0.5 μL of a porphyrin dosing solution (2.5×10⁻⁴ M in benzene) was deposited on top of the matrix, the solvent was allowed to exaporate in air, and the sample plate was immediately loaded into the instrument to record spectra at a variety of laser powers. LDI-TOF MS and MALDI-TOF MS measurements were performed on a Voyager DE-PRO Biospectrometry mass spectrometer (Applied Biosystems, Framingham, Mass.). A nitrogen laser (λ=337 nm) was used as the source for desorption/ionization. All spectra were acquired with a 20 kV accelerating voltage, using the positive reflector mode. Extraction delay was 100 ns, and 100 laser shots were applied for each spectrum. Spectra were saved as ASCII files using the Data Analysis program in order to process data using Excel.

LDI-TOF MS Measurements of Octaethylporphyrin Monolayers on HOPG.

After imaging the porphyrin monolayers on HOPG with the STM, samples were loaded onto a specially designed LDI-TOF MS plate with a sample trough in the center machined to accommodate three mounted HOPG substrates. The depth of the well positions the surface of a 1 mm thick HOPG sample even with the top of the sample plate, which is the reference plane for the time-of-flight experiment (vide infra). LDI-TOF MS measurements were performed on a Voyager DE-PRO Biospectrometry mass spectrometer (Applied Biosystems, Framingham, Mass.). A nitrogen laser (λ=337 nm) was used as the source for desorption/ionization. All spectra were acquired with a 20 kV accelerating voltage, using the positive reflector mode. Extraction delay was 100 ns, and 100 laser shots were applied for each spectrum. Spectra were saved as ASCII files using the Data Analysis program in order to process data using Excel. Unless otherwise noted, discussions of the molecular ion refer to the nominal molecular weight peak, which is defined as “[the molecular weight] calculated by summation of the atomic masses of the [most abundant] isotope of each element composing the molecule,” using integer values (i.e., mass defect is not taken into account).

When the LDI-TOF laser was left at a static position during mass spectra acquisition of the porphyrin monolayers, the signal-to-noise ratio of the data was very poor. This is attributed to the absence of porphyrin molecules on the substrate after ablation of the monolayer by a laser pulse. In order to increase the signal-to-noise ratio, the laser was rastered across the sample during acquisition using a joystick controller to expose “fresh” monolayer to the laser. The relationship between signal-to-noise improvement and scan speed could not be quantified in this study due to inhomogeneous monolayer coverage and a lack of precise control over the rastering speed by the operator of the instrument.

Because the degree of fragmentation observed in the spectra varies with laser power and affects the data analysis, spectra were collected for a variety of laser powers between 1400 (low power) and 1800 (high power) on an arbitrary scale of 0-3000 for the instrument. On this scale, the minimum laser power required for desorption/ionization increases over time (month-to-month) as the instrument's laser degrades. The data acquired here were recorded over a sufficiently short period of time (approximately one month) that laser degradation was considered negligible; however, samples recorded approximately six months later required laser powers between 1700 and 2000 to achieve spectra with similar signal-to-noise ratio and fragmentation.

The LDI-TOF mass spectrometer is mass-calibrated for analytes desorbed/ionized from the surface of a standard stainless steel sample plate at a specific distance from the detector. Analytes lying above and below the level of the calibrated sample plate will show smaller and larger masses, respectively. Small differences in the thickness of the sample disk and HOPG substrate change the time-of-flight path length to the detector, thus changing the observed mass. Because the thickness of the substrate changes each time the HOPG is cleaved, each spectrum had to be individually mass-calibrated. This was accomplished by applying a constant m/z correction to the data so that the features matched a reference spectrum of that MOEP deposited on a standard stainless steel LDI-TOF MS sample plate. The LDI-TOF MS plate was designed such that the surface of a piece of 1 mm thick HOPG substrate mounted on a stainless steel sample puck would be level with the sample plate, and thus, require very little calibration. The monoisotopic masses (calculated molecular mass including mass defect) are used in the discussion of mass accuracy because it is a more exact description of the mass than the nominal molecular weight. Isotope patterns of all three are similar, as expected. The 1 mm thick substrate has much higher mass accuracy (Δm/z=0.0114, ˜19 ppm) than the 2 mm thick substrate (Δm/z=1.9807, ˜3332 ppm). Since each monolayer here is entirely composed of one or more OEPs of known mass and ionization properties, those molecules are used as the internal standard during method development.

The composition of ZnOEP, H₂OEP and NiOEP molecules in two- and three-component mixed monolayers was determined using the LDI-TOF MS spectral data. In order to suppress overlap of the fragment ion peaks for the different porphyrins, it was preferable to use spectra acquired at low laser power. For a given component of a mixed monolayer (ZnOEP, H₂OEP, or NiOEP), the intensities of all of the peaks in the isotope pattern of a particular ion (molecular or fragment) were summed to produce a value for the abundance of that ion. The resulting values for the abundances of fragment ions were added to the molecular ion abundance to give molecular composition of that component in the spectrum. The molecular compositions of all components of a monolayer were used to evaluate the percentage of surface composition for each porphyrin. This was done for each dosing mixture: 80% H₂OEP:20% ZnOEP, 50% H₂OEP:50% ZnOEP, 20% H₂OEP:80% ZnOEP, and 20% H₂OEP:40% ZnOEP:40% NiOEP.

Determination of the Ionization Products of Octaethylporphyrin in LDI-TOF MS and MALDI-TOF MS Experiments.

In order to accurately apply m/z corrections to LDI-TOF spectra of monolayers of octaethylporphyrin on HOPG substrates, which have varying thickness, it was necessary to understand how the analytes ionize. LDI-TOF MS and MALDI-TOF MS measurements with samples loaded on a stainless steel sample plate were used to determine whether the octaethylporphyrin ionizes by loss of an electron to produce the MOEP.⁺ radical cation, by gaining H⁺ to produce [MOEP+H]⁺, or by another pathway. Four conditions were explored: LDI-TOF MS at low laser power (relative to the threshold for detection) and high power (significant fragmentation); and MALDI-TOF MS (with 2-hydroxy-1-naphthoic acid, HNA, as a matrix) at low and high laser powers. At low laser powers, H₂OEP, NiOEP and ZnOEP were found to ionize primarily as MOEP.⁺ both with and without matrix; however, at higher laser powers mass spectra show more complex ionization products. Samples were calibrated using HNA as an internal standard.

Mass spectra of NiOEP acquired with matrix differ from those acquired without matrix. At low laser power without matrix NiOEP ionizes by loss of an electron to form the NiOEP.⁺ radical cation (m/z=590), as indicated by the fact that the observed isotope pattern agrees well with prediction. Zinc and magnesium tetraphenylporphyrin have also been reported to ionize to form M(OEP).⁺ by LDI-TOF MS. In the presence of matrix (HNA) at low laser power (near the ionization threshold of the matrix), NiOEP ionizes primarily by loss of an electron to produce the NiOEP.⁺ radical cation, with a small amount ionizing by addition of H. This conclusion is based on the fact that the observed isotope pattern does not match that predicted for NiOEP.⁺, but contains contributions from a species shifted one mass unit higher. The spectrum can be fit as a sum of the theoretical spectra of NiOEP.⁺ and [NiOEP+H]⁺. At higher laser powers with and without matrix present, ionization of NiOEP was more complex, and the isotope pattern does not agree with the predicted isotope patterns of NiOEP.⁺, [NiOEP+H]⁺, or a linear combination of the two. The isotope patterns of NiOEP acquired at high laser power could be nearly matched only by applying linear combinations of the isotope patterns of five ions: NiOEP.⁺, [NiOEP−H]⁺, [NiOEP+H]⁺, [NiOEP+2H]⁺ and [NiOEP+3H]⁺.

Mass spectra of H₂OEP acquired with matrix are very similar to those acquired without matrix at both low and high laser powers. At low laser power (below the ionization threshold of the matrix) H₂OEP ionizes primarily by loss of an electron to produce the H₂OEP.⁺ radical cation (m/z=534), as indicated by the agreement between observed and predicted isotope patterns. At higher laser powers, ionization of H₂OEP was found to be more complex, and the isotope pattern does not agree with the predicted isotope patterns of H₂OEP.⁺, [H₂OEP+H]⁺, or a linear combination of the two. The isotope patterns of H₂OEP acquired at high laser power was matched by applying linear combinations of the isotope patterns of four possible ions: H₂OEP.⁺, [H₂OEP+H]⁺, HOEP⁺, and [H₂OEP+2H]⁺.

Fragment ions for NiOEP, ZnOEP, and H₂OEP form by loss of a methyl group from the β-ethyl substituents. These ions are located at approximately m/z=(molecular ion)−n(15) where n is the number of methyl groups lost (n=1-8). Although there is evidence for fragmentation beyond the loss of eight methyl groups, this only occurs at very high laser powers that are well beyond those used in this study. The isotope patterns of fragment ions at all laser powers, with or without matrix, are similar to those for molecular ions of OEPs at higher laser powers, showing complex ionization products.

In addition to fragmentation of the porphyrin ring, demetalation can occur under certain conditions. In the presence of matrix, the peak at m/z=534 is evidence for demetallation of the porphyrin similar to that found for MgTPP with acidic matrices, whereas without matrix there is no demetallation peak. Demetallation was not obtained for ZnOEP under similar conditions, which is consistent with literature reports for MALDI-TOF of other Zn-porphyrins.

LDI-TOF MS of One-Component MOEP Monolayers on HOPG.

Typical STM images of one-component monolayers of free-base octaethylporphyrin (H₂OEP), nickel octaethylporphyrin (NiOEP), and zinc octaethylporphyrin (ZnOEP), show patterns arising from ordered, closely packed molecules that make up arrays on the order of tens to hundreds of nanometers across. As described elsewhere herein, these monolayers have distorted-hexagonal or distorted-rectangular packing geometry. The appearance of features in the images depends greatly on the sample bias at which monolayers were scanned; thus, monolayers of different composition can look very similar to each other. The cross sectional profiles of H₂OEP, NiOEP and ZnOEP monolayers show features ranging from ˜1.2 Å to ˜1.6 Å in height. The primitive unit cells for each of the three samples have similar dimensions and angles. Thus, it would be difficult to identify the composition of the monolayer on the basis of the STM data provided.

In situ LDI-TOF MS was employed as an analytical tool to characterize the composition of octaethylporphyrin monolayers on HOPG. The spectra obtained from monolayers of H₂OEP on HOPG are typical of these materials. At low laser power, the spectrum is dominated by a molecular ion peak (m/z=534) whereby ionization occurs primarily by loss of an electron to give MOEP.⁺ radical cation. The isotope pattern of the molecular ion agrees with the predicted spectrum. Weak signals due to fragment ions are also present. There is no evidence in the m/z≦267 region for H₂OEP²⁺ or doubly charged fragments.

While at low laser power there is little fragmentation of the H₂OEP⁺ ion, at higher laser powers up to eight fragment peaks are observed. These fragments are assigned to loss of methyl units from the eight ethyl groups at the β-position of the porphyrin. At low laser power, the intensities of fragment peaks in the spectrum decrease with more lost methyl groups. As laser power is increased, the intensities of the ions with higher fragmentation increase. The increase in complexities of the isotope patterns of ions at higher fragmentation is likely due to overlap of fragments with different ionization pathways, as previously discussed for H₂OEP.

Mass spectra of one-component monolayers of NiOEP and ZnOEP on HOPG recorded at low laser power show that, for each porphyrin, the presence of a polyisotopic metal atom results in a molecular ion peak, which exhibits a unique isotope pattern (inset) that, together with its m/z ratio, allows fingerprinting of the monolayer being studied. In each case, the molecular ion isotope pattern is consistent with MOEP.⁺. This is similar to the situation observed for the compounds on a standard stainless steel sample plate.

3.3.3. LDI-TOF MS of Two-Component Mixed Monolayers of MOEP on HOPG.

Mixtures of octaethylporphyrin compounds form monolayers on HOPG whose structures resemble those of pure porphyrins. The mixed monolayers are compositionally disordered; ZnOEP molecules, which appear brighter than H₂OEP molecules under these imaging conditions, are randomly distributed. The apparent heights for each type of molecule correlate well with those found in their one-component monolayers. Under these dosing conditions, there does not appear to be a preference for depositing one type of porphyrin over the other. The compositions of the two types of porphyrin in the monolayers were determined by counting bright and less-bright features, and the resulting percentages of each on the surface are similar to those of the dosing solution.

LDI-TOF mass spectra of two-component monolayers formed by dosing HOPG with a solution of H₂OEP and ZnOEP show molecular ions at m/z=534, which corresponds to H₂OEP.⁺, and at m/z=596, which corresponds to ZnOEP.⁺. The isotope patterns of the molecular ions agree with prediction. Fragment ions are present corresponding to the elimination of a terminal methyl group from one of the ethyl β-substituents. The relative intensities of the two molecular ion peaks in each spectrum are seen to be in qualitative agreement with the dosing composition. Specifically, the spectra of the two 80:20 mixtures are dominated by the molecular ion of the 80% compound, whereas for the 50:50 mixture, the two molecular ions are of roughly equal intensity.

The number of peaks observed in the LDI-TOF mass spectra of mixed monolayers is sensitive to laser power. At low laser power, there is minimal overlap between fragment ions of ZnOEP and the H₂OEP parent ion. At higher laser powers more fragmentation occurs, and some of the ZnOEP fragment ions overlap with the H₂OEP molecular ion and subsequent fragments. Thus, the ease with which the components can be identified varies with laser power, with lower powers being preferred.

The composition of ZnOEP and H₂OEP molecules in two-component mixed monolayers was determined using the LDI-TOF MS spectral data for each of three dosing mixtures: 80% H₂OEP:20% ZnOEP, 50% H₂OEP:50% ZnOEP and 20% H₂OEP:80% ZnOEP. In order to suppress fragment ion overlap of the two porphyrins, it was preferable to use spectra acquired at low laser power. A graph comparing the calculated percentage of each porphyrin in the mixed monolayer (determined by LDI-TOF MS) versus percentage in the dosing solution shows that the monolayer composition is directly proportional to the composition of the dosing solution.

In order to control for possible substrate- and laser power-induced differences in experiments, two dosing/imaging/LDI-TOF MS experiments were performed on each of three different HOPG substrates, and in each experiment mass spectra were acquired using each three different (low) laser powers (1400, 1450, and 1500). There is a wide range of data for each dosed composition, but the regression line shows that there is a strong correlation (r²=˜0.94) between the composition dosed and the composition calculated from experiment.

If it is assumed that the composition of the mixed monolayer should, on average, be identical with that of the dosing solution, then an examination of the experimental variables that affect the spread of the data and the accuracy of the LDI-TOF MS measurements with respect to the dosing solution composition provides a means to optimize the measurement. A series of control experiments was performed to determine which variables are sensitive to changes in the experimental conditions. The control experiments were performed in three ways: three sample disk/HOPG surfaces were used to account for substrate variations; three (relatively low) laser powers were employed to control the signal-to-noise ratio and ion overlap in spectra; and the compositions were determined using only the molecular ions of each component.

The data for the control experiments show that the measurements are reproducible over all three substrates and using multiple laser powers. There is some scatter to the calculated proportions of H₂OEP, but these data are all grouped around the values for the proportion of H₂OEP dosed onto the HOPG substrates. The only immediately apparent trend in the control experiments is that the calculated proportion of H₂OEP increases with increasing laser power. For the samples prepared using dosing solutions composed of 20% and 80% H₂OEP, the average proportion of H₂OEP in the monolayer calculated by LDI-TOF-MS converges on the proportion of H₂OEP in the dosing solution as the laser power is increased. However, for the samples prepared using dosing solutions composed of 50% H₂OEP, the calculated average proportions of H₂OEP in the monolayer diverge from the proportion of H₂OEP in the dosing solution. We are hesitant to draw any conclusions from this observation because it may be due to the small sample size.

For control experiments where the surface composition was determined using only the molecular ions, the averages are similar to those calculated using all molecular and fragment ions; however, only low laser powers were used for this purpose. Thus, we can conclude that at the low fragmentation limit, concentrations can be calculated with confidence using either the molecular and fragment ions or using only the molecular ion. At higher laser powers, however, the ratio of the molecular ions will be more dependent on fragmentation of the analytes, and the proportions of the analytes on the surface determined by LDI-TOF MS using only the molecular ions may prove less accurate.

LDI-TOF MS of Three-Component Mixed Monolayers of MOEP on HOPG.

For a three-component mixed monolayer formed by dosing HOPG with a benzene solution composed of 20% H₂OEP, 40% ZnOEP, and 40% NiOEP, STM images show arrays of the MOEPs organized into distorted-hexagonal-packed domains with the plane of the molecules oriented parallel to the substrate. Similar to images of two-component monolayers of ZnOEP and H₂OEP, at a sample bias of −800 mV the ZnOEP in three-component monolayers appears brighter, thus taller than H₂OEP and NiOEP, which have nearly similar heights. By counting bright features, the ZnOEP composition in the monolayer is found to be 35.6%, which is close to the 40% composition in the dosing solution.

The LDI-TOF MS of this three-component monolayer shows molecular ions at m/z=534, corresponding to H₂OEP.⁺, at m/z=591, corresponding to NiOEP.⁺, and at m/z=596, corresponding to ZnOEP.⁺. The isotope patterns of the molecular ions agree prediction. Fragment ions are also present, corresponding to the elimination of a terminal methyl group from one of the ethyl β-substituents at the periphery of the porphyrin heterocycle.

The monolayer composition determined by LDI-TOF MS differs from the composition of the dosing solution. As determined by LDI-TOF MS the average compositions of H₂OEP (13.5%±4.4%) and ZnOEP (32.5%±7.4%) are well below their compositions in the dosing solution, and NiOEP (54.1%±6.1%) is well above its composition in the dosing solution.

Typical LDI-TOF MS spectra of one-component MOEP monolayers desorbed from HOPG at low laser intensity are dominated by molecular ions with isotope patterns that are consistent with prediction. This allows for use of LDI-TOF MS as means of independently probing the surface composition of monolayers, which have previously been imaged by STM.

Fragment ions are present at higher laser intensities, corresponding to the elimination of one to eight methyl groups from the β-ethyl substituents at the periphery of the porphyrin heterocycle. Although isotopic distributions of the molecular ion peaks agree with predicted values, the same cannot always be said of the isotopic distributions of fragment ion peaks. This is likely due to a complex mixture of ionized products with similar masses, e.g., [MOEP-CH₃]⁺+[MOEP-CH₃+H]⁺, which form in the plume after the sample has been excited. One of the limitations to assigning LDI-TOF mass spectra is that the chemistry that takes place within the plume is not well understood and difficult to study.

LDI-TOF mass spectra of two-component monolayers of H₂OEP and ZnOEP show two molecular ions: one corresponds to H₂OEP.⁺ at m/z=534; and one corresponds to ZnOEP.⁺ at m/z=596. The isotopic distributions of both ions agree with prediction, which makes LDI-TOF MS a useful tool to independently probe the surface composition of mixed monolayers that have previously been imaged by STM. The composition of two-component monolayers was determined using LDI-TOF MS spectra. The spectra used for this purpose were acquired at low laser intensity to minimize fragment ion overlap. The summation of the peak heights of the molecular ions and all fragment ions was used to evaluate the percentage of surface composition for each porphyrin for each of three dosing mixtures: 80% H₂OEP:20% ZnOEP, 50% H₂OEP:50% ZnOEP and 20% H₂OEP:80% ZnOEP. It is evident that there is a relationship between the dosed composition and the adsorbed composition on the surface. The wide distribution of points for the LDI-TOF MS data acquired for samples from each dosing mixture shows that the method is inaccurate for single measurements. When taken as the average of many data points at each dosing composition, however, the composition calculated from LDI-TOF MS spectra approaches the composition of the dosing solution.

The LDI-TOF MS of three-component monolayers composed of 20% H₂OEP, 40% ZnOEP, and 40% NiOEP shows three parent ions, one for each of the three metalloporphyrins; the isotope patterns agree with predicted values. By counting bright molecules in the images, the ZnOEP composition in the overlayer is found to be 35%. As determined by LDI-TOF MS the composition of ZnOEP agrees well with the composition of the dosing solution at ˜39%, however the compositions of H₂OEP and NiOEP are ˜12% and ˜49%, respectively. This discrepancy from the dosing solution is likely due to the differences in the ionization potential of the three porphyrins in the plume. From this information it is clear that LDI-TOF MS is not a quantitative method for determining surface composition of mixed monolayers. It can, however, be used as a semi-quantitative method. In order to use it this way, a calibration curve can be created for a particular system, and from that, an estimate of surface composition can be made using LDI-TOF MS spectra.

Confocal Raman Microscopy of Porphyrin Monolayers on HOPG

Due to their strong electronic absorption bands (c 104-10⁶M⁻¹ cm⁻¹) their Raman spectra exhibit pronounced resonance enhancement effects. Given the importance of Raman spectroscopy as a sensitive probe of molecular structure, it seemed desirable to apply it to study porphyrin monolayers on HOPG, especially since the resonance enhancement might overcome the weak signals for such a low-concentration material (˜10¹³ molecules/cm²).

Confocal Raman microscopy is a method for in situ study of the composition of porphyrin monolayers on HOPG. One- and two-component monolayers of octaethylporphyrins (MOEP; M=H₂, Ni, Zn, Co) dosed onto highly oriented pyrolytic graphite (HOPG) substrates were imaged by STM in air at room temperature, and probed by both laser-desorption ionization time-of-flight mass spectrometry (LDI-TOF MS) and confocal Raman microscopy.

Confocal Raman Microscopy Measurements.

Confocal Raman Microscopy Measurements of One-Component Monolayers of Metallooctaethylporphyrin on HOPG.

STM images of one-component monolayers of free-base octaethylporphyrin (H₂OEP), nickel octaethylporphyrin (NiOEP), zinc octaethylporphyrin (ZnOEP), and CoOEP, show an organized structure of closely packed bright features making up arrays spanning tens to hundreds of nanometers across. These monolayers have distorted-hexagonal or distorted-rectangular packing geometry. The appearance of features in the images depends greatly on the sample bias at which monolayers were scanned; thus, monolayers of different porphyrins can look very similar to each other. The cross sectional profiles of H₂OEP, NiOEP and ZnOEP monolayers typically show features ranging from ˜1.2 Å to ˜1.6 Å in height. The primitive unit cells for each of the three samples have similar dimensions and angles. Thus, it is difficult to identify the composition of the monolayer on the basis of the STM data alone.

After imaging with STM and acquiring LDI-TOF MS data of the monolayers, samples were taken to the Center for Nanoscale Materials at Argonne National Laboratory to measure the Raman spectra with a confocal Raman microscope. The microscope provides several choices for excitation wavelength. Because the porphyrin analyte is present in such low concentration (˜10¹³ molecules/cm²) it is desirable to select excitation wavelengths that coincide with porphyrin electronic absorption bands to take advantage of the resonance Raman effect.

The three excitation wavelengths used to produce the Raman spectra of the monolayers and thin films are 441.6 nm (blue), 514.5 nm (green), and 632.8 nm (red). For each of the four porphyrins, the 441.6 nm excitation wavelength lies at the extreme red tail of the Soret band, which allows for pre-resonance enhancement of the Raman scattering; however, the overlap of the excitation wavelength with the Soret tail is different for each molecule, so the magnitude of the enhancement may differ for each sample. The Raman spectral window associated with the 441.6 nm excitation wavelength does not overlap with the fluorescence of any of the molecules, so these spectra should not have background contributions from fluorescence. The 514.5 nm excitation wavelength lies near the center of the Q₁ absorption bands of NiOEP and CoOEP, and at the red and blue tails of the Q₁ absorption bands of H₂OEP and ZnOEP, respectively.

A trade-off associated with excitation into the lower-energy Q-absorption bands is that, while overlap with the band is stronger than for the Soret band, fluorescence could coincide with the Raman scattering signal for H₂OEP and ZnOEP. The resonance enhancement of the NiOEP and CoOEP Raman spectra using a 514.5 nm excitation wavelength seems to be stronger than that for H₂OEP and ZnOEP, which is consistent with the greater overlap with the Q₁ absorption band. The Raman shift window associated with the 514.5 nm excitation wavelength overlaps with the blue tail of the fluorescence spectrum of ZnOEP, which may account for worse signal-to-noise in the ZnOEP Raman spectrum. The fluorescence of ZnOEP and H₂OEP also overlap with the Raman shift window associated with the 632.8 nm excitation available with the instrument, causing saturation of the detector, thus this wavelength was not useful for Raman experiments with these molecules.

Typical Raman spectra of a freshly cleaved HOPG substrate and a one-component NiOEP monolayer measured using excitation wavelengths of 514.5 nm and 441.6 nm are dominated by large bands at 1580 cm⁻¹, 2440 cm⁻¹, and 2727 cm⁻¹, which agree with literature values for the HOPG substrate. In the region between 600 cm⁻¹ and 1700 cm⁻¹ there are a number of much weaker bands attributed to the NiOEP molecules of the monolayer. Relative to the HOPG band at 1580 cm⁻¹, the NiOEP peaks are much stronger in the spectrum acquired using the 441.6 nm excitation wavelength than in the spectrum acquired using the 514.5 nm excitation wavelength. Because these spectra were acquired for different samples, it is unknown whether this is due to better monolayer coverage over the substrate, to enhanced resonance scattering of the porphyrin upon 441.6 nm excitation, or to other effects.

The Raman spectra of a NiOEP monolayer on HOPG acquired using excitation wavelengths of 514.5 nm and 441.6 nm exhibit similar band positions and relative intensities as those acquired under similar conditions of a film of neat NiOEP deposited on a glass slide, and with literature values acquired of solution samples using 514.5 nm and 406.7 nm excitation wavelengths, respectively. There are two bands in the spectra of the neat NiOEP film at 1578 cm⁻¹ and 1605 cm⁻¹ (indicated by the asterisks) that overlap with the large HOPG band in the spectrum of the monolayer. The band at 1578 cm⁻¹ is completely obscured by the HOPG peak, while the band at 1605 cm⁻¹ is visible on the right edge of the HOPG peak. The rest of the bands in the monolayer spectrum have counterparts in the spectrum of the pure NiOEP film.

The Raman shifts of all of the bands in the spectra of the monolayer and film acquired using both 514.5 nm and 441.6 nm excitation agree with literature reports for solution spectra, but intensities of some of the bands in the spectra of the thin films are different from those in the spectrum of the monolayer. Spectra were normalized for intensity using the band at 1316 cm⁻¹, which was chosen because it seems to have a similar intensity when excited at 514.5 nm and 406.7 nm, based on inspection and reported spectra, and provides good overlap of the baselines of the spectra being compared upon normalization. For the spectra acquired using 514.5 nm excitation, the peaks at 1655 cm⁻¹, 1520 cm⁻¹, 1383 cm⁻¹, 1225 cm⁻¹, 1025 cm⁻¹, 751 cm⁻¹, and 674 cm⁻¹ have greater relative intensity in the spectrum of NiOEP film, and the band at 1407 cm⁻¹ has greater relative intensity in the spectrum of the monolayer. For the spectra acquired using 441.6 nm excitation, the intensities of the peaks at 1383 cm⁻¹, 1157 cm⁻¹, 1025 cm⁻¹, 804 cm⁻¹, 751 cm⁻¹, and 674 cm⁻¹ are greater in the spectrum of the monolayer than in the spectrum of the NiOEP film. In the spectrum of the NiOEP film, only the peak at 1660 cm⁻¹ is taller than in the spectrum of the monolayer. The rest of the peaks are proportionally similar in both spectra. The cause of the intensity variation from the monolayer to the film is unclear, and more work would have to be done to determine if this is an important phenomenon or is within the realm of experimental error.

There are a number of bands in the spectra of MOEP compounds between 600 cm⁻¹ and 1700 cm⁻¹ that are known to be sensitive to variation of the central metal atom. These bands, which are assigned to C_{α}-Cₘ and C_{β}-C_{β} stretches by Spiro and co-workers (Spiro, T. G.; Czernuszewicz, R. S.; Li, X. Y., Coord. Chem. Rev. 1990, 100, 541-571), are typically located around, and sometimes obscured by, the HOPG band at 1580 cm⁻¹. The band at 1405 cm⁻¹ in each of the MOEP spectra is not present in the H₂OEP spectrum. The bands nearest the 1580 cm⁻¹ HOPG band are at different frequencies in all four MOEP spectra. The Raman bands of the monolayers generally agree well with those of the bulk porphyrin.

Confocal Raman Microscopy Measurements of Two-Component Mixed Monolayers of ZnOEP and H₂OEP on HOPG.

Mixtures of octaethylporphyrin form monolayers on HOPG whose structures resemble those of pure porphyrins. The mixed monolayers are compositionally disordered; ZnOEP molecules, which appear brighter in the images than H₂OEP molecules, are randomly distributed. The apparent heights for each type of molecule correlate well with those found in their one-component monolayers. Under these dosing conditions, there does not appear to be a preference for depositing one type of porphyrin over the other. The compositions of the two types of porphyrin in the monolayers determined by counting bright and less bright features, and by LDI-TOF MS analysis are similar to those of the dosing solution.

A typical confocal Raman microscope spectrum of a two-component mixed monolayer composed of 50% H₂OEP and 50% ZnOEP acquired using 514.5 nm excitation has bands that correspond to those in Raman spectra of the one-component monolayers of H₂OEP and ZnOEP. A number of the bands are present in all three spectra (indicated by the vertical gray lines), but there are a few bands that are unique to either H₂OEP or ZnOEP that appear in the spectrum of the mixed monolayer. In Raman spectra of monolayers formed from dosing solutions with 80%:20% and 20%:80% compositions of H₂OEP and ZnOEP, respectively, the peaks that were unique to one porphyrin did not appear to scale with the relative percentage of the porphyrin in the mixture. Without being bound by theory, it may be due in part to fluorescence emission of ZnOEP (excited at 514.5 nm) slightly overlapping with the spectral region of the Raman scattered light.

To determine the generality of this method for identifying the components of mixed monolayers, the Raman spectrum of a mixed monolayer of NiOEP and H₂OEP was studied. There are two key advantages to using this mixture of OEPs as compared to the H₂OEP/ZnOEP mixture: first, neither NiOEP nor H₂OEP have fluorescence overlap with the Raman scattered light when excited at 514.5 nm, which allows for a better signal-to-noise ratio in spectra; and second, since the composition of the NiOEP:H₂OEP monolayer was difficult to determine using the heights of features in STM images, this study also serves as a test case for qualitatively determining the monolayer composition by the presence of Raman bands from each analyte. The Raman spectrum of a mixed monolayer of NiOEP and H₂OEP clearly shows features from the two analytes.

The Raman spectra of octaethylporphyrin monolayers on HOPG were acquired on a confocal Raman microscope because the instrument had sufficient sensitivity to analyze samples with very low concentration of molecules on the surface (˜10¹³ molecules/cm²). The spectra acquired from monolayers show bands that agree well with Raman spectra of the same molecules in a film deposited on glass and with literature reports. The band that arises from the HOPG substrate at 1580 cm⁻¹ obscures bands from the porphyrin monolayer between ˜1560 cm⁻¹ and ˜1600 cm⁻¹.

Spectra of one-component monolayers of NiOEP, CoOEP, ZnOEP, and H₂OEP on HOPG are sufficiently different to allow Raman spectroscopy to be used as a fingerprint method to identify the MOEP compounds present in monolayers composed of mixtures of MOEPs. The majority of the bands in the region between 600 cm¹ and 1700 cm⁻¹ for typical monolayers of MOEP (e.g., those located at ˜1380 cm⁻¹, ˜1315 cm⁻¹, ˜1257 cm⁻¹, ˜1220 cm⁻¹, ˜1136 cm⁻¹, ˜1022 cm⁻¹, and ˜673 cm⁻¹) are found at the same frequency for all four porphyrins studied, but, there are some bands that shift depending on the identity of the metal center. These metal-sensitive bands are typically located around the 1580 cm⁻¹ HOPG band. The two-component monolayers of H₂OEP:ZnOEP and H₂OEP:NiOEP excited at 514.5 nm showed peaks from both types of porphyrin. However, the intensities of the metal-sensitive bands did not vary appreciably with monolayer composition (80% H₂OEP and 20% ZnOEP, 50% H₂OEP and 50% ZnOEP, and 20% H₂OEP and 80% ZnOEP).

The data acquired by excitation with the 441.6 nm laser had the best signal-to-noise ratio out of all of the excitation wavelengths studied, and produced the least fluorescence from ZnOEP, which made it the ideal excitation wavelength for this study. Raman spectra of NiOEP monolayers could be acquired using the longest excitation wavelength available with the instrument (632.8 nm), however, the fluorescence of ZnOEP and H₂OEP made it impossible to acquire spectra, illustrating the importance of tunability of the excitation wavelength.

Synthesis and Characterization of Five-Coordinate Gallium Octaethylporphyrin Building Blocks and Monolayers on HOPG

With sufficient understanding of the forces that drive the 2D ordering of planar molecules on solid substrates, the rational design of 3D surface supported materials is possible through utilization of square-pyramidal molecules that assemble into 2D ordered structures on surfaces with the axial ligands pointing up. Five-coordinate metallo-octaethylporphyrins are useful building blocks for developing the foundation of 3D molecular frameworks on surfaces. It is known that four-coordinate porphyrin molecules form ordered 2D monolayers on solid substrates with the plane of the molecule laying parallel to the surface, as described elsewhere herein. Five-coordinate metallo-porphyrins and phthalocyanines are expected to adsorb with the axial ligand pointing up, which renders it accessible for further chemical modification. If this axial ligand is bidentate with one end free, it can act as a binding point for attaching subsequent layers of molecules.

The assembly of four-coordinate porphyrins and phthalocyanines has been studied by scanning tunneling microscopy (STM) on a variety of substrates at low and room temperatures, and in a variety of environments, including at the solid/vacuum interface, at the solid/liquid interface and at the solid/air interface. There are only a handful of examples of five-coordinate metalloporphyrins or metallophthalocyanine overlayers on surfaces reported. Examples of five-coordinate porphyrin and phthalocyanine monolayers where the axial ligand is covalently bound to the metal center are imaged with STM under a variety of conditions, but the ligands are limited oxo-compounds such as V(O)OEP and Ti(O)OEP. Examples of five-coordinate porphyrin and phthalocyanine monolayers with more complex ligands tend to have ligands that are datively bound to the metal center; however, these monolayers are typically imaged at the solid-liquid interface at room temperature or the solid-vacuum interface at low temperature. There is one report of a five-coordinate Zn(L)OEP monolayer on highly oriented pyrolytic graphite (HOPG) imaged in air at room temperature with a datively bound ligand; however, there is some ambiguity to the report because only STM images were used to characterize the overlayer, the ligand-porphyrin complex does not look appreciably different from the porphyrin alone, and the presence of the ligand is not confirmed by any complementary analytical method.

The family of five-coordinate porphyrin monolayers on HOPG is enlarged by the characterization of five-coordinate gallium octaethylporphyrin (Ga(OEP)L) assemblies. Monolayers were dosed onto HOPG and imaged with STM in air at room temperature. Laser desorption/ionization time-of-flight mass spectrometry (LDI-TOF MS) and confocal Raman microscopy are used to independently probe the surface composition.

STM imaging of five-coordinate gallium octaethylporphyrin monolayers on HOPG.

Gallium Octaethylporphyrin Chloride.

Gallium octaethylporphyrin chloride (Ga(OEP)Cl) forms ordered monolayers on HOPG with the plane of the porphyrin oriented parallel to the substrate, and the chlorine atom pointed away from the surface. STM images acquired from a sample prepared by dosing Ga(OEP)Cl from a benzene solution onto HOPG show long-range ordering into close packed domains that range in size from tens to hundreds of nanometers across. Dosing experiments yielded quality images at a better than 60% success rate. Here we present images of Ga(OEP)Cl scanned using biases of −500 mV and +300 mV because these produced images with the best resolution for the purpose of describing the 2D structure and that illustrate the bias dependence of the heights of features in the cross sectional profiles of the overlayer.

An STM image of a monolayer of Ga(OEP)Cl on HOPG (75 nm×75 nm) shows part of a large domain. Individual porphyrin molecules appear as bright features, which are attributed to the heterocyclic portion of each molecule and to the Ga(Cl) centers, surrounded by a darker region, attributed to the region of the β-ethyl substituents at the periphery of the heterocycle. There are a number of defects in the monolayer, the most common of which are dark spots attributed to vacancies. These are randomly distributed in the monolayer. A cross sectional profile shows vacancies with apparent heights that are shorter than the surrounding porphyrins by ˜0.8 Å and ˜1 Å.

In STM images recorded at a smaller scan size (50 nm×50 nm), Ga(OEP)Cl monolayers appear to have a similar packing geometry to monolayers of planar MOEP compounds (M=H₂, Ni, Zn, Co). This is confirmed by the Ga(OEP)Cl lattice parameters, which are within experimental error of those found for MOEP. The image of NiOEP, which is representative of these compounds. The heights of features in Ga(OEP)Cl monolayers imaged at a negative bias are taller than in NiOEP monolayers. The profiles of the two compounds have a different shape at negative biases: the NiOEP molecules have a low spot at the center and the Ga(OEP)Cl molecules have a sharp spike at the center. Vacancies in the monolayer are highlighted by the circles.

The images of Ga(OEP)Cl recorded using positive biases show molecules with peaks at the center and feature heights that range from ˜5-8 Å, which is considerably taller than those for NiOEP and ZnOEP and compared to the 2-2.5 Å heights of Ga(OEP)Cl molecules recorded using a negative bias. The ˜5-8 Å apparent molecular heights are much taller than the ˜2.24 Å Ga—Cl bond length in the reported crystal structure. At both sample biases (−500 mV and +300 mV) the shapes of the features are similar, being wide at the base and narrowing sharply at the top. The high point is the expected location of the chloride ligand.

Gallium Octaethylporphyrin Phenylacetylide.

Gallium octaethylporphyrin phenylacetylide (Ga(OEP)CCPh) forms ordered monolayers on HOPG with the plane of the porphyrin oriented parallel to the substrate and the phenylacetylide ligand pointing up. Individual molecules aggregate into closely packed domains that range in size from tens to hundreds of nanometers across. Dosing of Ga(OEP)CCPh onto HOPG yielded monolayers of sufficient quality to acquire STM images in approximately 20% of experiments. The Ga(OEP)CCPh images presented here were scanned at the 1-phenyloctane/HOPG interface because these conditions produced images with the best quality images for the purpose of a discussion of the 2D and 3D structure of the overlayer. Images of Ga(OEP)CCPh were also acquired at the air/HOPG interface, but the images were of poorer quality than those acquired from samples in liquid. Scanning of monolayers of Ga(OEP)CCPh at biases between +200 mV and +800 mV produced images that were very noisy and did not have any noticeable surface patterning; thus images were not recorded using positive biases.

The STM images of a Ga(OEP)CCPh monolayer on HOPG show long range ordering of the molecules similar to Ga(OEP)Cl. The 2D packing in STM images of a monolayer of Ga(OEP)CCPh is similar to that of both Ga(OEP)Cl and MOEP monolayers on HOPG. There are defects in the monolayer where one or more porphyrin molecules are absent from the lattice. The cross sectional profile shows the regularity of the packing, and the apparent heights of the features (˜2 Å) are comparable to those of Ga(OEP)Cl imaged at a similar bias. This is somewhat surprising because the presence of the phenylacetylide ligand was expected to add approximately 6.3 Å to the apparent height of Ga(OEP)Cl.

Gallium Octaethylporphyrin Pyridylacetylide.

Gallium octaethylporphyrin pyridylacetylide (Ga(OEP)CCpy) forms ordered monolayers on HOPG with the plane of the porphyrin oriented parallel to the substrate and the pyridylacetylide ligand pointing up, similar to Ga(OEP)CCPh. STM images acquired from a sample prepared by dosing Ga(OEP)CCpy from a benzene solution onto HOPG show 2D ordering where individual molecules aggregate into domains. Images were acquired in approximately 10% of dosing experiments. Images of Ga(OEP)CCpy scanned at the 1-phenyloctane/HOPG interface are presented because these conditions produced images with the best resolution for the purpose of a discussion of the 2D structure of the overlayer. Images were also acquired at the air/HOPG interface that showed monolayers with similar packing geometry and height measurements, but the quality of the images was lower than those imaged in liquid.

An image of a Ga(OEP)CCpy one-component monolayer on HOPG (25 nm×25 nm) shows a closely packed domain with individual molecules appearing as bright spots with darker space in between. Images of Ga(OEP)CCpy were difficult to acquire. The lattice parameters found for monolayers of Ga(OEP)CCpy are similar to those of Ga(OEP)CCPh, Ga(OEP)Cl, and NiOEP monolayers on HOPG. The features in the STM image have apparent heights that are much shorter than those seen in monolayers of Ga(OEP)Cl and Ga(OEP)CCPh.

LDI-TOF MS measurements of one-component monolayers of five-coordinate gallium octaethylporphyrins on HOPG.

A typical LDI-TOF mass spectrum of a one-component Ga(OEP)Cl monolayer desorbed from HOPG at low laser power is dominated by a molecular ion peak at m/z=636. Ionization occurs primarily by loss of an electron to give Ga(OEP)Cl.⁺ radical cation similar to that seen for other MOEPs. The isotopic distribution of the molecular ion agrees with prediction. A second ionization pathway is also present where fragmentation of Ga(OEP)Cl occurs by loss of the Cl⁻ ligand to generate Ga(OEP)⁺ (m/z=601). The Ga(OEP)⁺ fragment is a common feature in electrospray ionization (ESI) mass spectra of five-coordinate gallium octaethylporphyrins with axial ligands, and it is observed in LDI-TOF MS experiments for each of the Ga(OEP)L molecules studied. Fragment ions corresponding to the elimination of a terminal methyl group from one of the β-ethyl substituents are also present.

At high laser powers, the Ga(OEP)Cl.⁺ parent ion all but disappears, and the most intense ion in the spectrum is the Ga(OEP)⁺ fragment. At very high laser powers, up to nine fragment peaks are observed, which correspond to the loss of the Cl⁻ axial ligand and methyl groups from up to eight of the β-ethyl substituents of the octaethylporphyrin. The intensities of all of the fragment ions increase with increasing laser power. There is no evidence of doubly charged Ga(OEP)Cl⁺⁺ molecular ions or fragments.

A comparison of typical LDI-TOF MS spectra of one-component monolayers of five-coordinate Ga(OEP)L, shows that the different kinds Ga(OEP)L monolayers can be identified using both their m/z ratio and their respective isotope distribution. The Ga(OEP)CCPh and Ga(OEP)CCpy have similar isotopic distributions of the molecular ions, which are distinctly different from that of Ga(OEP)Cl. Because GaOEP⁺ is a known ion occurring in LDI-TOF mass spectra of Ga(OEP)L molecules, it was used as an internal standard to mass calibrate data. This is critical to the process of distinguishing between these monolayers because Ga(OEP)CCPh (m/z=702) and Ga(OEP)CCpy (m/z=703) are only slightly different in their mass.

Confocal Raman Microscopy Measurements of Monolayers of Five-Coordinate Gallium Octaethylporphyrins on HOPG.

Raman spectra of porphyrin monolayers and thin films were recorded at the Center for Nanoscale Materials at Argonne National Laboratory using a confocal Raman microscope with several choices for excitation wavelength (441.6 nm, 514.5 nm, and 632.8 nm). In contrast to the four-coordinate porphyrin monolayers and thin films, however, only the 441.6 nm excitation wavelength could be used for data acquisition of the three five-coordinate Ga(OEP)L samples.

The fluorescence properties of Ga(OEP)Cl, Ga(OEP)CCPh, and Ga(OEP)CCpy account for the limitation of excitation wavelength to 441.6 nm. For both the 514.5 nm and 632.8 nm excitation wavelengths the Raman window overlaps with the fluorescence of all three molecules. When attempts were made to use either of these wavelengths for excitation, the detector became saturated, and no useful structural information about the analytes could be acquired. The 441.6 nm excitation wavelength, however, lies at the red tail of the Soret bands of all of the Ga(OEP)L molecules, and the Raman window is free of overlap with fluorescence, which allowed for collection of Raman spectra of the monolayers and thin films.

Similar to the Raman spectra of four-coordinate porphyrins, the Raman spectrum of a Ga(OEP)Cl monolayer shows that the bands between 600 cm⁻¹ and 1700 cm⁻¹ agree well with those of the pure Ga(OEP)Cl thin film on a glass slide as indicated by the gray dashed lines. A very large band at 1580 cm⁻¹, attributed to HOPG overlaps with a band in the spectrum of the pure sample at 1583 cm⁻¹. The Raman spectra of Ga(OEP)Cl has not been previously reported, so detailed assignments are not available. However, a comparison to the frequencies of NiOEP shows that there are many corresponding features. Metal dependence of the bands nearest the 1580 cm⁻¹ HOPG band are at different frequencies in the Raman spectra of monolayers of Ga(OEP)Cl, NiOEP, CoOEP, ZnOEP, and H₂OEP on HOPG excited at 441.6 nm.

The Raman spectra of Ga(OEP)Cl, Ga(OEP)CCPh, and Ga(OEP)CCpy are all similar, and there is no clear indication of a difference in their spectra that might be due to the presence of different ligands. For example, the CC stretch of the acetylide ligand, which would show up near 2200 cm⁻¹, is not present in the spectra of either Ga(OEP)CCPh or Ga(OEP)CCpy. This may be due to the vibrational modes of the axial ligand being decoupled from the vibrational modes of the porphyrin ring, similar to that reported by others for iron porphyrins with a variety of ligands, which means that the modes associated with the ligand would not benefit from the same resonance enhancement as the porphyrin portion of the molecule.

To extend 2D ordered monolayers up from the surface to form 3D surface-confined supramolecular frameworks, HOPG was dosed with five-coordinate metalloporphyrins: Ga(OEP)Cl, Ga(OEP)CCPh, and Ga(OEP)CCpy. Based on the lattice parameters measured from the STM images being similar to those of four-coordinate metalloporphyrins monolayers on HOPG, it can be concluded that the five-coordinate porphyrins lay down with the porphyrin heterocycle parallel to the substrate and the ligand pointing up.

Cross sectional profiles from STM images of five-coordinate Ga(OEP)L (L=Cl, CCPh) monolayers show features that are broad at the bottom and narrow sharply at the top for all biases where images were acquired. This is in contrast to the four-coordinate porphyrins, where cross sectional profiles of NiOEP and ZnOEP monolayers on HOPG imaged at negative bias show features that are broad at the base and narrow toward the top with a dip at the center. Ga(OEP)Cl and Ga(OEP)CCPh monolayers have similar height profiles in images captured using negative bias, which can be problematic for distinguishing between the molecules in mixed monolayers.

STM images of Ga(OEP)Cl monolayers tended to have the least noise and fewest defects; images of Ga(OEP)CCPh tended to have some noise and more point defects; and images of Ga(OEP)CCpy had the most noise and largest number of defects. This may be partially due to the structures of the Ga(OEP)CCpy and Ga(OEP)CCPh having ligands with nutational freedom. The movement of the ligands could cause images to be noisy. Ga(OEP)CCpy also has an accessible lone pair of electrons that may interact with the STM tip, the HOPG substrate, or other molecules on the surface, possibly adversely affecting the ordering or imaging of the monolayer.

Similar to the findings for four-coordinate MOEP monolayers, LDI-TOF MS is useful tool for the compositional characterization of five-coordinate Ga(OEP)L monolayers. Molecular ions formed by loss of an electron and fragment ions formed by cleavage of methyl groups from the β-ethyl substituents at the periphery of the heterocycle are present in LDI-TOF mass spectra of Ga(OEP)Cl, Ga(OEP)CCPh, and Ga(OEP)CCpy. A new type of fragmentation/ionization is also present for five-coordinate porphyrins where the molecule loses the axial ligand as a net anion, to form the Ga(OEP)⁺ ion. This type of fragmentation is useful because it shows that the gallium-ligand bond is readily cleavable, which means that tandem mass spectrometry (MS/MS) may be useful to analyze monolayers composed of molecules with complex ligands; and because Ga(OEP)⁺ is a known fragment, it can be used as an internal standard for in situ mass-calibration spectra. For example, LDI-TOF MS can distinguish between Ga(OEP)CCPh (m/z=702) and Ga(OEP)CCpy (m/z=703) monolayers when using the Ga(OEP)⁺ (m/z=601) as an internal standard.

In confocal Raman microscopy experiments of Ga(OEP)L monolayers it is important to select a wavelength that is well away from the fluorescence emission of the analyte in order to avoid saturating the detector. Using the 441.6 nm excitation wavelength to enabled the acquisition of Raman spectra of a monolayer of Ga(OEP)Cl, which agrees well with that of thin film of the pure compound deposited on a glass slide, with the exception of two peaks.

Because the spectra used here do not show intense peaks due to Ga—X and internal CCR modes, Raman spectroscopy may be of limited use in distinguishing between Ga(OEP)X components in mixed monolayers. This may be because they do not benefit from the same magnitude enhancement as the heterocyclic portion of the molecule.

### Experimentals

### Synthesis

General Procedures.

All synthesis experiments were performed under nitrogen atmosphere using standard Schlenk and glovebox techniques, except where noted. HPLC-grade solvents, stored under nitrogen in stainless-steel cylinders, were purified by passing them under nitrogen pressure through an anaerobic, stainless-steel system consisting of either two 4.5 in.×24 in. (1 gal) columns of activated A2 alumina (Et₂O, CH₂Cl₂, and THF) or one column of activated A2 alumina and one column of activated BASF R3-11 catalyst (toluene, pentane). NMR solvents (C₆D₆ and THF-d₈) were purified by stirring over NaK (1:2) alloy for 24 h, from which they were transferred under vacuum. All other reagents were obtained from commercial sources and used as received.

¹H- and ¹³C{¹H}-NMR spectra were recorded at room temperature using Bruker DMX 500 or DRX 400 MHz NMR spectrometers. Chemical shifts were measured relative to solvent resonances (¹H, ¹³C). Electronic-absorption spectra were recorded using an Agilent Technologies 8453 UV-Visible spectrophotometer of samples in quartz cuvettes (1 cm path length). Mass spectral data were obtained using a Voyager DE-PRO MALDI-TOF mass spectrometer on samples prepared on a standard 100 well stainless steel plate without a matrix.

Ga(OEP)Cl.

Ga(OEP)Cl was synthesized following a previously reported procedure (Coutsolelos, A.; Guilard, R.; Bayeul, D.; Lecomte, C., Polyhedron 1986, 5 (6), 1157-1164.). To a solution of gallium trichloride (GaCl₃) (128 mg, 0.727 mmol) and H₂OEP (200 mg, 0.374 mmol) in acetic acid (10 mL) was added a solution of NaOAc (200 mg) in acetic acid (17 mL). The solution was refluxed overnight, and the color changed from yellow-purple to pink-purple. The solution was cooled to 0° C. and filtered in air using filter paper and a Buchner funnel. Excess HOAc was removed in vacuo. The final product was isolated as a purple powder. Yield: 0.219 g, 92%. ¹H NMR (C₆D₆, 500.13 MHz): δ 10.42 (s, 4H, meso H), 7.21 (d, 2H, py), 4.85 (d, 2H, py), 3.99 (m, 16 H, βCH₂CH₃), 1.85 (t, 24H, βCH₂CH₃). LDI-TOF MS: m/z=531. UV-Vis (λₘₐₓ, nm): (CH₃OH) 333, 378, 398, 531, 569; (CH₂Cl₂) 333, 382, 402, 553, 571; (C₆H₅CH₃) 336, 385, 406, 536, 574.

HCCpy.

To a stirred, room temperature, green aqueous solution (50 mL) of commercial HCCpy.HCl (1.07 g, 7.73 mmol) in air was added NEt₃ (50 mL). The solution turned colorless and the HCCpy was extracted into Et₂O (3×100 mL). The organic phase was washed with H₂O (3×50 mL), dried with MgSO₄, and filtered, and solvents were removed in vacuo. The product was sublimed under vacuum at room temperature to give a white solid (0.555 g, 69.6% yield). ¹H NMR (CDCl₃, 400.13 MHz): δ 8.60 (d, 2H, py), 7.35 (d, 2H, py), 3.30 (s, 1H, CCH).

LiCCPh.

A solution of BuⁿLi(1.23 mL, 1.6 M hexanes solution, 1.96 mmol) was added to a −78° C. solution of HCCpy (0.225 g, 2.18 mmol) in pentane (50 mL). The reaction was stirred for 2 h whilst it was allowed to warm to room temperature. The resulting white precipitate was collected on a sintered glass funnel filter, washed with pentane, and dried in vacuo. The isolated white powder (0.237 g, 99% yield) was used without further purification.

LiCCpy.

A solution of BuⁿLi (3.5 mL, 1.6 M hexanes solution, 8.75 mmol) was added to a −78° C. solution of HCCPh (0.93 g, 9.1 mmol) in ether (50 mL). The reaction was stirred for 2 h whilst it was allowed to warm to room temperature. The resulting white precipitate was collected on a sintered glass funnel filter, washed with pentane, and dried in vacuo. The isolated white powder (0.883 g, 89% yield) was used without further purification

Ga(OEP)CCPh.

A solution of LiCCPh (0.030 g, 0.275 mmol) in THF (2 mL) was added to a stirred solution of Ga(OEP)Cl (0.050 g, 0.078 mmol) in toluene (25 mL) at room temperature. The color changed immediately from dark pink to dark purple. The solution was allowed to stir for 45 min, and then the solvent was removed in vacuo. The product was taken up in benzene, filtered through celite, and the solvent was removed in vacuo. The product was taken up in a minimal amount of benzene and purified by chromatography on a Fisher, 60-325 mesh basic alumina column (benzene) in air. The final product was isolated as a purple powder (0.053 g, 0.075 mmol, 96% yield). ¹H NMR (C₆D₆, 500.13 MHz): δ 10.40 (s, 4H, meso H), 5.99 (t, 1H,p-Ph), 5.82 (t, 2H, m-Ph), 5.34 (d, 2H, o-Ph), 3.98 (m, 16H, βCH₂CH₃), 1.84 (t, 24H, βCH₂CH₃). ¹³C{¹H} NMR (CDCl₃, 125.76 MHz): δ 146.23 (α), 142.39 (β), 130.65 (s, Ga═C≡C), 128.49 (s, o-Ph), 126.40 (s, m-Ph), 125.97 (s, p-Ph), 123.36 (s, ipso-Ph), 96.94 (s, C, meso), 20.11 (βCH₂CH₃), 18.71 (βCH₂CH₃). LDI-TOF MS: m/z=702.48; predicted, 702.32. UV-Vis (λₘₐₓ, nm): (C₆D₆) 340, 392, 413, 542, 580.

Ga(OEP)CCpy.

Ga(OEP)CCpy was synthesized by analogy to Ga(OEP)CCPh. A solution of LiCCpy (0.030 g, 0.275 mmol) in THF (2 mL) was added to a stirred solution of Ga(OEP)Cl (0.050 g, 0.078 mmol) in toluene (25 mL) at room temperature. The color changed immediately from dark pink to dark purple. The solution was allowed to stir for 45 min, and then the solvent was removed in vacuo. The product was extracted into toluene (25 mL) and filtered through celite. The volatile components were removed from the filtrate under vacuum. The final product was isolated as a purple powder. Yield: 0.052 g, 95%. ¹H NMR (C₆D₆, 500.13 MHz): δ 10.42 (s, 4H, meso H), 7.21 (d, 2H, py), 4.85 (d, 2H, py), 3.99 (m, 16H, βCH₂CH₃), 1.85 (t, 24H, βCH₂CH₃). ¹³C{¹H} NMR (C₆D₆, 125.76 MHz): δ 148.29 (s, o-C₅H₄N), 146.84 (α),142.69 (β), 130.79 (s, m-C₅H₄N), 128.25 (s, Ga—C≡C), 124.64 (s, ipso-C₅H₄N), 97.51 (s, C, meso), 20.24 (βCH₂CH₃), 18.68 (βCH₂CH₃). LDI-TOF MS: m/z=703.36; predicted, 703.31. UV-Vis (λₘₐₓ, nm): (C₆D₆) 413, 542, 578.

STM Sample Preparation.

Highly oriented pyrolytic graphite (SPI-2 grade) wafers measuring 10 mm×10 mm×1 mm, 7 mm×7 mm×1 mm, 10 mm×10 mm×2 mm were purchased from SPI Supplies and mounted to 12 mm diameter metal specimen discs with PELCO® Colloidal Silver Paste (Ted Pella, Inc.). A clean HOPG substrate was prepared immediately prior to dosing by cleaving the surface with adhesive tape. Benzene (CHROMOSOLV® Plus for HPLC, ≧99.9%) and metalloporphyrins (H₂OEP, 97%; NiOEP, 97%; and ZnOEP, 98%) were purchased from Sigma-Aldrich and used without further purification. Dosing solutions were prepared by dissolving porphyrin in benzene to a concentration of 2.5×10⁻⁴ M in 100 mL volumetric flasks. Two-component and three-component mixed porphyrin dosing solutions were prepared by combining stock solutions of the individual porphyrins in the desired ratios, for a total porphyrin concentration of 2.5×10⁻⁴ M. Two types of deposition were employed: immersion dosing and drop dosing. For MOEP monolayers prepared by immersion dosing, a freshly cleaved HOPG substrate was immersed in the porphyrin solution (˜1 mL) in a test tube at room temperature for 30-45 min. Samples were then removed from the dosing solution, rinsed with 2-3 drops of benzene applied with a Pasteur pipet, and placed under vacuum at room temperature for 2-3 hrs to remove residual solvent. This method is similar to that described in the literature for NiOEP on HOPG (Ogunrinde, A.; Hipps, K. W.; Scudiero, L., Langmuir 2006, 22 (13), 5697-5701 and Scudiero, L.; Hipps, K. W., Journal of Physical Chemistry C 2007, 111 (47), 17516-17520.). A second method of rinsing was used, where the dosed substrate was gently immersed in benzene immediately after it was removed from the dosing solution. This method for rinsing did not produce noticeably different monolayers. MOEP monolayers formed by drop dosing were prepared by applying a 0.6 μL drop of the dosing solution via micropipette to the center of the freshly cleaved HOPG substrate. The solvent was allowed to evaporate in air, and then the sample was rinsed and placed under vacuum at room temperature for 2-3 hrs. This method is similar to that described in the literature for V(O)OEP and CoOEP on HOPG (Miyake, Y.; Tanaka, H.; Ogawa, T., Colloids Surf, A 2008, 313, 230-233).

STM Measurements.

STM images were acquired using a Digital Instruments Nanoscope Ma standalone STM or Multimode STM. The platinum-iridium STM tips were formed from 0.25 mm Pt_{0.8}Ir_{0.2} (Goodfellow) wire either by mechanically cutting with clean titanium scissors or electrochemical etching in 8.0 M NaOH solution. Prior to use, tips were rinsed with deionized water to remove residual contaminants. All images were acquired in constant current mode. Samples were typically scanned at a variety of biases in order to both see the effect of bias on the images and to acquire the clearest possible images for analysis. After acquisition, images were processed using the WSxM 5.0 program (Horcas, I.; Fernandez, R.; Gomez-Rodriguez, J. M.; Colchero, J.; Gomez-Herrero, J.; Baro, A. M., Rev. Sci. Instrum. 2007, 78 (1), 013705). Images were flattened using the flatten tool and contrast was adjusted to sharpen images. In some images, a Gaussian smooth (lowpass filter) was applied to reduce noise.

Lattice Parameter Measurements.

Lattice parameters are extracted from images using the WSxM 5.0 program. A cross sectional profile (line scan) is drawn in one direction of the lattice formed by the porphyrin overlayer corresponding to unit cell direction a, b, or c. The line is drawn by first selecting the cross sectional profile tool (left-clicking on the button in the toolbar). Left-clicking in the image starts the line; then the line is formed by dragging the mouse a short distance and ended by right-clicking. The line can then be moved by clicking and dragging. The distance between troughs in the line scan, is then measured using the crosshair tool, where the distance between crosses is found in the blue oval at the bottom of the page. Lattice distances are defined as the spacing from porphyrin center to porphyrin center, but the same information can be measured more as the spacings between troughs, which are better defined in the images. The better definition of troughs compared to porphyrin centers is due to the fact that the centers of neighboring porphyrins do not always have the same shape, whereas the troughs between the porphyrins where tunneling occurs through the alkyl groups, consistently have a more symmetrical shape. For each image, 150-200 trough-to-trough measurements are recorded for each unit cell direction (a, b, and c). Lattice angles are also recorded using the cross sectional profile tool. The line is begun in the same way as for distance measurements, but after left-clicking on the image, a short line is drawn and the end of that segment is terminated by left-clicking; then the next segment is drawn and the line scan is completed by right-clicking. This gives two line segments connected at a vertex, which can be manipulated by left-clicking at the end of any line or the vertex and dragging to the desired location. When holding and dragging at the end of a line, a measurement of the angle between the two lines can be seen in the red oval at the bottom of the image. The cross sectional profile tool is used in this way to record each angle (<ab, <ac, and <bc) for 40 data points, where the vertex of a point corresponds to the center of a porphyrin molecule in the image. The extracted data are then loaded into an Excel spreadsheet to calculate an average and standard deviation for each length and angle. Lattice parameters are defined as the two shorter lengths of the triangle (abc) and the angle between them.

Ga(OEP)(CCpy).

A solution of LiCCpy (0.030 g, 0.275 mmol) in THF (2 mL) was added to a stirred solution of Ga(OEP)Cl (0.050 g, 0.078 mmol) in toluene (25 mL) at room temperature. The color changed immediately from dark pink to dark purple. The solution was allowed to stir for 45 min, and then the solvent was removed in vacuo. The product was extracted into toluene (25 mL) and filtered through celite. The volatile components were removed from the filtrate under vacuum. The final product was isolated as a purple powder. Yield: 0.052 g, 95%. ¹H NMR (C₆D₆, 500.13 MHz): δ 10.42 (s, 4H, meso H), 7.21 (d, 2H, py), 4.85 (d, 2H, py), 3.99 (m, 16H, βCH₂CH₃), 1.85 (t, 24H, βCH₂CH₂). ¹³C{¹H} NMR (C₆D₆, 125.76 MHz): δ 148.29 (s, o-C₅H₄N), 146.84 (α),142.69 (β), 130.79 (s, m-C₅H₄N), 128.25 (s, Ga—C≡C), 124.64 (s, ipso-C₅H₄N), 97.51 (s, C, meso), 20.24 (βCH₂CH₃), 18.68 (βCH₂CH₃). LDI-TOF MS: m/z=703.36; predicted, 703.31. UV-Vis (λ_{max,} nm): (C₆D₆) 413, 542, 578.

Ga(OEP)(CCPPh₂).

A solution of LiCCPPh₂ (0.034 g, 0.157 mmol) in THF (1 mL) was added to a stirred solution of Ga(OEP)Cl (0.047 g, 0.074 mmol) in toluene (25 mL) at room temperature. The color changed immediately from dark pink to red-purple. The solution was allowed to stir for 1.5 h, and then the solvent was removed in vacuo. The product was extracted into toluene (25 mL) and filtered through celite. The volatile components were removed from the filtrate under vacuum. The isolated purple powder was subsequently washed with aliquots of cold ether to remove excess LiCCPPh₂ (the product is somewhat soluble in ether). Yield: 0.033 g, 47%. ¹H NMR (C₆D₆, 500.13 MHz): δ 10.39 (s, 4H, meso H), 6.73 (m, 2H, p-Ph), 6.63 (m, 4H, m-Ph), 5.90 (m, 4H, o-Ph), 3.98 (m, 16H, βCH₂CH₃), 1.84 (t, 24H, βCH₂CH₃). ³¹P{¹H} NMR (C₆D₆, 202.45 MHz): δ −35.45 (s). ¹³C{¹H} NMR (C₆D₆, 125.77 MHz): δ 146.74 (s, α), 142.64 (s, β), 138.08 (d, i-Ph), 131.71 (d, o-Ph), 127.54 (d, m-Ph), 127.41 (p-Ph), 97.34 (s, meso), 20.24 (s, βCH₂CH₃), 18.74 (s, βCH₂CH₃).

Synthesis.

The gallium porphyrin acetylide complexes Ga(TPP)CCPrⁿ, Ga(TPP)CCPh, and Ga(OEP)CCPh have been previously synthesized by Balch and Guilard (Balch, A. L.; Latosgrazynski, L.; Noll, B. C.; Phillips, S. L., Inorg. Chem. 1993, 32 (7), 1124-1129, Kadish, K. M.; Boisseliercocolios, B.; Coutsolelos, A.; Mitaine, P.; Guilard, R., Inorg. Chem. 1985, 24 (26), 4521-4528 and Kadish, K. M.; Cornillon, J. L.; Coutsolelos, A.; Guilard, R., Inorg. Chem. 1987, 26 (25), 4167-4173). The former reported a specific procedure, shown in Equation 1, while the latter reported a general procedure for related gallium porphyrin alkyl complexes shown in Equation 2. Our initial attempts to prepare Ga(OEP)CCR followed Eq. 2 because this compound was synthesized by this procedure and the general yield was higher than for Eq. 1. Using LiCCPh as the acetylide reagent, several attempts provided messy reactions. We explored synthesis via Eq. 1, which differed from Eq. 2 by having a larger excess of LiCCR and eliminating the reaction quenching step with water. Ultimately, higher yields could be obtained by increasing the reaction time to 45 min rather than the 5 min reported by Balch. These changes improved the isolated yield from the reported 33% to >90%. The Ga(OEP)CCPh was purified by chromatography (benzene on basic alumina) similar to that reported by Balch, et al., but the Ga(OEP)CCpy decomposed on the column. In order to purify Ga(OEP)CCpy, after the solvent was removed, the product was taken up in a minimal amount of toluene and filtered through celite.

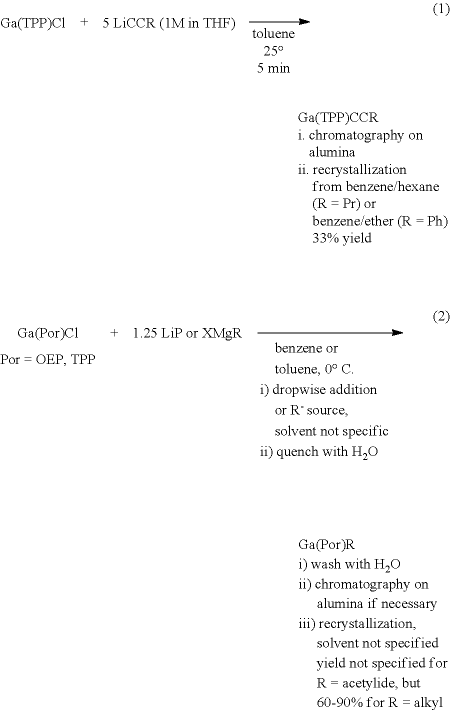

Depolarized confocal Raman microscopy spectra were acquired on a Renishaw InVia Confocal Raman Microscope at the Center for Nanoscale Materials (CNM) at Argonne National Laboratory (ANL). Spectra were measured using a 50× microscope objective to collect scattered light and to focus the laser excitation (5 mW). Excitation wavelengths of 514.5 nm (argon-ion laser), 441.6 nm (helium-cadmium laser), and 632.8 nm (helium-neon laser) were used. Monolayers of OEP on HOPG substrates and OEP films on glass slides (prepared using the same dosing solution as for the HOPG substrates) were placed on the sample stage, and the microscope was manually focused on the substrate by adjusting the height of the sample stage with respect to the microscope objective. Prior to use and at regular intervals over the course of 6-10 hours of measurements, the instrument was calibrated for intensity and wavelength with the quick calibration option in the WIRE 3.3 software package, using the 521 cm⁻¹ band of a silicon standard as the reference. Spectral data were acquired in both the static mode, for small spectral window acquisitions (˜750 cm⁻¹), and in the extended mode, for larger spectral window acquisitions (>750 cm⁻¹). Scattered light was collected on a CCD detector with the cosmic-ray-removal setting turned on. The time allotted to collect scattered light during acquisition of an individual spectrum (typically 10 s to 30 s) was optimized prior to each experiment. For each experiment, five to fifteen spectra were accumulated to increase the signal-to-noise ratio of the data. Spectra were initially saved as .wxd files, and then the files were converted to .txt files using a batch file converter for processing in Excel.

From the foregoing, it will be observed that numerous variations and modifications may be effected without departing from the spirit and scope of the invention. It is to be understood that no limitation with respect to the specific embodiment illustrated herein is intended or should be inferred. It is, of course, intended to cover by the appended claims all such modifications as fall within the scope of the claims.

1. A compound of formula (I)
MOL-M-L (I)
wherein M is a 5 or 6-coordinate metal,
L is a bidentate ligand, and
MOL is a compound of formula (II) 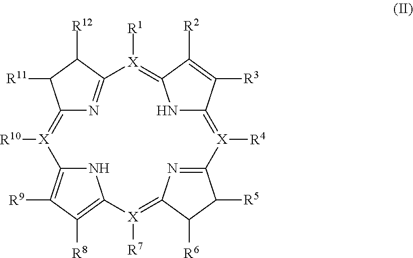
wherein X is C or N,
R¹, R², R³, R⁴, R⁵, R⁶, R⁷, R⁸, R⁹, R¹⁰, R¹¹ and R¹² are each independently selected from the group consisting of hydrogen, halogen, substituted or unsubstituted C₁₋₈ alkyl, substituted or unsubstituted C₂₋₈ alkenyl, substituted or unsubstituted C₂₋₈ alkynyl, —N₃, —OCN, —SCN, —NO₃, —OTeF₅, trazolate, tetrazolate, —CN, —OR¹³, —OOR¹³, —CCR¹³, —OC(O)R¹³, —CO₂R¹³, —C(O)R¹³, —C(O)NR¹³R¹⁴, —OC(O)NR¹³R¹⁴, —NR¹⁴C(O)R¹³, —NR¹⁵C(O)NR¹³R¹⁴, —NR¹³R¹⁴, —NR¹⁴CO₂R¹³, —SR¹³, —S(O)R¹³, —S(O)₂R¹³, —S(O)₂NR¹³R¹⁴, —NR¹⁴S(O)₂R¹³, substituted or unsubstituted C₆₋₁₀ aryl, substituted or unsubstituted 5- to 10-membered heteroaryl, substituted or unsubstituted 3- to 10-membered cyclyl, and substituted or unsubstituted 3- to 10-membered heterocyclyl, or one or more pairs of R²/R³, R⁵/R⁶, R⁸/R⁹ and R¹¹/R¹² are each independently, together with the atom(s) to which they are attached, form an substituted or unsubstituted 3- to 10-membered ring,
wherein each occurrence of R¹³, R¹⁴ and R¹⁵ is independently selected from the group consisting of hydrogen, C₁₋₈ alkyl, C₂₋₈ alkenyl, C₂₋₈ alkynyl, aryl, or heteroaryl; or R¹³ and R¹⁴, together with the atom(s) to which they are attached, form an substituted or unsubstituted 3- to 10-membered ring.

2. The compound of claim 1, wherein M bonds to MOL covalently.

3. The compound of claim 1, wherein M bonds to MOL datively.

4. The compound of claim 1, wherein M bonds to MOL both covalently and datively.

5. The compound of claim 1, wherein M bonds to L covalently.

6. The compound of claim 1, wherein M bonds to L non-covalently.

7. The compound of claim 1, wherein M is a 5-coordinate metal, M bonding to MOL with 4 coordinates and to L with 1 coordinate.

8. The compound of any of claim 1, wherein M is a 6-coordinate metal, M bonding to MOL with 4 coordinates and to L with 2 coordinates.

9. The compound of claim 1, wherein M is selected from the group consisting of Al, Bi, Co, Cr, Dy, Er, Eu, Fe, Ga, Gd, Ge, Hf, Hg, Ho, In, Ir, Ln, Lu, Mg, Mn, Mo, Nb, Nd, Os, P, Pc, Pd, Pr, Re, Rh, Ru, Sb, Sc, Si, Sm, Sn, Ta, Tb, Tc, Te, Ti, Tm, U, V, W, Y, Yb, Zn, and Zr.

10. The compound of claim 1, wherein M is Ga.

11. The compound of any of claim 1, wherein X is N.

12. The compound of claim 11, wherein R¹, R⁴, R⁷ and R¹⁰ are hydrogen.

13. The compound of claim 1, wherein X is C.

14. The compound of claim 13, wherein R¹, R⁴, R⁷ and R¹⁰ are each independently selected from the group consisting of
—(CH2)mCH3, 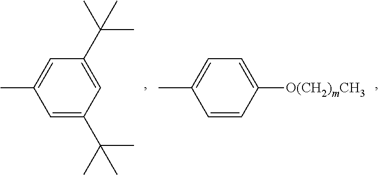
—COOH, a substituted or unsubstituted naphthalene diimide, —(CC—Ph)m-(CH2)mCH3, 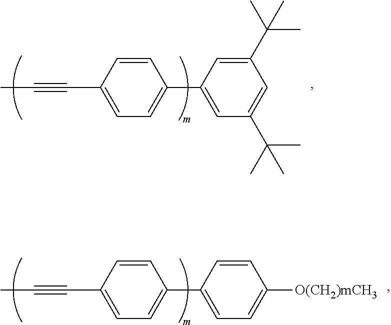
—(CC-Ph)ₘ—COOH, —(CC-Ph)ₘ-(a substituted or unsubstituted naphthalene diimide), wherein each occurrence of m is each independently from 0-30.

15. The compound of claim 1, wherein MOL is substituted or unsubstituted phthalocyanine.

16. The compound of claim 1, wherein MOL is substituted or unsubstituted tetrabenzophthalocyanine.

17. The compound of claim 1, wherein MOL is substituted or unsubstituted porphyrin.

18. The compound of claim 1, wherein MOL is substituted or unsubstituted tetrabenzoporphyrin.

19. The compound of claim 1, wherein L is selected from the group consisting of —CC-4-pyridyl, —CCPPh₂, —O(CH₂CH₂)ₙOCH₃, wherein n is from 0 to 15.

20. The compound of claim 1, wherein the bidentate ligand L comprises a functional group that is not bound to M.

21. The compound of claim 20, wherein the functional group that is not bound to M is selected from the group consisting of pyridine, amide, phosphine, NHC, acetylide, isocyanide, cyclopentadienyl, a half-sandwich pi ligand, alkoxide, thiolate, diimine, polypyridine, pyrazolyl borate, carboxylate, amine, ether, polyether, macrocyclic or an open O,N-containing ligand and combinations thereof.

22. The compound of claim 20, wherein the functional group that is not bound to M bonds, covalently or noncovalently, to a catalyst, a component of a catalytic system, a peptide, a protein, a virus, a small molecule, a drug, a carbohydrate, a bacterium, a nano particle, a chromophore, a luminophore, a magnetic molecule, a molecular magnet, a magnetic particle, a molecular electronic component, a fullerene, a nanostructured carbon cluster, a transition metal cluster and combinations thereof.

23. The compound of claim 1, which is Ga(β-octaethylporphyrin)(CC-4-pyridyl).

24. The compound of claim 1, which is Ga(β-octaethylporphyrin)(CCPPh₂).

25. A system, comprising:
a substrate,
a first compound of claim 1 immobilized on the substrate.

26. The system of claim 25, wherein the first compound forms a self-assembled monolayer on the substrate.

27. The system of claim 25, further comprising a second compound co-immobilized with the first compound on the substrate.

28. The system of claim 27, wherein the second compound is of formula (I) but is different from the first compound.

29. The system of claim 27, wherein the second compound has an oligo(ethylene glycol) terminal group.

30. The system of claim 27, wherein the second compound is H₂-MOL.

31. The system of claim 27, wherein the first compound forms at least one hydrogen bond with the second compound.

32. The system of claim 25, wherein the substrate is selected from the group consisting of Au, Ag, Cu, Ru, Si. T, highly oriented pyrolytic graphite, and combinations thereof.

33. The system of claim 25, wherein the substrate is Au.

34. The system of claim 25, wherein the substrate is highly oriented pyrolytic graphite.

35. The system of claim 25, wherein the first compound is immobilized on the substrate covalently.

36. The system of claim 25, wherein the first compound is immobilized on the substrate non-covalently.

37. The system of claim 25, wherein the bidentate ligand of the first compound is bound to M and to a moiety that is active as a component in a catalytic system, an information storage system, a sensor, an electron transfer system, a photon transfer system, or a structural unit that supports a layer of a nanomaterial disposed on the substrate or guides the orientation of a nanomaterial disposed on the substarte.

38. The system of claim 37, wherein the nanomaterial is grapheme, carbon nanotube, a nanostructure, or a polymer.

39. A method, comprising:
contacting a solution comprising a first compound of claim 1 with a substrate such that some of the first compound is immobilized to the substrate.

40. The method of claim 39, wherein the solution further comprises a second compound and wherein both some of the first compound and some of the second compound are immobilized to the substrate.

41. A method, comprising:
introducing a reagent to a substrate presenting the compound of claim 1.
